(19) United States
(12) Patent Application Publication
(10) Pub. No.: US 2025/0129362 A1
(43) Pub. Date: Apr. 24, 2025

(54) PREPARATION OF LIBRARIES OF PROTEIN VARIANTS EXPRESSED IN EUKARYOTIC CELLS

(71) Applicant: Iontas Limited, London (GB)

(72) Inventors: Kothai Nachiar Devi PARTHIBAN, Cambridge (GB); John MCCAFFERTY, Cambridge (GB)

(21) Appl. No.: 18/683,728

(22) PCT Filed: Aug. 24, 2022
(86) PCT No.: PCT/EP2022/073549

§ 371 Date: Feb. 14, 2024

(30) Foreign Application Priority Data
Aug. 25, 2021 (EP) 21193102.7

(51) Int. Cl. C12N 15/10

(52) U.S. Cl. CPC C12N 15/1082

(57) ABSTRACT

Described herein is a method for identifying a locus in a genome of a eukaryotic cell, said locus being a candidate for insertion of binder sequences. Described herein as well is a method of producing a library of eukaryotic cell clones containing DNA encoding a diverse repertoire of binders.

### FIELD

The current invention relates to the production of libraries of eukaryotic cell clones, specifically to libraries of eukaryotic cell clones containing DNA encoding a diverse repertoire of binders. Furthermore, the invention relates to methods identifying a locus in a genome of a eukaryotic cell.

### BACKGROUND

WO2015/166272 describes a method of producing a library of eukaryotic cell clones containing DNA encoding a diverse repertoire of binders. In this method, a site-specific nuclease is used to cleave a recognition sequence in cellular DNA, creating an integration site at which donor DNA encoding the binders can be integrated.

However, the method of WO2015/166272 relies on the choice of a suitable recognition sequence at specific loci in a genome of a eukaryotic cell to allow for the generation of a library characterized by a high diversity of binders and/or a uniform integration of binders and/or and a uniform transcription of binders. WO2015/166272 does not provide an extensive list of such recognition sequences or loci.

Thus, there is a continuing need in the art for a method for identifying a locus in a genome of a eukaryotic cell, said locus being a candidate for insertion of binder sequences, and for specific loci in a genome of a eukaryotic comprising suitable recognition sequences which may be used in a method for producing a library.

### SUMMARY

In an aspect, there is provided a method for identifying a locus in a genome of a eukaryotic cell, said locus being a candidate for insertion of binder sequences, said method comprising:
a. providing a landing pad sequence;
b. introducing the landing pad sequence into the eukaryotic cell;
c. randomly integrating the landing pad sequence into the genome of the eukaryotic cell via transposon-mediated integration;
d. selecting a clone having a landing pad sequence integrated into its genome.

A method according to this aspect may be called “a method for identifying a locus according to the invention” or “a method for identifying a locus” or the like in the context of this application.

In some embodiments, a method for identifying a locus according to the invention comprises the further steps of:
e. screening for single-copy integration;
f. identifying the locus.

In some embodiments, a method for identifying a locus according to the invention comprises the additional steps of:
g. integrating a donor DNA sequence comprising one or more transgenes encoding a binder at the landing pad sequence;
h. screening for integration of the donor DNA.

In some embodiments, a method for identifying a locus according to the invention is such that the landing pad sequence comprises a recognition sequence for a site-specific nuclease. Preferably, the nuclease recognition sequence is a meganuclease recognition sequence, a zinc finger nuclease recognition sequence, a TALE nuclease recognition sequence or a nucleic acid guided nuclease recognition sequence, more preferably a meganuclease recognition sequence, most preferably a I-SceI meganuclease recognition sequence.

In some embodiments, a method for identifying a locus according to the invention is such that step g of integrating the donor DNA into the cells comprises providing a site-specific nuclease within the cells, wherein the nuclease cleaves the recognition sequence comprised in the landing pad. In some embodiments, a method for identifying a locus according to the invention is such that step h of screening for integration of the donor DNA comprises screening for display of the one or more binders encoded by the donor DNA. In some embodiments, the donor DNA further comprises homology arms to increase integration efficiency. In some embodiments, the landing pad sequence and/or the donor DNA sequence comprise a selectable marker.

In an aspect, there is provided the use of the locus identified in a method for identifying a locus according to the invention for building a library of eukaryotic cell clones containing DNA encoding a diverse repertoire of binders. A use according to this aspect may be called “a use of a locus according to the invention” or the like in the context of this application.

In an aspect, there is provided a method for producing a library of eukaryotic cell clones containing DNA encoding a diverse repertoire of binders, comprising:
providing donor DNA molecules encoding the binders, and eukaryotic cells;
introducing the donor DNA into the cells and providing a site-specific nuclease within the cells, wherein the nuclease cleaves a recognition sequence in cellular DNA, wherein the recognition sequence is in an NLN gene, a TNIK gene, a PARP11 gene, a RAB40B gene, an ABI2 gene, an RNF19B gene, a PKIA gene, or an FTCD gene, to create an integration site at which the donor DNA becomes integrated into the cellular DNA, integration occurring through DNA repair mechanisms endogenous to the cells, thereby creating recombinant cells containing donor DNA integrated in the cellular DNA; and
culturing the recombinant cells to produce clones, thereby providing a library of eukaryotic cell clones containing donor DNA encoding the repertoire of binders.

A method according to this aspect may be called “a method for generating a library according to the invention” or “a method for generating a library” or “a method for producing a library” or the like in the context of this application. “A method according to the invention” refers to both a method for identifying a locus according to the invention and a method for generating a library according to the invention.

In some embodiments, a method for generating a library according to the invention is such that the recognition sequence is in an NLN gene, a TNIK gene or a RAB40B gene. Preferably the recognition sequence is in an NLN gene.

In some embodiments, a method for generating a library according to the invention is such that the recognition sequence is in an intron of the gene. In some embodiments, the recognition sequence is in an open chromatin region of the intron. In some embodiments, the recognition sequence is in an enhancer region of the intron.

For multimeric binders comprising at least a first and second subunit (i.e., separate polypeptide chains, such as antibody VH and VL domains presented within a Fab or IgG format), the multiple subunits may be encoded on the same molecule of donor DNA. However, it may be desirable to integrate the different subunits into separate loci, in which case the subunits can be provided on separate donor DNA molecules. These could be integrated within the same cycle of nuclease-directed integration or they may be integrated sequentially using nuclease-directed integration for one or both integration steps.

Methods of producing libraries of eukaryotic cell clones encoding multimeric binders may comprise:
providing eukaryotic cells containing DNA encoding the first subunit, and providing donor DNA molecules encoding the second binder subunit.
introducing the donor DNA into the cells and providing a site-specific nuclease within the cells, wherein the nuclease cleaves a recognition sequence in cellular DNA, wherein the recognition sequence is in an NLN gene, a TNIK gene, a PARP11 gene, a RAB40B gene, an ABI2 gene, an RNF19B gene, a PKIA gene, or an FTCD gene, to create an integration site at which the donor DNA becomes integrated into the cellular DNA, integration occurring through DNA repair mechanisms endogenous to the cells, thereby creating recombinant cells which contain donor DNA integrated in the cellular DNA. These recombinant cells will contain DNA encoding the first and second subunits of the multimeric binder, and may be cultured to express both subunits. Multimeric binders are obtained by expression and assembly of the separately encoded subunits.

In the above example, nuclease-directed integration is used to integrate DNA encoding a second subunit into cells already containing DNA encoding a first subunit. The first subunit could be previously introduced using the techniques of the present invention or any other suitable DNA integration method. An alternative approach is to use nuclease-directed integration in a first cycle of introducing donor DNA, to integrate a first subunit, followed by introducing the second subunit either by the same approach or any other suitable method. If the nuclease-directed approach is used in multiple cycles of integration, different site-specific nucleases may optionally be used to drive nuclease-directed donor DNA integration at different recognition sites. A method of producing libraries of eukaryotic cell clones encoding multimeric binders may comprise:
providing first donor DNA molecules encoding the first subunit, and providing eukaryotic cells
introducing the first donor DNA into the cells and providing a site-specific nuclease within the cells, wherein the nuclease cleaves a recognition sequence in cellular DNA, wherein the recognition sequence is in an NLN gene, a TNIK gene, a PARP11 gene, a RAB40B gene, an ABI2 gene, an RNF19B gene, a PKIA gene, or an FTCD gene, to create an integration site at which the donor DNA becomes integrated into the cellular DNA, integration occurring through DNA repair mechanisms endogenous to the cells, thereby creating a first set of recombinant cells containing first donor DNA integrated in the cellular DNA,
culturing the first set of recombinant cells to produce a first set of clones containing DNA encoding the first subunit,
introducing second donor DNA molecules encoding the second subunit into cells of the first set of clones, wherein the second donor DNA is integrated into cellular DNA of the first set of clones, thereby creating a second set of recombinant cells containing first and second donor DNA integrated into the cellular DNA, and
culturing the second set of recombinant cells to produce a second set of clones, these clones containing DNA encoding the first and second subunits of the multimeric binder,
thereby providing a library of eukaryotic cell clones containing donor DNA encoding the repertoire of multimeric binders.

“A method for generating a library according to the invention” and “a method according to the invention” as used herein also refer to the above methods of producing libraries of eukaryotic cell clones encoding multimeric binders.

Site-specific integration of donor DNA into cellular DNA creates recombinant cells, which can be cultured to produce clones. Individual recombinant cells into which the donor DNA has been integrated are thus replicated to generate clonal populations of cells—“clones”—each clone being derived from one original recombinant cell. Thus, the method generates a number of clones corresponding to the number of cells into which the donor DNA was successfully integrated. The collection of clones form a library encoding the repertoire of binders (or, at an intermediate stage where binder subunits are integrated in separate rounds, the clones may encode a set of binder subunits). Methods of the invention can thus provide a library of eukaryotic cell clones containing donor DNA encoding the repertoire of binders.

Accordingly, in an aspect, there is provided a library of eukaryotic cell clones containing DNA encoding a diverse repertoire of binders, wherein the library is obtained via use of a locus according to the invention and/or via a method for generating a library according to the invention. Such libraries according to this aspect may be called a “library of the invention” or the like in the context of this application.

Methods of the invention can generate libraries of clones containing donor DNA integrated at a fixed locus, or at multiple fixed loci, in the cellular DNA. By “fixed” it is meant that the locus is the same between cells.

Cells used for creation of the library may therefore contain a nuclease recognition sequence at a fixed locus, representing a universal landing site in the cellular DNA at which the donor DNA can integrate. The recognition sequence for the site-specific nuclease may be present at one or more than one position in the cellular DNA. Accordingly, in an aspect, there is provided an in vitro library of eukaryotic cell clones that express a diverse repertoire of at least 10\hat{ }3, 10\hat{ }4, 10\hat{ }5, 10\hat{ }6, 10\hat{ }7, 10\hat{ }8 or 10\hat{ }9 different binders, each cell containing recombinant DNA wherein donor DNA encoding a binder or subunit of a binder is integrated in a fixed locus in the cellular DNA, the locus being identified by a method according to the invention. Also provided is an in vitro library of eukaryotic cell clones according to the invention, wherein donor DNA encoding a binder or subunit of a binder is integrated in at least a first and/or a second fixed locus in the cellular DNA, wherein said fixed locus or loci are identified by a method according to the invention. A “library of the invention” or the like as used herein also refers to such an in vitro library of eukaryotic cell clones.

Libraries produced according to the present invention may be employed in a variety of ways. A library may be cultured to express the binders, thereby producing a diverse repertoire of binders. A library may be screened for a cell of a desired phenotype, wherein the phenotype results from expression of a binder by a cell. Accordingly, in an aspect, there is provided a method of screening for a cell of a desired phenotype, wherein the phenotype results from expression of a binder by the cell, the method comprising
providing a library via the method for producing a library of the invention, or providing a library via the use of a locus according to the invention, or providing a library according to the invention,
culturing the library cells to express the binders, and
detecting whether the desired phenotype is exhibited.

A method according to this aspect may be called “a method of screening for a cell of a desired phenotype according to the invention” or the like. “A method according to the invention” as used herein also refers to the above method of screening for a cell of a desired phenotype.

Phenotype screening is possible in which library cells are cultured to express the binders, followed by detecting whether the desired phenotype is exhibited in clones of the library. Cellular read-outs can be based on alteration in cell behaviour such as altered expression of endogenous or exogenous reporter genes, differentiation status, proliferation, survival, cell size, metabolism or altered interactions with other cells. When the desired phenotype is detected, cells of a clone that exhibits the desired phenotype may then be recovered. Optionally, DNA encoding the binder is then isolated from the recovered clone, providing DNA encoding a binder which produces the desired phenotype when expressed in the cell.

A key purpose for which eukaryotic cell libraries have been used is in methods of screening for binders that recognise a target of interest. Accordingly, in an aspect, there is provided a method for screening to identify a binder to a target of interest, said method comprising:
providing a library via the method for producing a library of the invention, or providing a library via the use of a locus according to the invention, or providing a library according to the invention,
culturing cells of the library to express the binders,
exposing the binders to the target, allowing recognition of the target by one or more cognate binders, if present, and
detecting whether the target is recognised by a cognate binder.

A method according to this aspect may be called “a method for screening to identify a binder to a target of interest according to the invention” or “a method for screening to identify a binder” or the like. “A method according to the invention” as used herein also refers to the above method for screening to identify a binder 20 to a target of interest.

In such methods a library is cultured to express the binders, and the binders are exposed to the target to allow recognition of the target by one or more cognate binders, if present, and detecting whether the target is recognised by a cognate binder. In such methods, binders may be displayed on the cell surface and those clones of the library that display binders with desired properties can be isolated. Thus cells incorporating genes encoding binders with desired functional or binding characteristics could be identified within the library. The genes can be recovered and used for production of the binder or used for further engineering to create derivative libraries of binders to yield binders with improved properties.

In an aspect, the invention also encompasses a binder that has been identified from a library of the invention, for example a binder that was identified using a method for screening to identify a binder to a target of interest according to the invention. Preferred binders are described elsewhere herein.

Various features of the invention are further described below. It is noted that headings used throughout this specification are to assist navigation only and should not be interpreted as definitive, and that features described in different sections may be relevant for all aspects of the invention and may be combined as appropriate.

### DETAILED DESCRIPTION

As shown in the experimental part, aspects of this invention such as the new loci of the invention are associated with advantages such as increased integration efficiencies and stable antibody expression.

### Eukaryotic Cells

Preferred eukaryotic cells and eukaryotic cell clones for aspects of this invention including the methods, uses and libraries of the invention are defined below. It is understood that all preferences relating to eukaryotic cells may also be applied to eukaryotic cell clones.

Eukaryotic cells are preferably higher eukaryotic cells, defined here as cells with a genome greater than that of Saccharomyces cerevisiae which has a genome size of 12×10⁶ base pairs (bp). The higher eukaryotic cells may for example have a genome size of greater than 2×10⁷ base pairs. This includes, for example, mammalian, avian, insect or plant cells. A eukaryotic cell is not limited to a mammalian cell. Preferably eukaryotic cells are mammalian cells, e.g., mouse or human. More preferably, eukaryotic cells are human cells. The eukaryotic cells may be primary cells or may be cell lines. Chinese hamster ovary (CHO) cells are commonly used for antibody and protein expression but any alternative stable cell line such as HEK293 cells may be used in the invention. Methods are available for efficient introduction of foreign DNA into primary cells allowing these to be used (e.g., by electroporation where efficiencies and viabilities up to 95% have been achieved http://www.maxcyte.com/technology/primary-cells-stem-cells.php).

A particular benefit of nuclease-directed integration comprised in a method for identifying a locus or in a method for generating a library relates to integration of binder genes into higher eukaryotic cells with larger genomes where homologous recombination in the absence of nuclease cleavage is less effective. Yeast (e.g., Saccharomyces cerevisiae) has a smaller genome than mammalian cells and homologous recombination directed by homology arms (in the absence of nuclease-directed cleavage) is an effective way of introducing foreign DNA compared to higher eukaryotes. Nuclease-directed integration has been used in yeast cells to solve the problem of efficient integration of multiple genes into individual yeast cells, e.g., for engineering of metabolic pathways (US2012/0277120), but this work does not incorporate introduction of libraries of binders nor does it address the problems of library construction in higher eukaryotes.

Preferred eukaryotic cells are T lymphocyte lineage cells (e.g., primary T cells or a T cell line) or B lymphocyte lineage cells. Of particular preference are primary T-cells or T cell derived cell lines for use in TCR libraries including cell lines which lack TCR expression [23, 24, 25]. Preferred B lymphocyte lineage cells are B cells, pre-B cells or pro-B cells and cell lines derived from any of these.

Construction of libraries in primary B cells or B cell lines would be of particular value for construction of antibody libraries. These eukaryotic cells are preferred in methods for identifying a locus and for producing a library. Breous-Nystrom et al. have generated libraries in a murine pre-B cell line (1624-5). The chicken B cell derived cell line DT40 (ATCC CRL-2111) has particular promise for construction of libraries of binders. DT40 is a small cell line with a relatively rapid rate of cell division. Repertoires of binders could be targeted to specific loci using ZFNs, TALE nucleases or CRISPR/Cas9 targeted to endogenous sequences or by targeting pre-integrated heterologous sites which could include meganuclease recognition sites. DT40 cells express antibodies and so it will be advantageous to target antibody genes within the antibody locus either with or without disruption of the endogenous chicken antibody variable domains. DT40 cells have also been used as the basis of an in vitro system for generation of chicken IgMs termed the Autonomously Diversifying Library system (ADLib system) which takes advantage of intrinsic diversification occurring at the chicken antibody locus. As a result of this endogenous diversification it is possible to generate novel specificities. The nuclease-directed approach described here could be used in combination with ADLib to combine diverse libraries of binders from heterologous sources (e.g., human antibody variable region repertoires or synthetically derived alternative scaffolds) with the potential for further diversification with the chicken IgG locus. Similar benefits could apply to human B cell lines such as Nalm6 [26].

Other preferred B lineage cell lines preferred in methods for identifying a locus and for producing a library include lines such as the murine pre-B cell line 1624-5 and the pro-B cell line Ba/F3. Ba/F3 is dependent on IL-3 and its use is discussed elsewhere herein. Finally a number of human cell lines are preferred including those listed in the “Cancer Cell Line Encyclopaedia” or “COSMIC catalogue of somatic mutations in cancer” [29].

In a method for producing a library as well as in libraries according to the invention, the eukaryotic cells are preferably of a single type of cells, produced by introduction of donor DNA into a population of clonal eukaryotic cells, for example by introduction of donor DNA into cells of a particular cell line. The main significant difference between the different library clones will then be due to integration of the donor DNA.

### Eukaryotic Viral Systems

The advantages of the aspects of the present invention, such as the methods for producing a library of eukaryotic cell clones, and the resulting libraries, could be applied to viral display systems based around eukaryotic expression systems, e.g., baculoviral display or retroviral display [1, 2, 3, 4]. In this approach each cell will encode a binder capable of being incorporated into a viral particle. In the case of retroviral systems the encoding mRNA would be packaged and the encoded binder would be presented on the cell surface. In the case of baculoviral systems, genes encoding the binder would need to be encapsulated into the baculoviral particle to maintain an association between the gene and the encoded protein. This could be achieved using host cells carrying episomal copies of the baculoviral genome. Alternatively integrated copies could be liberated following the action of a specific nuclease (distinct from the one used to drive site-specific integration). In the case of multimeric binder molecules some partners could be encoded within the cellular DNA with the genes for one or more partners being packaged within the virus.

### Introduction of Nucleic Acids

The methods described herein comprise the introduction of nucleic acids into a eukaryotic cell. In a method for identifying a locus, the landing pad sequence (i.e. a nucleic acid) is introduced, and preferably a donor DNA sequence introduced. In a method for generating a library, donor DNA molecules are introduced. Unless specifically mentioned otherwise, the introduction of a nucleic acid refers to the introduction of a DNA molecule in a eukaryotic cell.

Numerous methods have been described for introducing nucleic acids into eukaryotic cells, including transfection, infection or electroporation. Transfection of large numbers of cells is possible by standard methods including polyethyleneimine—mediated transfection as described herein. In addition methods are available for highly efficient electroporation of 10¹⁰ cells in 5 minutes, e.g., http://www.maxcyte.com. In a method for generating a library, combinatorial libraries could be created wherein members of multimeric binding pairs (e.g., VH and VL genes of antibody genes) or even different parts of the same binder molecule are introduced on different plasmids. Introduction of separate donor DNA molecules encoding separate binders or binder subunits may be done simultaneously or sequentially. For example an antibody light chain could be introduced by transfection or infection, the cells grown up and selected if necessary. Other components could then be introduced in a subsequent infection or transfection step. One or both steps could involve nuclease-directed integration to specific genomic loci.

### Integration of Nucleic Acids

A method for identifying a locus or a method for generating a library involves the integration of nucleic acids into the genome of the eukaryotic cell. In this context, the terms genome and cellular DNA may be used interchangeably. Unless explicitly mentioned otherwise, integration refers to the integration of a DNA molecule into the genome of a eukaryotic cell. The nucleic acid is integrated into the genome (i.e. cellular DNA), forming recombinant DNA having a contiguous DNA sequence in which the nucleic acid is inserted at the integration site. In the present invention, integration is mediated by the natural DNA repair mechanisms that are endogenous to the cell.

Integration of a nucleic acid may be random or specific. The random integration of a nucleic acid preferably refers to the random integration of the nucleic acid into the genome of a eukaryotic cell via transposon-mediated integration. Herein, the integration site is not defined by a specific sequence. A method for identifying a locus comprises the random integration of the landing pad sequence into the eukaryotic cell. The term “transposon” or “transposon vector” or “transposable element” is used herein as customarily and ordinarily understood by the skilled person. Transposons are genetic elements which may integrate into cellular DNA in a non-site-specific manner and, when engineered to carry or flank a landing pad sequence, cause this sequence to be inserted at a random location in the cellular DNA. The skilled person is aware of suitable transposons, many of which are commercially available, such as the PiggyBac system, an example of which is provided in the experimental section herein. The PiggyBac system is further described in for example Wilson et al. Molecular Therapy vol. 15 no. 1, 139-145 January 2007; Kim et al. Mol Cell Biochem (2011) 354:301-309); Galvan et al. Immunother. 2009 October; 32 (8): 837-844. Generally, the PiggyBac system utilizes two vectors. One vector, referred to as the helper PBase vector, encodes a transposase. The other vector, referred to as the transposon vector, contains two terminal repeats (TRs) bracketing the region to be transposed. The landing pad to be delivered into host cells may be cloned into this region using molecular techniques which are standard in the art. When the PBase vector and the PiggyBac transposon vector are co-transfected into target cells, the transposase produced from the helper recognizes the two TRs on the transposon, and inserts the flanked region including the two TRs into the host cellular DNA. Integration typically occurs at host chromosomal sites that contain a TTAA sequence, which is duplicated on the two flanks of the integrated fragment. The transposon may be integrated into the host cell's genome into a single locus (single-copy integration) or multiple loci (multiple-copy integration).

In specific integration of a nucleic acid the integration site is defined by a specific sequence. The nucleic acid in the context of specific integration may be called the donor DNA, the donor DNA molecule or the donor DNA sequence.

Specific integration can be allowed to occur by introducing the nucleic acid into a cell, allowing the site-specific nuclease to create an integration site, and allowing the donor DNA to be integrated. In this context, specific integration may also be called nuclease-directed integration. Cells may be kept in culture for sufficient time for the DNA to be integrated. This will usually result in a mixed population of cells, including (i) recombinant cells into which the donor DNA has integrated at the integration site created by the site-specific nuclease, and optionally (ii) cells in which donor DNA has integrated at sites other than the desired integration site and/or optionally (iii) cells that into which donor DNA has not integrated. The desired recombinant cells and the resulting clones may thus be provided in a mixed population further comprising other eukaryotic cells. Selection methods described elsewhere herein may be used to selected the desired cells and clones, or to enrich said mixed population in said desired cells and clones.

As explained above, integration is mediated by the natural DNA repair mechanisms that are endogenous to the cell. Endogenous DNA repair mechanisms in eukaryotic cells include homologous recombination, non-homologous end joining (NHEJ) and microhomology-directed end joining. The efficiency of integration by such processes can be increased by the introduction of double stranded breaks (DSBs) in the cellular DNA and efficiency gains of 40,000 fold have been reported using rare cutting endonucleases (meganucleases) such as I-Sce1 [48, 49, 50].

Unlike the site-specific recombination involved in systems such as the Flp-In system [16], integration in the present invention does not require exogenous recombinases or engineered recombinase recognition sites. Therefore, a method for identifying a locus and a method for generating a library preferably do not include a step of recombinase-mediated integration of a DNA molecule. Furthermore, the eukaryotic cells in a method for identifying a locus and in a method for generating a library preferably lack a recombination site for a site-specific recombinase. The mechanisms and practicalities of specific integration of donor DNA into cellular DNA by recombinases and nucleases are very distinct as discussed by Jasin 1996 [50].

In contrast specific integration comprising the use of site-specific nuclease involves the nuclease act to create breaks or nicks within the cellular DNA, which are exposed to and repaired by endogenous cellular repair mechanisms such as homologous recombination or NHEJ. Recombinase-based approaches have an absolute requirement for pre-integration of their recognition sites, so such methods require engineering of the “hot spot” integration site into the cellular DNA as a preliminary step. With nuclease-directed integration it is possible to engineer nucleases or direct via guide RNA in the case of CRISPR: Cas9 to recognise endogenous recognition sequences, i.e., nucleic acid sequences occurring naturally in the cellular DNA. Finally, at a practical level nuclease-directed approaches are more efficient for specific integration of transgenes at the levels required to make large libraries of binders.

The DNA repair mechanism by which the donor DNA is integrated in a method for identifying a locus or in a method for generating a library can be pre-determined or biased to some extent by design of the donor DNA and/or choice of site-specific nuclease.

Homologous recombination is a natural mechanism used by cells to repair double stranded breaks using homologous sequence (e.g., from another allele) as a template for repair. Homologous recombination has been utilised in cellular engineering to introduce insertions (including transgenes), deletions and point mutations into the genome. Homologous recombination is promoted by providing homology arms on the donor DNA. Hence, the donor DNA preferably comprises homology arms. The original approach to engineering higher eukaryotic cells typically used homology arms of 5-10 kb within a donor plasmid to increase efficiency of targeted integration into the site of interest. Homologous recombination is particularly suitable for eukaryotes such as yeast, which has a genome size of only 12.5×10⁶ bp, where it is more effective compared with higher eukaryotes with larger genomes e.g., mammalian cells with 3000×10⁶ bp.

Homologous recombination can also be directed through nicks in cellular DNA and this could also serve as a route for nuclease-directed integration into cellular DNA. Hence, the integration of donor DNA comprised in a method for identifying a locus or method for generating a library preferably comprised the introduction of nicks in the cellular DNA. Two distinct pathways have been shown to promote homologous recombination at nicked DNA. One is essentially similar to repair at double strand breaks, utilizing Rad51/Brca2, while the other is inhibited by Rad51/Brca2 and preferentially uses single-stranded DNA or nicked double stranded donor DNA [51].

Non-homologous end-joining (NHEJ) is an alternative mechanism to repair double stranded breaks in the genome where the ends of DNA are directly re-ligated without the need for a homologous template. Nuclease-directed cleavage of genomic DNA can also enhance transgene integration via non-homology based mechanisms. NHEJ provides a simple means of integrating in-frame exons into intron or allows integration of promoter: gene cassettes into the genome. Use of non-homologous methods allows the use of donor vectors which lack homology arms thereby simplifying the construction of donor DNA.

It has been pointed out that short regions of terminal homology are used to re-join DNA ends and it was hypothesized that 4 bp of microhomology might be utilized for directing repairing at double strand breaks, referred to as microhomology-directed end joining [50].

### Site-Specific Nuclease

The invention involves the use of site-specific nucleases and their recognition sequences. On the one hand, a method for identifying a locus comprises providing a landing pad sequence, wherein the landing pad sequence preferably comprises a recognition sequence for a site-specific nuclease. More preferably, the method comprises providing a site-specific nuclease within the cells, wherein the nuclease cleaves the recognition sequence comprised in the landing pad. On the other hand, a method for generating a library involves providing a site-specific nuclease which cleaves a recognition sequence in cellular DNA. Preferred site-specific nucleases are defined below. It is understood that all preferences relating to site-specific nuclease may also be applied mutatis mutandis to the corresponding recognition sites.

The site-specific nuclease cleaves cellular DNA following specific binding to a recognition sequence, thereby creating an integration site for donor DNA. In this context, the terms site, target site, recognition site and recognition sequence may be used interchangeably. The nuclease may create a double strand break or a single strand break (a nick). Nuclease-mediated DNA cleavage enhances site-specific integration of binder genes through endogenous cellular DNA repair mechanisms.

In a method for identifying a locus or in a method for generating a library, the eukaryotic cells used may contain endogenous sequences recognized by the site-specific nuclease or the recognition sequence may be engineered into the cellular DNA. Furthermore, the site-specific nuclease may be exogenous to the cells, i.e. not occurring naturally in cells of the chosen type.

In a method for identifying a locus or in a method for generating a library, the site-specific nuclease can be introduced before, after or simultaneously with introduction of the donor DNA. It may be convenient for the donor DNA to encode the nuclease in addition to a binder, or on separate nucleic acid which is co-transfected or otherwise introduced at the same time as the donor DNA. Clones of a library may optionally retain nucleic acid encoding the site-specific nuclease, or such nucleic acid may be only transiently transfected into the cells.

In embodiments, a method for identifying a locus according to the invention comprises a step of integrating a donor DNA into the cells, as defined elsewhere herein, wherein said step comprises providing a site-specific nuclease within the cells, wherein the nuclease cleaves the recognition sequence comprised in the landing pad.

Any suitable site-specific nuclease may be used with the invention. It may be a naturally occurring enzyme or an engineered variant. There are a number of known nucleases that are especially suitable, such as those which recognise, or can be engineered to recognise, sequences that occur only rarely in cellular DNA.

Preferably, the site-specific nuclease recognizes only one or two distinct recognition sequences. This is advantageous since this should ensure that only one or two molecules of donor DNA are integrated per cell. Rarity of the sequence recognised by the site-specific nuclease is more likely if the recognition sequence is relatively long. Preferably, the recognition sequence has a length of at least 10, 11, 12, 13, 14, 15, 16, 17, 18, 19, 20, 21, 22, 23, 24, 25, 26, 27, 28, 29, or 30 nucleotides. Preferably, the recognition sequence has a length from 10 up to 40, 39, 38, 37, 36, 35, 34, 33, 32, 31, 30, 29, 28, 27, 26, 25, 24, 23, 22, 21 or 20 nucleotides, or from 12 up to 40, 39, 38, 37, 36, 35, 34, 33, 32, 31, 30, 29, 28, 27, 26, 25, 24, 23, 22, 21 or 20 nucleotides.

Preferred site-specific nucleases are meganucleases, zinc finger nucleases (ZFNs), TALE nucleases, and nucleic acid-guided (e.g., RNA-guided) nucleases such as the CRISPR/Cas system. Each of these produces double strand breaks although engineered forms are known which generate single strand breaks. In embodiments, the landing pad sequence comprises a corresponding nuclease recognition sequence.

Meganucleases (also known as homing endonucleases) are nucleases which occur across all the kingdoms of life and recognise relatively long sequences (12-40 bp). Given the long recognition sequence they are either absent or occur relatively infrequently in eukaryotic genomes. Meganucleases are grouped into 5 families based on sequence/structure. (LAGLIDADG (SEQ ID NO: 76), GIY-YIG (SEQ ID NO: 77), HNH, His-Cys box and PD-(D/E) XK families). The best studied family is the LAGLIDADG family which includes the well characterised I-SceI meganuclease from Saccharomyces cerevisiae. I-SceI recognises and cleaves an 18 bp recognition sequence (5′ TAGGGATAACAGGGTAAT, SEQ ID NO: 70) leaving a 4 bp 3′ overhang. Another commonly used example is I-CreI which originates from the chloroplast of the unicellular green algae of Chlamydomonas reinhardtii, and recognizes a 22 bp sequence [30]. A number of engineered variants have been created with altered recognition sequences [31]. Meganucleases represent the first example of the use of site-specific nucleases in genome engineering [49, 50]. As with recombinase-based approaches, use of I-SceI and other meganucleases requires prior insertion of an appropriate recognition sequence to be targeted within the genome or engineering of meganucleases to recognize endogenous recognition sequences [30]. By this approach targeting efficiency in HEK293 cells (as judged by homology-directed “repair” of an integrated defective GFP gene) was achieved in 10-20% of cells through the use of I-SceI [32].

A preferred class of meganucleases is the LAGLIDADG endonucleases. These include I-SceI, I-ChuI, I-Cre I, CsmI, PI-SceI, PI-TliI, PI-MtuI, I-CeuI, I-SceII, I-SceIII, HO, Pi-CivI, PI-CtrI, PI-AaeI, PI-BsuI, PI-DhaI, PI-DraI, PI-MavI, PI-MchI, PI-Mfu, PI-MflI, PI-MgaI, PI-MgoI, PI-MinI, PI-MkaI, PI-MieI, PI-MrnaI, PI-MshI, PI-MsmI, PI-MthI, PI-Mtu, PI-MxeI, PI-NpuI, PI-PfuI, PI-RmaI, PI-SpbI, PI-SspI, PI-FacI, PI-MjaI, PI-PhoI, Pi-TagI, PI-ThyI, PI-Tko I, I-MsoI, and PI-TspI; preferably, I-SceI, I-CreI, I-ChuI, I-DmoI, I-CsmI, PI-SceI, PI-PfuI, PI-TliI, PI-MtuI, and I-CeuI. In embodiments, the landing pad sequence comprises a corresponding nuclease recognition sequence.

In recent years a number of methods have been developed which allow the design of novel sequence-specific nucleases by fusing sequence-specific DNA binding domains to non-specific nucleases to create designed sequence-specific nucleases directed through bespoke DNA binding domains. Binding specificity can be directed by engineered binding domains such as zinc finger domains. These are small modular domains, stabilized by Zinc ions, which are involved in molecular recognition and are used in nature to recognize DNA sequences. Arrays of zinc finger domains have been engineered for sequence specific binding and have been linked to the non-specific DNA cleavage domain of the type II restriction enzyme Fok1 to create zinc finger nucleases (ZFNs). Such ZFNs are preferred site-specific nucleases herein. ZFNs can be used to create double stranded break at specific sites within the genome. Fok1 is an obligate dimer and requires two ZFNs to bind in close proximity to effect cleavage. The specificity of engineered nucleases has been enhanced and their toxicity reduced by creating two different Fok1 variants which are engineering to only form heterodimers with each other [33]. Such obligate heterodimer ZFNs have been shown to achieve homology-directed integration in 5-18% of target cells without the need for drug selection [21, 34, 35]. Incorporation of inserts up to 8 kb with frequencies of >5% have been demonstrated in the absence of selection.

The ability to engineer DNA binding domains of defined specificity has been further simplified by the discovery in Xanathomonas bacteria of Transcription activator-like effectors (TALE) molecules. These TALE molecules consist of arrays of monomers of 33-35 amino acids with each monomer recognising a single base within a target sequence [37]. This modular 1:1 relationship has made it relatively easy to design engineered TALE molecules to bind any DNA target of interest. By coupling these designed TALEs to Fok1 it has been possible to create novel sequence-specific TALE-nucleases. TALE nucleases, also known as TALENs, are preferred site-specific nucleases in this application and have been designed to a large number of sites (i.e. recognition sequences) and exhibit high success rate for efficient gene modification activity [38]. Other variations and enhancements of TALE nuclease technology have been developed and could be used as site-specific nucleases in methods for identifying a locus or in methods for generating a library. These included “mega-TALENs” where a TALE nuclease binding domain is fused to a meganuclease and “compact TALENs” where a single TALE nuclease recognition domain is used to effect cleavage [40]. In recent years another system for directing double- or single-stranded breaks to specific sequences in the genome has been described. This system called “Clustered Regularly Interspaced Short Palindromic Repeats (CRISPR) and CRISPR Associated (Cas)” system is based on a bacterial defence mechanism [41]. The CRISPR/Cas system is a preferred site-specific nuclease in a method for identifying a locus or in a method for generating a library. The CRISPR/Cas system targets DNA for cleavage via a short, complementary single-stranded RNA (CRISPR RNA or crRNA) adjoined to a short palindromic repeat. In the commonly used “Type II” system, the processing of the targeting RNA is dependent on the presence of a trans-activating crRNA (tracrRNA) that has sequence complementary to the palindromic repeat. Hybridization of the tracrRNA to the palindromic repeat sequence triggers processing. The processed RNA activates the Cas9 domain and directs its activity to the complementary sequence within DNA. The system has been simplified to direct Cas9 cleavage from a single RNA transcript and has been directed to many different sequences within the genome [42, 43]. This approach to genome cleavage has the advantage of being directed via a short RNA sequence making it relatively simple to engineer cleavage specificity. Thus there are a number of different ways to achieve site-specific cleavage of genomic DNA. As described above this enhances the rate of integration of a donor plasmid through endogenous cellular DNA repair mechanisms.

In a method for generating a library, use of meganucleases, ZFNs, TALE nuclease or nucleic acid guided systems such as the CRISPR/Cas9 systems as site-specific nucleases will enable targeting of endogenous loci within the genome.

Alternatively, in a method for identifying a locus and in a method for generating a library, heterologous recognition sites (i.e. recognition sequences) for site-specific nucleases, including meganucleases, ZFNs and TALE nucleases could be introduced in advance. Nuclease-directed targeting could be used to drive insertion of recognition sequences by homologous recombination or NHEJ using vector DNA or even double stranded oligonucleotides [45]. As an alternative, non-specific targeting methods could be used to introduce recognition sequences for site-specific nucleases through the use of transposon-directed integration [46]. Viral-based systems, such as lentivirus, applied at low titre could also be used to introduce recognition sequences.

The site-specific nuclease may be encoded by a single gene that is introduced on one plasmid, whereas the donor DNA is present on a second plasmid. Of course, combinations could be used incorporating two or more of these elements on the same plasmid and this could enhance the efficiency of targeting by reducing the number of number of plasmids to be introduced in a method for identifying a locus or a method for generating a library. In addition it may be possible to pre-integrate the nuclease(s) which could also be inducible to allow temporal control of nuclease activity as has been demonstrated for transposases [46].

Finally the nuclease could be introduced as recombinant protein or protein: RNA complex (for example in the case of an RNA directed nuclease such as CRISPR: Cas9).

### Recognition Sequences

As noted, a method for generating a library involves providing a site-specific nuclease which cleaves a recognition sequence in cellular DNA.

In some embodiments, the recognition sequence is in a neurolysin (NLN) gene. The eukaryotic cells used may contain endogenous sequences recognized by the site-specific nuclease or the recognition sequence may be engineered into the cellular DNA as earlier described herein. The neurolysin gene (human sequence: Uniprot Q9BYT8, ENSEMBL gene id ENSG00000123213) encodes a member of the metallopeptidase M3 protein family that cleaves neurotensin at the Pro10-Tyr11 bond, leading to the formation of neurotensin (1-10) and neurotensin (11-13). An exemplary sequence of a neurolysin gene is represented by SEQ ID NO: 1. In some embodiments, the recognition sequence is in a nucleic acid molecule represented by a nucleotide sequence comprising, consisting essentially of, or consisting of SEQ ID NO: 1, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 1.

In some embodiments, the recognition sequence is in a TRAF2 and NCK interacting kinase (TNIK) gene (Uniprot Q9UKE5, ENSEMBL gene id ENSG00000154310). An exemplary sequence of a TNIK gene is represented by SEQ ID NO: 2. In some embodiments, the recognition sequence is in a protein mono-ADP-ribosyltransferase 11 (PARP11) gene (Uniprot Q9NR21, ENSEMBL gene id ENSG00000111224). An exemplary sequence of a PARP11 gene is represented by SEQ ID NO: 3. In some embodiments, the recognition sequence is in a RAB40B gene (member RAS oncogene family, Uniprot Q12829, ENSEMBL gene id ENSG00000141542). An exemplary sequence of a RAB40B gene is represented by SEQ ID NO: 4. In some embodiments, the recognition sequence is in an abl interactor 2 (ABI2) gene (Uniprot Q9NYB9, ENSEMBL gene id ENSG00000138443). An exemplary sequence of an ABI2 gene is represented by SEQ ID NO: 5. In some embodiments, the recognition sequence is in a ring finger protein 19B (RNF19B) gene (Uniprot Q6ZMZO, ENSEMBL gene id ENSG00000116514). An exemplary sequence of an RNF19B gene is represented by SEQ ID NO: 6. In some embodiments, the recognition sequence is in a cAMP-dependent protein kinase inhibitor alpha (PKIA) gene (Uniprot P61925, ENSEMBL gene id ENSG00000171033). An exemplary sequence of a PKIA gene is represented by SEQ ID NO: 7. In some embodiments, the recognition sequence is in a formimidoyltransferase cyclodeaminase (FTCD) gene (Uniprot O95954, ENSEMBL gene id ENSG00000160282). An exemplary sequence of an FTCD gene is represented by SEQ ID NO: 8.

In some embodiments, the recognition sequence is in an NLN gene, a TNIK gene or a RAB40B gene In some embodiments, the recognition sequence is in a nucleic acid molecule represented by a nucleotide sequence comprising, consisting essentially of, or consisting of SEQ ID NOs: 1-8, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 1-8.

In some embodiments, the recognition sequence is in an intron of a gene selected from an NLN, TNIK, PARP11, RAB40B, ABI2, RNF19B, PKIA, or FTCD gene, preferably an NLN, TNIK, or RAB40B genes. An intron is used herein as customarily and ordinarily understood by the skilled person.

A recognition sequence in an intron of NLN is preferably in NLN-207 intron 1 (intron 1-2), intron 2 (intron 2-3) or intron 6 (intron 6-7). A recognition sequence in an intron of TNIK is preferably in TNIK-04 (Ensembl ID ENST00000436636.7) intron 2 (intron 2-3). A recognition sequence in an intron of PARP11 is preferably in PARP11-205 (Ensembl ID ENST00000450737.2) intron 1 (intron 1-2). A recognition sequence in an intron of RAB40B is preferably in RAB40B-206 (Ensembl ID ENST00000571995.6) intron 1 (intron 1-2). A recognition sequence in an intron of ABI2 is preferably in ABI2-203 (Ensembl ID ENST00000261018.12) intron 1 (intron 1-2). A recognition sequence in an intron of RNF19B is preferably in RNF19B-201 (Ensembl ID ENST00000235150.5) intron 1 (intron 1-2). A recognition sequence in an intron of PKIA is preferably in PKIA-202 (Ensembl ID ENST00000396418.7) intron 1 (intron 1-2). A recognition sequence in an intron of FTCD is preferably in FTCDNL1-201 (Ensembl ID ENST00000416668.5) intron 3 (intron 3-4).

In preferred embodiments, the recognition sequence is in an intron of a neurolysin gene. The canonical transcript of the human neurolysin (NLN) gene is NLN-201 (Ensembl transcript ID: ENST00000380985.10) which comprises 13 exons. An alternative transcript is NLN-207 (Ensembl transcript ID: ENST00000509935.2) which comprises 7 exons. In some embodiments, the recognition sequence is in NLN-201 intron 1 of a neurolysin gene (NLN-201 intron 1-2; exemplary sequence: SEQ ID NO: 9). In some embodiments, the recognition sequence is in NLN-201 intron 2 of a neurolysin gene (NLN-201 intron 2-3; exemplary sequence: SEQ ID NO: 10). In some embodiments, the recognition sequence is in NLN-201 intron 3 of a neurolysin gene (NLN-201 intron 3-4; exemplary sequence: SEQ ID NO: 11). In some embodiments, the recognition sequence is in NLN-201 intron 4 of a neurolysin gene (NLN-201 intron 4-5; exemplary sequence: SEQ ID NO: 12). In some embodiments, the recognition sequence is in NLN-201 intron 5 of a neurolysin gene (NLN-201 intron 5-6; exemplary sequence: SEQ ID NO: 13). In some embodiments, the recognition sequence is in NLN-201 intron 6 of a neurolysin gene (NLN-201 intron 6-7; exemplary sequence: SEQ ID NO: 14). In some embodiments, the recognition sequence is in NLN-201 intron 7 of a neurolysin gene (NLN-201 intron 7-8; exemplary sequence: SEQ ID NO: 15). In some embodiments, the recognition sequence is in NLN-201 intron 8 or NLN-207 intron 1 of a neurolysin gene (NLN-201 intron 8-9 or NLN-207 intron 1-2; exemplary sequence: SEQ ID NO: 16). In some embodiments, the recognition sequence is in NLN-201 intron 9 or NLN-207 intron 2 of a neurolysin gene (NLN-201 intron 9-10 or NLN-207 intron 2-3; exemplary sequence: SEQ ID NO: 17). In some embodiments, the recognition sequence is in NLN-201 intron 10 or NLN-207 intron 3 of a neurolysin gene (NLN-201 intron 10-11 or NLN-207 intron 3-4; exemplary sequence: SEQ ID NO: 18). In some embodiments, the recognition sequence is in NLN-201 intron 11 or NLN-207 intron 4 of a neurolysin gene (NLN-201 intron 11-12 or NLN-207 intron 4-5; exemplary sequence: SEQ ID NO: 19). In some embodiments, the recognition sequence is in intron 12 of a NLN-201 neurolysin gene (NLN-201 intron 12-13; exemplary sequence: SEQ ID NO: 20). In some embodiments, the recognition sequence is in intron 5 of a NLN-207 neurolysin gene (NLN-207 intron 5-6; exemplary sequence: SEQ ID NO: 21). In some embodiments, the recognition sequence is in intron 6 of a NLN-207 neurolysin gene (NLN-207 intron 6-7; exemplary sequence: SEQ ID NO: 22).

Preferred introns are NLN-207 introns 1, 2, and 6 of an NLN gene. In some embodiments, the recognition sequence is in a nucleic acid molecule represented by a nucleotide sequence comprising, consisting essentially of, or consisting of SEQ ID NOs: 16, 17, 22, or a nucleotide sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NOs: 16, 17, 22.

Preferably, the recognition sequence comprises, consists essentially of, or consists of SEQ ID NO: 15, or a nucleotide sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 23.

In some embodiments, particularly when the recognition sequence is in an intron of a gene selected from an NLN, TNIK, PARP11, RAB40B, ABI2, RNF19B, PKIA, or FTCD gene as described above, the recognition sequence is in an open chromatin region of the intron.

In some embodiments, particularly when the recognition sequence is in an intron of a gene selected from an NLN, TNIK, PARP11, RAB40B, ABI2, RNF19B, PKIA, or FTCD gene as described above, the recognition sequence is in an enhancer region of the intron.

As used herein “open chromatin” or “euchromatin” or “loose chromatin” refers to a structure that is permissible for transcription whereas “heterochromatin” or “tight” or “closed” chromatin is more compact and more refractory to factors that need to gain access to the DNA template.

### Distribution of Recognition Sequences

A recognition sequence for the site-specific nuclease in a method according to the invention may be present in genomic DNA, or episomal DNA which is stably inherited in the cells. Donor DNA may therefore be integrated at a genomic or episomal locus in the cellular DNA. Preferably, a genomic locus is identified via a method for identifying a locus.

In its simplest form a single gene encoding a binder (binder gene) is targeted to a single site within the eukaryotic genome. Identification of a cell demonstrating a particular binding activity or cellular phenotype will allow direct isolation of the gene encoding the desired property (e.g., by PCR from mRNA or genomic DNA). This is facilitated by using a unique recognition sequence for the site-specific nuclease, occurring once in the cellular DNA. Cells used for creation of the library may thus contain a nuclease recognition sequence at a single fixed locus, i.e., one identical locus in all cells. Libraries produced from such cells will contain donor DNA integrated at the fixed locus, i.e., occurring at the same locus in cellular DNA of all clones in the library.

Optionally, recognition sequences may occur multiple times in cellular DNA, so that the cells have more than one potential integration site for donor DNA. This would be a typical situation for diploid or polyploid cells where the recognition sequence is present at corresponding positions in a pair of chromosomes, i.e., replicate loci. Libraries produced from such cells may contain donor DNA integrated at replicate fixed loci. For example libraries produced from diploid cells may have donor DNA integrated at duplicate fixed loci and libraries produced from triploid cells may have donor DNA integrated at triplicate fixed loci. Many suitable mammalian cells are diploid, and clones of mammalian cell libraries according to the invention may have donor DNA integrated at duplicate fixed loci.

The sequence recognised by the site-specific nuclease may occur at more than one independent locus in the cellular DNA. Donor DNA may therefore integrate at multiple independent loci. Libraries of diploid or polyploid cells may comprise donor DNA integrated at multiple independent fixed loci and/or at replicate fixed loci.

In cells containing recognition sequences at multiple loci (whether replicate or independent loci), each locus represents a potential integration site for a molecule of donor DNA. Introduction of donor DNA into the cells may result in integration at the full number of nuclease recognition sequences present in the cell, or the donor DNA may integrate at some but not all of these potential sites. For example, when producing a library from diploid cells containing recognition sequences at first and second fixed loci (e.g., duplicate fixed loci), the resulting library may comprise clones in which donor DNA is integrated at the first fixed locus, clones in which donor DNA is integrated at the second fixed locus, and clones in which donor DNA is integrated at both the first and second fixed loci.

Methods of producing libraries may therefore involve site-specific nuclease cleavage of multiple fixed loci in a cell, and integration of donor DNA at the multiple fixed loci. As noted above, in cases where there are multiple copies of the same recognition sequence (e.g., as occurs when targeting endogenous loci in diploid or polyploid cells) it is possible that two binder genes will be integrated, particularly when an efficient targeting mechanisms is used, with only one gene being specific to the target. This can be resolved during subsequent screening once binder genes have been isolated.

In some instances it may be desirable to introduce more than one binder per cell. For example bi-specific binders could be generated from two different antibodies coming together and these may have properties absent in the individual binders [47]. This could be achieved by introducing different antibody genes into both alleles at duplicate fixed loci or by targeting different antibody populations into independent fixed loci using the methods described herein. Furthermore a binder may itself be composed of multiple chains (e.g., antibody VH and VL domains presented within a Fab or IgG format). In this case it may be desirable to integrate the different sub-units into different loci. These could be integrated within the same cycle of nuclease-directed integration, they could be integrated sequentially using nuclease-directed integration for one or both integration steps.

### Landing Pad Sequence

In step a) of the method for identifying a locus, a landing pad sequence is provided. As used herein, a “landing pad sequence” may be understood to refer to a nucleotide sequence directing the integration or “landing” of a donor DNA molecule at a specific genomic locus. A landing pad sequence generally comprises a nucleotide sequence recognized by a site-specific recombinase or site-specific nuclease (“recognition sequence”) allowing site-specific recombinase-directed or nuclease-directed integration of a donor DNA molecule comprising one or more transgenes of interest, for example a transgene encoding a binder or a selectable marker as described later herein. In embodiments, the landing pad sequence comprises a recognition sequence for a site-specific nuclease. Preferred recognition sequences are defined elsewhere herein.

Optionally, a landing pad sequence may comprise additional nucleotide sequences which facilitate screening and/or selection of clones having integrated the landing pad sequence into their genome, such as a selectable marker like a gene conferring resistance to an antibiotic. A landing pad sequence may optionally further comprise nucleotide sequences which facilitate the screening and/or selection of clones having integrated a donor DNA sequence into the landing pad sequence, such as a promoter or other regulatory region. As a non-limiting example, a promoter flanking a site-specific nuclease recognition site in a landing pad sequence may be operably linked to a promoterless transgene of interest following genomic integration of the transgene after cellular DNA cleavage by the site-specific nuclease. The resulting transgene expression may then be used for screening and/or selection purposes.

Selection of Clones with Integrated Landing Pad Sequence

Step d) of the method for identifying a locus involves selecting a clone having a landing pad sequence integrated into its genome. In cases wherein the landing pad sequence comprises a selectable marker like a gene conferring resistance to an antibiotic (such as blasticidin or puromycin), clones may be selected via culturing the cells in the presence of the antibiotic. Alternatively, clones may be screened and/or selected using standard molecular toolbox methods in the art, such as Southern Blotting or PCR. The selection of clones may comprise screening the clones. For example, inverted PCR (iPCR), as described in Schuldiner et al. (2018) Dev Cell 14:227-238 (incorporated herein by reference in its entirety) may be used for mapping the insertion site of transposable elements. Alternatively, splinkerette PCR (spPCR), as described in Potter and Luo (2010) PLOS ONE 5 (4): e1016 (incorporated herein by reference in its entirety) may be used. Splinkerette PCR involves the digestion of genomic DNA to yield overhanding sticky ends. The restriction enzyme is not required to cut within the landing pad sequence. Onto the sticky end is ligated a double stranded oligonucleotide (the splinkerette) that is unphosphorylated and contains a stable hairpin loop and compatible sticky end. Two rounds of nested PCR are then performed to amplify the genomic sequence between the transposon insertion site and the anneal splinkerette. This is followed by sequencing of the PCR products, using for example Sanger sequencing with another nested primer, or any other nucleic acid sequencing method known to the skilled person. Examples include Sanger sequencing, single-molecule real-time sequencing, ion torrent sequencing, pyrosequencing, Illumina-sequencing, combinatorial probe anchor synthesis, sequencing by ligation (SOLID sequencing), Nanopore sequencing, GenapSys sequencing, and the like. Sequencing sample preparation, instruments, and protocols are discussed in standard handbooks like Head, Ordoukhanian and Salomon (Eds), Next Generation Sequencing: Methods and Protocols, Humana Press, NJ, USA (2018), incorporated herein by reference in its entirety, with many being commercially available, e.g. from Illumina (CA, USA), Pacific Biosciences (CA, USA), and others. Using the above described screening and/or selection methods, clones having only a single copy of the transposon element, and thereby the landing pad sequence, integrated into their genome may be selected. Alternatively, screening for single-copy integration may be performed using whole-genome-sequencing (WGS) followed by genome assembly using standard bioinformatics tools available in the art. Alternatively, screening for single-copy integration may be performed by quantification of the expression of a transgene of interest following its integration into the landing pad sequence. Expression may be evaluated on the level of mRNA or protein by standard assays known to the person of skill in the art (e.g. qPCR, Western blotting, ELISA). Expression may be also evaluated using spectroscopic methods such as fluorescence-activated cell sorting (FACS) using commercially available devices. As a non-limiting example, a transgene encoding for a cell membrane-bound binder may be integrated into the landing pad following integration of the landing pad into the cellular DNA. Fluorescent-labelled antibodies against the binder can then be used in conjunction with FACS to quantify expression levels and select clones with single-copy integration of the binder. An example of FACS-based single-copy integration screening and selection is further provided in the experimental section herein.

These clones are particularly useful, as they can be used for the construction of libraries characterized by a uniform integration and/or uniform transcription of binders, as elsewhere described herein. In embodiments, a method for identifying a locus comprises the further steps of (e) screening for single-copy integration (of the landing pad sequence) and (f) identifying the locus (at which the landing pad sequence was integrated). Step (f) may be performed by any of the sequencing methods described above.

### Locus and Use

A “locus” in the context of a method for identifying a locus in a genome of a eukaryotic cell refers to a genomic locus which is a candidate for insertion of binder sequences. Hence, a locus identified via such a method may be used to build a library according to the invention by integrating a donor DNA sequence comprising one or more transgenes encoding a binder at the locus. Preferably, in such a use, the donor DNA is integrated at a landing pad sequence at the locus.

In a preferred embodiment, a use of a locus according to the invention comprises:
identifying a locus via a method for identifying a locus according to the invention;
providing donor DNA molecules encoding the binders, and eukaryotic cells;
introducing the donor DNA into the cells and providing a site-specific nuclease within the cells, wherein the nuclease cleaves a recognition sequence in cellular DNA, wherein the recognition sequence is at said locus, to create an integration site at which the donor DNA becomes integrated into the cellular DNA, integration occurring through DNA repair mechanisms endogenous to the cells, thereby creating recombinant cells containing donor DNA integrated in the cellular DNA; and
culturing the recombinant cells to produce clones, thereby providing a library of eukaryotic cell clones containing donor DNA encoding the repertoire of binders.

All preferred embodiments in the context of a method for producing a library according to the invention apply mutatis mutandis to a method according to this preferred embodiment.

### Donor DNA

A method for generating a library, and preferably also a method for identifying a locus, comprises integrating a donor DNA. A preferred donor DNA is described in this section.

The donor DNA will usually be circularised DNA, and may be provided as a plasmid or vector. Linear DNA is another possibility. Donor DNA molecules may comprise regions that do not integrate into the cellular DNA, in addition to one or more donor DNA sequences that integrate into the cellular DNA. The DNA is typically double-stranded, although single-stranded DNA may be used in some cases. The donor DNA contains one or more transgenes encoding a binder, for example it may comprise a promoter: gene cassette. In the simplest format double-stranded, circular plasmid DNA can be used to drive homologous recombination. This requires regions of DNA flanking the transgenes which are homologous to DNA sequence flanking the cleavage site in genomic DNA. Linearised double-stranded plasmid DNA or PCR product or synthetic genes could be used to drive both homologous recombination and NHEJ repair pathways. As an alternative to double-stranded DNA it is possible to use single-stranded DNA to drive homologous recombination [52]. A common approach to generating single-stranded DNA is to include a single-stranded origin of replication from a filamentous bacteriophage into the plasmid. Single-stranded DNA viruses such as adeno-associated virus (AAV) have been used to drive efficient homologous recombination where the efficiency has been shown to be improved by several orders of magnitude [53, 54]. Systems such as the AAV systems could be used in conjunction with nuclease-directed cleavage in a method for identifying a locus and in a method for generating a library. The benefits of both systems could be applied to in a method for identifying a locus and in a method for generating a library. The packaging limit of AAV vectors is 4.7 kb but the use of nuclease digestion of target genomic DNA will reduce this allowing larger transgene constructs to be incorporated.

A molecule of donor DNA may encode a single binder or multiple binders. Optionally, multiple subunits of a binder may be encoded per molecule of donor DNA. In some embodiments, donor DNA encodes a subunit of a multimeric binder.

In embodiments, a method for identifying a locus according to the invention comprises the additional steps of (g) integrating a donor DNA sequence comprising one or more transgenes encoding a binder at the landing pad sequence and (h) screening for integration of the donor DNA. In more embodiments, step (g) comprises providing a site-specific nuclease within the cells, wherein the nuclease cleaves the recognition sequence comprised in the landing pad. In more preferred embodiments, step (h) comprises screening for display of the one or more binders encoded by the donor DNA.

Promoters in Donor DNA and Selection of Clones with Integrated Donor DNA

In a method for identifying a locus and in a method for generating a library, the donor DNA comprises one or more transgenes encoding a binder. Transcription of the binder from the encoding donor DNA will usually be achieved by placing the sequence encoding the binder under control of a promoter and optionally one or more enhancer elements for transcription. A promoter (and optionally other genetic control elements) may be included in the donor DNA molecule itself. Alternatively, the sequence encoding the binder may lack a promoter on the donor DNA, and instead may be placed in operable linkage with a promoter on the cellular DNA, e.g., an endogenous promoter or a pre-integrated exogenous promoter, as a result of its insertion at the integration site created by the site-specific nuclease.

Donor DNA may further comprise one or more further coding sequences, such as genetic elements enabling selection of cells containing or expressing the donor DNA. Such an element may be called a selectable marker. As with the sequence encoding the binder, discussed above, such elements may be associated with a promoter on the donor DNA or may be placed under control of a promoter as a result of integration of the donor DNA at a fixed locus. The latter arrangement provides a convenient means of selecting specifically for those cells which have integrated the donor DNA at the desired site, since these cells should express the genetic element for selection. This may be, for example, a gene conferring resistance to a negative selection agent such as blasticidin or puromycin. One or more selection steps may be applied to remove unwanted cells, such as cells that lack the donor DNA or which have not integrated the donor DNA at the correct position.

The expression of a membrane anchored binder could itself be used as a form of selectable marker. For example if a library of antibody genes, formatted as IgG or scFv-Fc fusions are introduced, then cells which express the antibody can be selected using secondary reagents which recognise the surface expressed Fc using methods described herein. Upon initial transfection with donor DNA encoding the transgene under the control of an exogenous promoter, transient expression (and cell surface expression) of the binder will occur and it will be necessary to wait for transient expression to abate (to achieve targeted integration of e.g., 1-2 antibody genes/cell).

As an alternative a construct encoding a membrane tethering element (e.g., the Fc domain of the present example fused to the PDGF receptor transmembrane domain) could be pre-integrated before the binders sequences are introduced. If this membrane-tethering element lacks a promoter or is encoded within an exon which is out of frame with the preceding exon then surface expression will be compromised. Targeted integration of an incoming donor molecule can then correct this defect (e.g., by targeting a promoter or an “in-frame” exon into the intron which is upstream of the defective tethering element). If the frame “correcting exon” also encodes a binder then a fusion will be produced between the binder and the membrane tethering element resulting in surface expression of both. Thus correctly targeted integration will result in-frame expression of the membrane tethering element alone or as part of a fusion with the incoming binder. Furthermore if the incoming library of binders lack a membrane tethering element and these are incorrectly integrated they will not be selected. Thus expression of the binder itself on the cell surface can be used to select the population of cells with correctly targeted integration.

### Number of Clones and Library Diversity

A locus identified via a method for identifying a locus may be used for building a library of eukaryotic cell clones containing DNA encoding a diverse repertoire of binders. Likewise, a method for generating a library is for generating a library of eukaryotic cell clones containing DNA encoding a diverse repertoire of binders. In the context of this application, a library refers to a library of eukaryotic cell clones containing DNA encoding a diverse repertoire of binders which may be obtained via one of these methods, unless explicitly mentioned otherwise. Preferred libraries and their properties are defined in this section.

Yeast display libraries of 107-1010 have previously been constructed and demonstrated to yield binders in the absence of immunisation or pre-selection of the population [9, 55, 56, 57]. Many of the previously published mammalian display libraries used antibody genes derived from immunised donors or even enriched antigen-specific B lymphocytes, given the limitations of library size and variability when using cells from higher eukaryotes. Thanks to the efficiency of gene targeting in the methods of the current invention large, naive libraries can be constructed in higher eukaryotes such as mammalian cells, which match those described for simpler eukaryotes such as yeast.

Following integration of donor DNA into the cellular DNA, the resulting recombinant cells are cultured to allow their replication, generating a clone of cells from each initially-produced recombinant cell. Each clone is thus derived from one original cell into which donor DNA was integrated at an integration site created by the site-specific nuclease. Methods according to the present invention are associated with a high efficiency and high fidelity of donor DNA integration, and a library according to the present invention may contain at least 100, 10³, 10⁴, 10⁵, 10⁶, 10⁷,

10⁸, 10⁹ or 10¹⁰ clones.

Without being bound to this theory, using nuclease-directed integration it is possible to target 10% or more of transfected mammalian cells. It is also practical to grow and transform >10¹⁰ cells (e.g. from 5 litres of cells growing at 2×10⁶ cells/ml). Transfection of such large numbers of cells could be done using standard methods including polyethyleneimine—mediated transfection as described herein. In addition methods are available for highly efficient electroporation of 10¹⁰ cells in 5 minutes e.g. http://www.maxcyte.com. Thus using the approach of the present invention it is possible to create libraries in excess of 109 clones. When the population of donor DNA molecules that is used to create the library contains multiple copies of the same sequence, two or more clones may be obtained that contain DNA encoding the same binder. It can also be the case that a clone may contain donor DNA encoding more than one different binder, for example if there is more than one recognition sequence for the site-specific nuclease, as detailed elsewhere herein. Thus, the diversity of the library, in terms of the number of different binders encoded or expressed, may be different from the number of clones obtained.

Clones in the library preferably contain donor DNA encoding one or two members of the repertoire of binders and/or preferably express only one or two members of the repertoire of binders. A limited number of different binders per cell is an advantage when it comes to identifying the clone and/or DNA encoding a particular binder identified when screening the library against a given target. This is simplest when clones encode a single member of the repertoire of binders. However it is also straightforward to identify the relevant encoding DNA for a desired binder if a clone selected from a library encodes a small number of different binders, for example a clone may encode two members of the repertoire of binders. As discussed elsewhere herein, clones encoding one or two binders are particularly convenient to generate by selecting a recognition sequence for the site-specific nuclease that occurs once per chromosomal copy in a diploid genome, as diploid cells contain duplicate fixed loci, one on each chromosomal copy, and the donor DNA may integrate at one or both fixed loci. Thus, clones of the library may each express only one or two members of the repertoire of binders.

Binders displayed on the surface of cells of the library may be identical to (having the same amino acid sequence as) other binders displayed on the same cell. The library may consist of clones of cells which each display a single member of the repertoire of binders, or of clones displaying a plurality of members of the repertoire of binders per cell. Alternatively a library may comprise some clones that display a single member of the repertoire of binders, and some clones that display a plurality of members (e.g., two) of the repertoire of binders.

Accordingly, a library according to the present invention may comprise clones encoding more than one member of the repertoire of binders, wherein the donor DNA is integrated at duplicate fixed loci or multiple independent fixed loci.

As noted above, it is easiest to identify the corresponding encoding DNA for a binder if the corresponding clone expresses only one binder. Typically, a molecule of donor DNA will encode a single binder. The binder may be multimeric so that a molecule of donor DNA includes multiple genes or open reading frames corresponding to the various subunits of the multimeric binder.

A library according to the present invention may encode at least 100, 10³, 10⁴, 10⁵ or 10⁶, 10⁷, 10⁸, 10⁹ or 10¹⁰ different binders. Where the binders are multimeric, diversity may be provided by one or more subunits of the binder. Multimeric binders may combine one or more variable subunits with one or more constant subunits, where the constant subunits are the same (or of more limited diversity) across all clones of the library. In generating libraries of multimeric binders, combinatorial diversity is possible where a first repertoire of binder subunits may pair with any of a second repertoire of binder subunits.

### Characteristics and Form of the Library

Methods according to the invention enable construction of eukaryotic cell libraries having many advantageous characteristics. The libraries preferably have any one or more of the following features:
1. Diversity. A library may encode and/or express at least 100, 10³, 10⁴, 10⁵, 10⁶, 10⁷, 10⁸ or 10⁹ different binders.
2. Uniform integration. A library may consist of clones containing donor DNA integrated at a fixed locus, or at a limited number of fixed loci in the cellular DNA. Each clone in the library therefore contains donor DNA at the fixed locus or at least one of the fixed loci. Preferably clones contain donor DNA integrated at one or two fixed loci in the cellular DNA. As explained elsewhere herein, the integration site is at a recognition sequence for a site-specific nuclease. Integration of donor DNA to produce recombinant DNA is described in detail elsewhere herein and can generate different results depending on the number of integration sites. Where there is a single potential integration site in cells used to generate the library, the library will be a library of clones containing donor DNA integrated at the single fixed locus. All clones of the library therefore contain the binder genes at the same position in the cellular DNA. Alternatively where there are multiple potential integration sites, the library may be a library of clones containing donor DNA integrated at multiple and/or different fixed loci. Preferably, each clone of a library contains donor DNA integrated at a first and/or a second fixed locus. For example a library may comprise clones in which donor DNA is integrated at a first fixed locus, clones in which donor DNA is integrated at a second fixed locus, and clones in which donor DNA is integrated at both the first and second fixed loci. In preferred embodiments there are only one or two fixed loci in the clones in a library, although it is possible to integrate donor DNA at multiple loci if desired for particular applications. Therefore in some libraries each clone may contain donor DNA integrated at any one or more of several fixed loci, e.g., three, four, five or six fixed loci. For libraries containing binder subunits integrated at separate sites, clones of the library may contain DNA encoding a first binder subunit integrated at a first fixed locus and DNA encoding a second binder subunit integrated at a second fixed locus, wherein the clones express multimeric binders comprising the first and second subunits.
3. Uniform transcription. Relative levels of transcription of the binders between different clones of the library is kept within controlled limits due to donor DNA being integrated at a controlled number of loci, and at the same locus in the different clones (fixed locus). Relatively uniform transcription of binder genes leads to comparable levels of expression of binders on or from clones in a library. Binders displayed on the surface of cells of the library may be identical to (having the same amino acid sequence as) other binders displayed on the same cell. The library may consist of clones of cells which each display a single member of the repertoire of binders, or of clones displaying a plurality of members of the repertoire of binders per cell. Alternatively a library may comprise some clones that display a single member of the repertoire of binders, and some clones that display a plurality of members (e.g., two) of the repertoire of binders. Preferably clones of a library express one or two members of the repertoire of binders. For example, a library of eukaryotic cell clones according to the present invention may express a repertoire of at least 10³, 10⁴, 10⁵ 10⁶, 10⁷, 10⁸ or 10⁹ different binders, e.g., IgG, Fab, scFv or scFv-Fc antibody fragments, each cell containing donor DNA integrated at a fixed locus in the cellular DNA. The donor DNA encodes the binder and may further comprise a genetic element for selection of cells into which the donor DNA is integrated at the fixed locus. Cells of the library may contain DNA encoding an exogenous site-specific nuclease.

These and other features of libraries are further described elsewhere herein.

The present invention extends to the library either in pure form, as a population of library clones in the absence of other eukaryotic cells, or mixed with other eukaryotic cells. Other cells may be eukaryotic cells of the same type (e.g., the same cell line) or different cells. Further advantages may be obtained by combining two or more libraries according to the present invention, or combining a library according to the invention with a second library or second population of cells, either to facilitate or broaden screening or for other uses as are described herein or which will be apparent to the skilled person.

A library according to the invention, one or more clones obtained from the library, or host cells into which DNA encoding a binder from the library has been introduced, may be provided in a cell culture medium. The cells may be cultured and then concentrated to form a cell pellet for convenient transport or storage.

Libraries will usually be provided in vitro. The library may be in a container such as a cell culture flask containing cells of the library suspended in a culture medium, or a container comprising a pellet or concentrated suspension of eukaryotic cells comprising the library. The library may constitute at least 75%, 80%, 85% or 90% of the eukaryotic cells in the container.

It is understood that the fixed locus where the donor DNA is integrated for the libraries of the invention corresponds with the location of the recognition sequence of methods for generating a library according to the invention. Thus, all preferences for the location of the recognition sequences as described herein are also applicable to the fixed locus of the library of the invention.

### Binders

A “binder” in accordance with the present invention is a binding molecule, representing a specific binding partner for another molecule. Typical examples of specific binding partners are antibody-antigen and receptor-ligand.

The repertoire of binders encoded by a library will usually share a common structure and have one or more regions of diversity. The library therefore enables selection of a member of a desired structural class of molecules, such as a peptide or a scFv antibody molecule. For example, the binders may be polypeptides sharing a common structure and having one or more regions of amino acid sequence diversity.

This can be illustrated by considering a repertoire of antibody molecules. These may be antibody molecules of a common structural class, e.g., IgG, Fab, scFv-Fc or scFv, differing in one or more regions of their sequence. Antibody molecules typically have sequence variability in their complementarity determining regions (CDRs), which are the regions primarily involved in antigen recognition. A repertoire of binders in the present invention may be a repertoire of antibody molecules which differ in one or more CDRs, for example there may be sequence diversity in all six CDRs, or in one or more particular CDRs such as the heavy chain CDR3 and/or light chain CDR3.

Antibody molecules and other binders are described in more detail elsewhere herein. The potential of the present invention however extends beyond antibody display to include display of libraries of peptides or engineered proteins, including receptors, ligands, individual protein domains and alternative protein scaffolds [58, 59]. Nuclease-directed site-specific integration can be used to make libraries of other types of binders previously engineered using other display systems. Many of these involve monomeric binding domains such as DARPins and lipocalins, affibodies and adhirons [58, 59, 152]. Display on eukaryotes, particularly mammalian cells, also opens up the possibility of isolating and engineering binders or targets involving more complex, multimeric targets. For example T cell receptors (TCRs) are expressed on T cells and have evolved to recognise peptide presented in complex with MHC molecules on antigen presenting cells. Libraries encoding and expressing a repertoire of TCRs may be generated, and may be screened to identify binding to MHC peptide complexes as further described elsewhere herein.

For multimeric binders, donor DNA encoding the binder may be provided as one or more DNA molecules. For example, where individual antibody VH and VL domains are to be separately expressed, these may be encoded on separate molecules of donor DNA. The donor DNA integrates into the cellular DNA at multiple integration sites, e.g., the binder gene for the VH at one locus and the binder gene for the VL at a second locus. Methods of introducing donor DNA encoding separate binder subunits are described in more detail elsewhere herein. Alternatively, both subunits or parts of a multimeric binder may be encoded on the same molecule of donor DNA which integrates at a fixed locus.

A binder may be an antibody molecule or a non-antibody protein that comprises an antigen-binding site. An antigen binding site may be provided by means of arrangement of peptide loops on non-antibody protein scaffolds such as fibronectin or cytochrome B etc., or by randomising or mutating amino acid residues of a loop within a protein scaffold to confer binding to a desired target [60, 61, 62]. Protein scaffolds for antibody mimics are disclosed in WO/0034784 in which proteins (antibody mimics) are described that include a fibronectin type III domain having at least one randomised loop. A suitable scaffold into which to graft one or more peptide loops, e.g., a set of antibody VH CDR loops, may be provided by any domain member of the immunoglobulin gene superfamily. The scaffold may be a human or non-human protein.

Use of antigen binding sites in non-antibody protein scaffolds has been reviewed previously [63]. Typical are proteins having a stable backbone and one or more variable loops, in which the amino acid sequence of the loop or loops is specifically or randomly mutated to create an antigen-binding site having for binding the target antigen. Such proteins include the IgG-binding domains of protein A from S. aureus, transferrin, tetranectin, fibronectin (e.g. 10th fibronectin type III domain) and lipocalins. Other approaches include small constrained peptide e.g., based on “knottin” and cyclotides scaffolds [64]. Given their small size and complexity particularly in relation to correct formation of disulphide bond, there may be advantages to the use of eukaryotic cells for the selection of novel binders based on these scaffolds. Given the common functions of these peptides in nature, libraries of binders based on these scaffolds may be advantageous in generating small high affinity binders with particular application in blocking ion channels and proteases. In addition to antibody sequences and/or an antigen-binding site, a binder may comprise other amino acids, e.g., forming a peptide or polypeptide, such as a folded domain, or to impart to the molecule another functional characteristic in addition to ability to bind antigen. A binder may carry a detectable label, or may be conjugated to a toxin or a targeting moiety or enzyme (e.g., via a peptidyl bond or linker). For example, a binder may comprise a catalytic site (e.g., in an enzyme domain) as well as an antigen binding site, wherein the antigen binding site binds to the antigen and thus targets the catalytic site to the antigen. The catalytic site may inhibit biological function of the antigen, e.g., by cleavage.

### Antibody Molecules

Antibody molecules are preferred binders. Antibody molecules may be whole antibodies or immunoglobulins (Ig), which have four polypeptide chains-two identical heavy chains and two identical light chains. The heavy and light chains form pairs, each having a VH-VL domain pair that contains an antigen binding site. The heavy and light chains also comprise constant regions: light chain CL, and heavy chain CH1, CH2, CH3 and sometimes CH4 (the fifth domain CH4 is present in human IgM and IgE). The two heavy chains are joined by disulphide bridges at a flexible hinge region. An antibody molecule may comprise a VH and/or a VL domain.

The most common native format of an antibody molecule is an IgG which is a heterotetramer consisting of two identical heavy chains and two identical light chains. The heavy and light chains are made up of modular domains with a conserved secondary structure consisting of a four-stranded antiparallel beta-sheet and a three-stranded anti-parallel beta-sheet, stabilised by a single disulphide bond. Antibody heavy chains each have an N terminal variable domain (VH) and 3 relatively conserved “constant” immunoglobulin domains (CH1, CH2, CH3) while the light chains have one N terminal variable domain (VL) and one constant domains (CL). Disulphide bonds stabilise individual domains and form covalent linkages to join the four chains in a stable complex. The VL and CL of the light chain associates with VH and CH1 of the heavy chain and these elements can be expressed alone to form a Fab fragment. The CH2 and CH3 domains (also called the “Fc domain”) associate with another CH2: CH3 pair to give a tetrameric Y shaped molecule with the variable domains from the heavy and light chains at the tips of the “Y”. The CH2 and CH3 domains are responsible for the interactions with effector cells and complement components within the immune system. Recombinant antibodies have previously been expressed in IgG format or as Fabs (consisting of a dimer of VH: CH1 and a light chain). In addition the artificial construct called a single chain Fv (scFv) could be used consisting of DNA encoding VH and VL fragments fused genetically with DNA encoding a flexible linker.

Binders may be human antibody molecules. Thus, where constant domains are present these are preferably human constant domains.

Binders may be antibody fragments or smaller antibody molecule formats, such as single chain antibody molecules. For example, the antibody molecules may be scFv molecules, consisting of a VH domain and a VL domain joined by a linker peptide. In the scFv molecule, the VH and VL domains form a VH-VL pair in which the complementarity determining regions of the VH and VL come together to form an antigen binding site.

Other antibody fragments that comprise an antibody antigen-binding site include, but are not limited to, (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment [65, 66, 67], which consists of a VH or a VL domain; (v) isolated CDR regions; (vi) F(ab′)2 fragments, a bivalent fragment comprising two linked Fab fragments (vii) scFv, wherein a VH domain and a VL domain are linked by a peptide linker which allows the two domains to associate to form an antigen binding site [68, 69]; (viii) bispecific single chain Fv dimers (PCT/US92/09965) and (ix) “diabodies”, multivalent or multispecific fragments constructed by gene fusion (WO94/13804; [70]). Fv, scFv or diabody molecules may be stabilised by the incorporation of disulphide bridges linking the VH and VL domains [71].

Various other antibody molecules including one or more antibody antigen-binding sites have been engineered, including for example Fab2, Fab3, diabodies, triabodies, tetrabodies and minibodies (small immune proteins). Antibody molecules and methods for their construction and use have been described [72].

Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

A dAb (domain antibody) is a small monomeric antigen-binding fragment of an antibody, namely the variable region of an antibody heavy or light chain. VH dAbs occur naturally in camelids (e.g., camel, llama) and may be produced by immunizing a camelid with a target antigen, isolating antigen-specific B cells and directly cloning dAb genes from individual B cells. dAbs are also producible in cell culture. Their small size, good solubility and temperature stability makes them particularly physiologically useful and suitable for selection and affinity maturation. Camelid VH dAbs are being developed for therapeutic use under the name “nanobodies TM”.

Synthetic antibody molecules may be created by expression from genes generated by means of oligonucleotides synthesized and assembled within suitable expression vectors, for example as described by Knappik et al. or Krebs et al. [74].

Bispecific or bifunctional antibodies form a second generation of monoclonal antibodies in which two different variable regions are combined in the same molecule [75]. Their use has been demonstrated both in the diagnostic field and in the therapy field from their capacity to recruit new effector functions or to target several molecules on the surface of tumour cells. Where bispecific antibodies are to be used, these may be conventional bispecific antibodies, which can be manufactured in a variety of ways [76], e.g., prepared chemically or from hybrid hybridomas, or may be any of the bispecific antibody fragments mentioned above. These antibodies can be obtained by chemical methods [77, 78] or somatic methods [79, 80] but likewise and preferentially by genetic engineering techniques which allow the heterodimerisation to be forced and thus facilitate the process of purification of the antibody sought [81]. Examples of bispecific antibodies include those of the BiTETM technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction.

Bispecific antibodies can be constructed as entire IgG, as bispecific Fab′2, as Fab′PEG, as diabodies or else as bispecific scFv. Further, two bispecific antibodies can be linked using routine methods known in the art to form tetravalent antibodies.

Bispecific diabodies, as opposed to bispecific whole antibodies, may also be particularly useful. Diabodies (and many other polypeptides, such as antibody fragments) of appropriate binding specificities can be readily selected. If one arm of the diabody is to be kept constant, for instance, with a specificity directed against an antigen of interest, then a library can be made where the other arm is varied and an antibody of appropriate specificity selected. Bispecific whole antibodies may be made by alternative engineering methods as described in Ridgeway et al., 1996 [82].

A library according to the invention may be used to select an antibody molecule that binds one or more antigens of interest. Selection from libraries is described in detail below. Following selection, the antibody molecule may then be engineered into a different format and/or to contain additional features. For example, the selected antibody molecule may be converted to a different format, such as one of the antibody formats described above. The selected antibody molecules, and antibody molecules comprising the VH and/or VL CDRs of the selected antibody molecules, are an aspect of the present invention. Antibody molecules and their encoding nucleic acid may be provided in isolated form.

Antibody fragments can be obtained starting from an antibody molecule by methods such as digestion by enzymes e.g. pepsin or papain and/or by cleavage of the disulphide bridges by chemical reduction. In another manner, the antibody fragments can be obtained by techniques of genetic recombination well known to the person skilled in the art or else by peptide synthesis by means of, for example, automatic peptide synthesisers, or by nucleic acid synthesis and expression.

It is possible to take monoclonal and other antibodies and use techniques of recombinant DNA technology to produce other antibodies or chimaeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature.

Antibody molecules may be selected from a library and then modified, for example the in vivo half-life of an antibody molecule can be increased by chemical modification, for example PEGylation, or by incorporation in a liposome.

### Sources of Binder Genes

The traditional route for generation of monoclonal antibodies utilises the immune system of laboratory animals like mice and rabbits to generate a pool of high affinity antibodies which are then isolated by the use of hybridoma technology. The libraries of the present invention provides an alternative route to identifying antibodies arising from immunisation. VH and VL genes could be amplified from the B cells of immunised animals and cloned into an appropriate vector for introduction into eukaryotic libraries followed by selection from these libraries. Phage display and ribosome display allows very large libraries (>109 clones) to be constructed enabling isolation of human antibodies without immunisation. Producing libraries according to the present invention could also be used in conjunction with such methods. Following rounds of phage display selection, the selected population of binders could be introduced into eukaryotic cells by nuclease-directed integration as described herein. This would allow the initial use of very large libraries based in other systems (e.g., phage display) to enrich a population of binders while allowing their efficient screening using eukaryotic cells as described above. Thus the invention can combine the best features of both phage display and eukaryotic display to give a high throughput system with quantitative screening and sorting.

Using phage display and yeast display it has previously been demonstrated that it is also possible to generate binders without resorting to immunisation, provided display libraries of sufficient size are used. For example multiple binders were generated from a non-immune antibody library of >107 clones [83]. This in turn allows generation of binders to targets which are difficult by traditional immunisation routes e.g., generation of antibodies to “self-antigens” or epitopes which are conserved between species. For example, human/mouse cross-reactive binders can be enriched by sequential selection on human and then mouse versions of the same target. Since it is not possible to specifically immunise humans to most targets of interest, this facility is particularly important in allowing the generation of human antibodies which are preferred for therapeutic approaches.

In examples of mammalian display to date, where library sizes and quality were limited, binders have only been generated using repertoires which were pre-enriched for binders, e.g., from immunisation or from engineering of pre-existing binders. The ability to make large libraries in eukaryotic cells and particularly higher eukaryotes creates the possibility of isolating binders direct from these libraries starting with non-immune binders or binders which have not previously been selected within another system. By producing a library according to the present invention it is possible to generate binders from non-immune sources. This in turn opens up the possibilities for using binder genes from multiple sources. Binder genes could come from PCR of natural sources such as antibody genes. Binder genes could also be re-cloned from existing libraries, such as antibody phage display libraries, and cloned into a suitable donor vector for nuclease-directed integration into target cells. Binders may be completely or partially synthetic in origin. Furthermore various types of binders are described elsewhere herein, for example binder genes could encode antibodies or could encode alternative scaffolds [58, 59], peptides or engineered proteins or protein domains.

### Binder Display

A library built in the context of the invention may be cultured to express the binders in either soluble secreted form or in transmembrane form. It is said that the expressed binders are “displayed” if they are retained on the surface of the cells which encode them. In this context, terms like “binder display”, “display on/at the surface”, “display on the cell”, “display of the binder” and the like may be used interchangeably. In this context, a library may also be called a display or a display library.

Preferably, a library wherein the expressed binders are displayed is to provide a repertoire of binders for screening against a target of interest.

Binders may comprise or be linked to a membrane anchor, such as a transmembrane domain, for extracellular display of the binder at the cell surface. This may involve direct fusion of the binder to a membrane localisation signal such as a GPI recognition sequence or to a transmembrane domain such as the transmembrane domain of the PDGF receptor [84]. Retention of binders at the cell surface can also be done indirectly by association with another cell surface retained molecule expressed within the same cell. This associated molecule could itself be part of a heterodimeric binder, such as tethered antibody heavy chain in association with a light chain partner that is not directly tethered.

Although cell surface immobilisation facilitates selection of the binder, in many applications it is necessary to prepare cell-free, secreted binder. It will be possible to combine membrane tethering and soluble secretion using a recapture method of attaching the secreted binders to cell surface receptors. One approach is to format the library of binders as secreted molecules which can associate with a membrane anchored molecule expressed within the same cell which can function to capture a secreted binder. For example, in the case of antibodies or binder molecules fused to antibody Fc domains, a membrane tethered Fc can “sample” secreted binder molecules being expressed in the same cell resulting in display of a monomeric fraction of the binder molecules being expressed while the remainder is secreted in a bivalent form (U.S. Pat. No. 8,551,715). An alternative is to use a tethered IgG binding domain such as protein A.

Other methods for retaining secreted antibodies with the cells producing them are reviewed in Kumar et al. (2012) and include encapsulation of cells within microdrops, matrix aided capture, affinity capture surface display (ACSD), secretion and capture technology (SECANT) and “cold capture” [85]. In examples given for ACSD and SECANT [85], biotinylation is used to facilitate immobilisation of streptavidin or a capture antibody on the cell surface. The captured molecule in turn captures secreted antibodies. In the example of SECANT in vivo biotinylated of the secreted molecule occurs. Using the “cold capture” technique secreted antibody can be detected on producer cells using antibodies directed to the secreted molecule. It has been proposed that this due to association of the secreted antibody with the glycocalyx of the cell [86]. Alternatively it has been suggested that the secreted product is trapped by staining antibodies on the cell surface before being endocytosed [87]. The above methods have been used to identify high expressing clones within a population but could potentially be adapted for identification of binding specificity, provided the association has sufficient longevity at the cell surface.

Even when the binder is directly tethered to the cell surface it is possible to generate a soluble product. For example the gene encoding the selected binder can be recovered and cloned into an expression vector lacking the membrane anchored sequence. Alternatively, an expression construction can be used in which the transmembrane domain is encoded within an exon flanked by recombination sites, e.g., ROX recognition sites for Ore recombinase [88]. The exon encoding the transmembrane domain can be removed by transfection with a gene encoding Dre recombinase to switch expression to a secreted form.

Any of the above methods or other suitable approaches can be used to ensure that binders expressed by clones of a library are displayed on the surface of their expressing cells.

Display of scFvs on the Surface of Mammalian Cells Fused to Fc Domains

Although many antibody phage display libraries are formatted to display scFvs, eukaryotic display systems will allow presentation in Fab or IgG format. To take full advantage of the potential for IgG/Fab expression, particularly when using scFvs from other display systems will be necessary to take selected linked VH and VL domains within a bacterial expression system and express them within a eukaryotic system fused to appropriate constant domains. Described here is a method to convert scFv populations to immunoglobulin (Ig) or fragment, antigen binding (Fab) format in such a way that original VH and VL chain pairings are maintained. In the present invention, conversion is possible using individual clones, oligoclonal mixes or whole populations formatted as scFv while retaining the original pairing of VH and VLs chains. The method proceeds via the generation of an intermediate non-replicative “mini-circle” DNA which brings in a new “stuffer” DNA fragment. The circular DNA is linearised (e.g., by restriction digestion or PCR) which alters the relative position of the original VH and VL fragments and places the “stuffed” DNA between them. Following linearization the product can be cloned into a vector of choice, e.g., a mammalian expression vector. In this way all of the elements apart from the VH and VL can be replaced. Elements for bacterial expression can be replaced with elements for mammalian expression and fusion to alternative partners. The complete conversion process only requires a single transformation step of E. coli bacteria to generate a population of bacterial colonies each harbouring a plasmid encoding a unique Ig or Fab formatted recombinant antibody. Extending beyond conversion of scFv to IgG/Fab, the method can be employed to reformat any two joined DNA elements to clone into a vector such that after re-formatting each DNA element is surrounded by different DNA control features whilst maintaining the original pairing. A previous method has been described wherein 2 sequential cloning steps are used to replace these elements in contrast to the present method which proceeds via an intermediate non-replicative circular intermediate.

A method of restructuring a binder, or population of binders, may comprise converting scFv to Ig or a fragment thereof, e.g., Fab. The method may comprise converting nucleic acid encoding scFv to DNA encoding an immunoglobulin (Ig) or fragment thereof such as Fab format, in such a way that the original variable VH and VL chain pairings are maintained. Preferably the conversion proceeds via circular DNA intermediate which may be a non-replicative “mini-circle” DNA. The method requires a single transformation of E. coli for the direct generation of bacterial transformants harbouring plasmids encoding Ig or Fab DNA. The method may be used for monoclonal, oligoclonal or polyclonal clone reformatting. The method may be used to convert “en masse” an entire output population from any of the commonly used display technologies including phage, yeast or ribosome display.

More generally, this method allows the reformatting of any two joined DNA elements into a vector where the DNA elements are cloned under the control of separate promoters, or separated by alternative control elements, but maintaining the original DNA pairing.

Isolation, and optional restructuring, of DNA encoding binders may be followed by introduction of that DNA into further cells to create a derivative library as described elsewhere herein, or DNA encoding one or more particular binders of interest may be introduced into a host cell for expression. The host cell may be of a different type compared with the cells of the library from which it was obtained. Generally the DNA will be provided in a vector. DNA introduced into the host cell may integrate into cellular DNA of the host cell. Host cells expressing the secreted soluble antibody molecule can then be selected.

Host cells encoding one or more binders may be provided in culture medium and cultured to express the one or more binders.

### Derivative Libraries

Following a method for producing a library, one or more library clones may be selected and used to produce a further, second generation library. When a library has been generated by introducing DNA into eukaryotic cells as described herein, the library may be cultured to express the binders, and one or more clones expressing binders of interest may be recovered, for example by selecting binders against a target via a method for identifying a binder to a target. These clones may subsequently be used to generate a derivative library containing DNA encoding a second repertoire of binders, preferably via a method for producing a library.

To generate the derivative library, donor DNA of the one or more recovered clones is mutated to provide the second repertoire of binders. Mutations may be addition, substitution or deletion of one or more nucleotides. Where the binder is a polypeptide, mutation will be to change the sequence of the encoded binder by addition, substitution or deletion of one or more amino acids. Mutation may be focussed on one or more regions, such as one or more CDRs of an antibody molecule, providing a repertoire of binders of a common structural class which differ in one or more regions of diversity, as described elsewhere herein. Generating the derivative library may comprise isolating donor DNA from the one or more recovered clones, introducing mutation into the DNA to provide a derivative population of donor DNA molecules encoding a second repertoire of binders, and introducing the derivative population of donor DNA molecules into cells to create a derivative library of cells containing DNA encoding the second repertoire of binders.

Isolation of the donor DNA may involve obtaining and/or identifying the DNA from the clone. Such methods may encompass amplifying the DNA encoding a binder from a recovered clone, e.g., by PCR and introducing mutations. DNA may be sequenced and mutated DNA synthesised.

Mutation may alternatively be introduced into the donor DNA in the one or more recovered clones by inducing mutation of the DNA within the clones. The derivative library may thus be created from one or more clones without requiring isolation of the DNA, e.g., through endogenous mutation in avian DT40 cells. Antibody display lends itself especially well to the creation of derivative libraries. Once antibody genes are isolated, it is possible to use a variety of mutagenesis approaches (e.g., error prone PCR, oligonucleotide-directed mutagenesis, chain shuffling) to create display libraries of related clones from which improved variants can be selected. For example, with chain-shuffling the DNA encoding the population of selected VH clone, oligoclonal mix or population can be sub-cloned into a vector encoding a suitable antibody format and encoding a suitably formatted repertoire of VL chains [118]. Alternatively and again using the example of VHs, the VH clone, oligomix or population could be introduced into a population of eukaryotic cells which encode and express a population of appropriately formatted light chain partners (e.g., a VL-CL chain for association with an IgG or Fab formatted heavy chain). The VH population could arise from any of the sources discussed above including B cells of immunised animals or scFv genes from selected phage populations. In the latter example cloning of selected VHs into a repertoire of light chains could combine chain shuffling and re-formatting (e.g., into IgG format) in one step.

A particular advantage of display on eukaryotic cells is the ability to control the stringency of the selection/screening step. By reducing antigen concentration, cells expressing the highest affinity binders can be distinguished from lower affinity clones within the population. The visualisation and quantification of the affinity maturation process using flow cytometry is a major benefit of eukaryotic display as it gives an early indication of percentage positives in naive library and allows a direct comparison between the affinity of the selected clones and the parental population during sorting. Following sorting, the affinity of individual clones can be determined by pre-incubating with a range of antigen concentrations and analysis in flow cytometry or with a homogenous Time Resolved Fluorescence (TRF) assay or using surface plasmon resonance (SPR) (Biacore).

Screening to identify binders to a target of interestAs noted, the eukaryotic cell library may be used in a method of screening for a binder that recognises a target. Such a method may comprise:
providing a library via the method for producing a library of the invention, or providing a library via the use of a locus according to the invention, or providing a library according to the invention,
culturing cells of the library to express the binders,
exposing the binders to the target, allowing recognition of the target by one or more cognate binders, if present, and
detecting whether the target is recognised by a cognate binder.

A method according to this aspect may be called a method for identifying a binder to a target in the context of this application. In this context, the selection of binder or the screening for a binder also refer to such a method.

Methods for identifying a binder to a target may be carried out using a range of target molecule classes, e.g., protein, nucleic acid, carbohydrate, lipid, small molecules. The target may be provided in soluble form. The target may be labelled to facilitate detection, e.g., it may carry a fluorescent label or it may be biotinylated.

Cells expressing a target-specific binder may be isolated using a directly or indirectly labelled target molecule, where the binder captures the labelled molecule. For example, cells that are bound, via the binder: target interaction, to a fluorescently labelled target can be detected and sorted by flow cytometry or FACS to isolate the desired cells. Selections involving cytometry require target molecules which are directly fluorescently labelled or are labelled with molecules which can be detected with secondary reagents, e.g., biotinylated target can be added to cells and binding to the cell surface can be detected with fluorescently labelled streptavidin such as streptavidin-phycoerythrin. A further possibility is to immobilise the target molecule or secondary reagents which bind to the target on a solid surface, such as magnetic beads or agarose beads, to allow enrichment of cells which bind the target. For example cells that bind, via the binder: target interaction, to a biotinylated target can be isolated on a substrate coated with streptavidin, e.g., streptavidin-coated beads.

In libraries used in methods for identifying a binder to a target it is preferable to over-sample, i.e., screen more clones than the number of independent clones present within the library to ensure effective representation of the library. Identifying binders from very large libraries provided by the present invention could be done by flow sorting but this would take several days, particularly if over-sampling the library. As an alternative initial selections could be based on the use of recoverable antigen, e.g., biotinylated antigen recovered on streptavidin-coated magnetic beads. Thus streptavidin-coated magnetic beads could be used to capture cells which have bound to biotinylated antigen. Selection with magnetic beads could be used as the only selection method or this could be done in conjunction with flow cytometry where better resolution can be achieved, e.g., differentiating between a clone with higher expression levels and one with a higher affinity [56, 57].

The in vitro nature of display technology approaches makes it is possible to control selection in a way that is not possible by immunisation, e.g., selecting on a particular conformational state of a target [90, 91]. Targets could be tagged through chemical modification (fluorescein, biotin) or by genetic fusion (e.g. protein fused to an epitope tag such as a FLAG tag or another protein domain or a whole protein). The tag could be nucleic acid (e.g., DNA, RNA or non-biological nucleic acids) where the tag is part fused to target nucleic acid or could be chemically attached to another type of molecule such as a protein. This could be through chemical conjugation or through enzymatic attachment [92]. Nucleic acid could be also fused to a target through a translational process such as ribosome display. The “tag” may be another modification occurring within the cell (e.g., glycosylation, phosphorylation, ubiqitinylation, alkylation, PASylation, SUMO-lation and others described at the Post-translational Database (db-PTM) at http://dbptm.mbc.nctu.edu.tw/statistics.php) which can be detected via secondary reagents. This would yield binders which bind an unknown target protein on the basis of a particular modification.

Targets could be detected using existing binders which bind to that target molecule, e.g., target specific antibodies. Use of existing binders for detection will have the added advantage of identifying binders within the library of binders which recognise an epitope distinct from the binder used for detection. In this way pairs of binders could be identified for use in applications such as sandwich ELISA. Where possible a purified target molecule would be preferred. Alternatively the target may be displayed on the surface of a population of target cells and the binders are displayed on the surface of the library cells, the method comprising exposing the binders to the target by bringing the library cells into contact with the target cells. Recovery of the cells expressing the target (e.g., using biotinylated cells expressing target) will allow enrichment of cells which express binders to them. This approach would be useful where low affinity interactions are involved since there is the potential for a strong avidity effect.

The target molecule could also be unpurified recombinant or unpurified native targets provided a detection molecule is available to identify cell binding (as described above). In addition binding of target molecules to the cell expressing the binder could be detected indirectly through the association of target molecule to another molecule which is being detected, e.g., a cell lysate containing a tagged molecule could be incubated with a library of binders to identify binders not only to the tagged molecule but also binders to its associated partner proteins. This would result in a panel of antibodies to these partners which could be used to detect or identify the partner (e.g., using mass spectrometry). Cellular fractionation could be used to enrich targets from particular sub-cellular locations. Alternatively differential biotinylation of surface or cytoplasmic fractions could be used in conjunction with streptavidin detection reagents for eukaryotic display [93, 94]. The use of detergent solubilised target preparations is a particularly useful approach for intact membrane proteins such as GPCRs and ion channels which are otherwise difficult to prepare. The presence of detergents may have a detrimental effect on the eukaryotic cells displaying the binders requiring recovery of binder genes without additional growth of the selected cells.

Following detection of target recognition by a cognate binder, cells of a clone containing DNA encoding the cognate binder may be recovered. DNA encoding the binder may then be isolated (e.g., identified or amplified) from the recovered clone, thereby obtaining DNA encoding a binder that recognises the target.

Exemplary binders and targets are detailed elsewhere herein. A classic example is a library of antibody molecules, which may be screened for binding to a target antigen of interest. Other examples include screening a library of TCRs against a target MHC: peptide complex or screening a library of MHC: peptide complexes against a target TCR.

### TCR: MHC and Other Receptor Interactions

As explained above, the binder and the target in a method for identifying a binder to a target may be a TCR and a MHC: peptide complex, respectively, et vice versa. Hence, display libraries may be libraries of TCRs on surface of yeast cells and mammalian cells. Such libraries may be used to select TCRs with altered recognition properties. Alternatively, display libraries may be libraries of peptides or MHC variants for recognition by TCRs.

T cell receptors (TCRs) are expressed on T cells and have evolved to recognise peptide presented in complex with MHC molecules on antigen presenting cells. TCRs are heterodimers consisting in 95% of cases of alpha and beta heterodimers and in 5% of cases of gamma and delta heterodimers. Both monomer units have an N terminal immunoglobulin domain which has 3 variable complementarity determining regions (CDRs) involved in driving interaction with target. The functional TCR is present within a complex of other sub-units and signalling is enhanced by co-stimulation with CD4 and CD8 molecules (specific for class | and class II MHC molecules respectively). On antigen presenting cells, proteins are processed, and presented on the cell surface in complex with MHC molecules which are themselves part of a multimeric protein complex. TCRs recognizing peptides originating from “self” are removed during development and the system is poised for recognition of foreign peptides presented on antigen presenting cells to effect an immune response. The outcome of recognition of a peptide: MHC complex depends on the identity of the T cell and the affinity of that interaction.

It is valuable to identify the genes encoding TCRs or MHC: peptide complexes which drive interactions involved in pathological conditions, e.g., as occurs in autoimmune disease. It would be desirable to engineer TCRs for altered binding e.g. higher affinity to targets of interest, e.g., in re-targeting T cells in cancer or enhancing the effect of existing T cells [95]. Alternatively the behaviour of regulatory or suppressive T cells might be altered as a therapeutic modality, e.g., for directing or enhancing immunotherapy of cancer by introducing specific TCRs into T cells or by using expressed TCR protein as therapeutic entities [96].

Display of libraries of TCRs on surface of yeast cells and mammalian cells has previously been demonstrated. In the case of yeast cells it was necessary to engineer the TCR and present it in a single chain format. Since the affinity of interaction between TCR and peptide: MHC complex is low, the soluble component (e.g., peptide: MHC in this case) is usually presented in a multimeric format. TCR specificity has been engineered for peptides in complex with MHC class | and MHC class II [98]. TCRs have also been expressed on the surface of a mutant mouse T cells (lacking TCR alpha and beta chains) and variant TCRs with improved binding properties have been isolated [99]. For example Chervin et al. introduced TCRs by retroviral infection and an effective library size of 104 clones was generated [100]. Using nuclease-directed integration of binders as proposed here, a similar approach could be taken to engineering T cells. As well as selecting TCRs with altered recognition properties, display libraries could be used to screen libraries of peptide or of MHC variants for recognition by TCRs. For example peptide: MHC complexes have been displayed on insect cells and used to epitope map TCRs presented in a multimeric format [101]. As noted, screening methods may involve displaying the repertoire of binders on the cell surface and probing with a target presented as a soluble molecule, which may be a multimeric target. An alternative, which can be especially useful with multimeric targets, is to screen directly for cell: cell interactions, where binder and target are presented on the surface of different cells. For example if activation of a TCR of interest led to expression of a reporter gene this could be used to identify activating peptides or activating MHC molecules presented within a peptide: MHC library. In this particular example the reporter cell does not encode the library member but could be used to identify the cell which does encode it. The approach could potentially extend to a “library versus library” approach. For example extending the example described above, a TCR library could be screened against a peptide: MHC library. More broadly the example of screening a library of binders presented on one cell surface using a binding partner on another cell could be extended to other types of cell: cell interactions e.g., identification of binders which inhibit or activate signalling within the Notch or Wnt pathways. Thus the present invention could be used in alternative cell based screening system including recognition systems based on cell: cell interactions.

As an example, chimeric antigen receptors (“CARS”) represent a fusion between an antibody binding domain (usually formatted as a scFv) and a signalling domain. These have been introduced into T cells with the aim of re-directing the T cell in vivo to attack tumour cells through antibody recognition and binding to tumour-specific antigens. A number of different factors could affect the success of this strategy including the combination of antibody specificity, format, antibody affinity, linker length, fused signalling module, expression level in T cells, T cell sub-type and interaction of the CAR with other signalling molecules [102, 103]. The ability to create large libraries of CARs in primary T cells incorporating individual or combinations of the above variables would allow a functional search for effective and optimal CAR construction. This functional “search” could be carried out in vitro or in vivo. For example Alonso-Camino (2009) have fused a scFv recognizing CEA to the chain of the TCR: CD3 complex and introduced this genetic construct into a human Jurkat cell line [104]. Upon interaction with CEA present on either Hela cells or tumour cells they showed upregulation of the early T cell activation marker CD69. This approach could be used to identify CAR fusion constructs with appropriate activation or inhibitory properties using cultured or primary cells. Further functionality of CAR constructs may be assessed in vivo. For example a library of CARs constructed in primary mouse T cells may be introduced into tumour bearing mice to identify T cell clones stimulated to proliferate through encounter with tumour. If necessary this T cell library could be pre-selected based on antigen binding specificity using the methods described above. In either case the incoming library of binders could be used to replace an existing binder molecule (e.g., MHC or TCR or antibody variable domain).

### Phenotype Screens

In a preferred method for identifying a binder to a target the binder is ably to modify cell signaling and/or cellular behavior as a result of the action the binder on the target. In more preferred methods, the binder is an antibody.

Antibodies which modify cell signalling by binding to ligands or receptors have a proven track record in drug development and the demand for such therapeutic antibodies continues to grow. Such antibodies and other classes of functional binders also have potential in controlling cell behaviour in vivo and in vitro. The ability to control and direct cellular behaviour however relies on the availability of natural ligands which control specific signalling pathways. Unfortunately many natural ligands such as those controlling stem cell differentiation (e.g., members of FGF, TGF-beta, Wnt and Notch super-families) often exhibit promiscuous interactions and have limited availability due to their poor expression/stability profiles. Due to their specificity, antibodies have great potential in controlling cellular behaviour.

The identification of functional antibodies that modify cell signalling has historically been relatively laborious involving picking clones, expressing antibody, characterising according to sequence and binding properties, conversion to mammalian expression systems and addition to functional cell based assays. The eukaryotic display approach described herein will reduce this effort but there is still a requirement for production of antibody and addition to a separate reporter cell culture. Therefore, a preferred alternative may be to directly screen libraries of binders expressed in eukaryotic cells for the effect of binding on cell signalling or cell behaviour by using the production cell itself as a reporter cell. Following introduction of antibody genes, clones within the resulting population of cells showing alteration in reporter gene expression or altered phenotypes can be identified.

A number of recent publications have described the construction of antibody libraries by cloning repertoires of antibody genes into reporter cells [47, 105, 106]. These systems combine expression and reporting within one cell, and typically introduce a population of antibodies selected against a pre-defined target (e.g., using phage display).

A population of antibody genes may be introduced into reporter cells to produce a library by methods described herein, and clones within the population with an antibody-directed alteration in phenotype (e.g., altered gene expression or survival) can be identified. For this phenotypic-directed selection to work there is a requirement to retain a linkage between the antibody gene present within the expressing cell (genotype) and the consequence of antibody expression (phenotype). This has been achieved previously either through tethering the antibody to the cell surface as described for antibody display or through the use of semi-solid medium to retain secreted antibodies in the vicinity of producing cells [105]. Alternatively antibodies and other binders can be retained inside the cell [107]. Binders retained on the cell surface or in the surrounding medium can interact with an endogenous or exogenous receptor on the cell surface causing activation of the receptor. This in turn can cause a change in expression of a reporter gene or a change in the phenotype of the cell. As an alternative the antibody can block the receptor or ligand to reduce receptor activation. The gene encoding the binder which causes the modified cellular behaviour can then be recovered for production or further engineering.

An alternative to this “target-directed” approach, it is possible to introduce a “naive” antibody population which has not been pre-selected to a particular target [108]. The cellular reporting system is used to identify members of the population with altered behaviour. Since there is no prior knowledge of the target, this non-targeted approach has a particular requirement for a large antibody repertoire, since pre-enrichment of the antibody population to the target is not possible. This approach will benefit from using nuclease-directed transgene integration as described in the present invention.

The “functional selection” approach could be used on other applications involving libraries in eukaryotic cells, particularly higher eukaryotes such as mammalian cells. The antibody could be fused to a signalling domain such that binding to target causes activation of the receptor. Kawahara et al. have constructed chimeric receptors where an extra-cellular scFv targeting fluorescein was fused to a spacer domain (the D2 domain of the Epo receptor) and various intracellular cytokine receptor domain including the thrombopoeitin (Tpo) receptor, erythropoietin (Epo) receptor, gp130, IL-2 receptor and the EGF receptor [109, 110, 111]. These were introduced into an IL-3 dependent proB cell line (BaF3) [27], where chimaeric receptors were shown to exhibit antigen-dependent activation of the chimaeric receptor leading to IL-3 independent growth. This same approach was used in model experiments to demonstrate antigen mediated chemoattraction of BaF3 cells [110]. The approach was extended beyond stable culture cells to primary cells exemplified by the survival and growth of Tpo-responsive haematopoeitic stem cells or IL2 dependant primary T cells where normal stimulation by Tpo and IL-2 respectively was replaced by fluorescein directed stimulation of scFv chimaeric receptors. Thus a system based on chimaeric antibody-receptor chimaeras can be used to drive target dependent gene expression or phenotypic changes in primary or stable reporter cells. This capacity could be used to identify fused binders which drive a signalling response or binders which inhibit the response.

In a modification of the above approach separate VH and VL domains from an anti-lysozyme antibody were fused to the Epo intracellular domain [113]. Cells grew in response to addition of lysozyme indicating an antigen induced dimerisation or stabilisation of the separate VH and VL fusion partners. Thus three interacting components come together for an optimal response in this system. Although described here with reference to antibody molecules, the above methods for identifying a binder (i.e. an antibody molecule) to a target may also be adapted and performed with libraries of other types of binders.

Protein fragment complementation represents an alternative system for studying and for selecting protein: protein interactions in mammalian cells [114, 115]. This involves restoring function of split reporter proteins through protein: protein interactions. Reporter proteins which have been used include ubiquitin, DNAE intein, beta-galactosidase, dihydrofolate reductase, GFP, firefly luciferase, beta-lactamase, TEV protease. For example a recent example of this approach is the mammalian membrane 2 hybrid (MaMTH) approach where association of a bait protein: split ubiquitin: transcription factor fusion with a partner protein: split ubiquitin restores ubiquitin recognition and liberates the transcription factor to effect reporter gene expression [116]. Again binders which interfere with or enhance this interaction could be identified through perturbed signalling.

### Recovery and Reformatting of Binders and Encoding DNA

After a binder is identified via a method for identifying a binder to a target, a common next step will be to isolate (e.g., identify or amplify) the DNA encoding the binder. Optionally, it may be desired to modify the nucleic acid encoding the binder, for example to restructure the binder and/or to insert the encoding sequence into a different vector. Hence, a preferred method for identifying a binder to a target comprises isolating the DNA encoding the binder recognizing the target. More preferred methods are described below. Where the binder is an antibody molecule, a preferred method for identifying a binder to a target comprises isolating DNA encoding the antibody molecule from cells of a clone, amplifying DNA encoding at least one antibody variable region, preferably both the VH and VL domain, and inserting DNA into a vector to provide a vector encoding the antibody molecule. A multimeric antibody molecule bearing a constant domain may be converted to a single chain antibody molecule for expression in a soluble secreted form.

Antibodies may be presented in different formats but whatever format an antibody is selected in, once the antibody gene is isolated it is possible to reconfigure it in a number of different formats. Once VH or VL domains are isolated, they can be re-cloned into expression vectors encompassing the required partner domains.

A more preferred method for identifying a binder to a target comprises a reformatting step comprising the reformatting of binders composed of a pair of subunits (e.g., scFv molecules), to a different molecular binder format (e.g., Ig or Fab) in which the original pairing of the subunits is maintained. Such methods are described in more detail elsewhere herein and can be used for monoclonal, oligoclonal or polyclonal clone reformatting. The method can be used to convert “en masse” an entire output population from any of the commonly used display technologies including phage, yeast or ribosome display.

Additional embodiments of the invention are set out in the following numbered paragraphs which form part of the description.

1. A method for identifying a locus in a genome of a eukaryotic cell, said locus being a candidate for insertion of binder sequences, said method comprising:
a. providing a landing pad sequence;
b. introducing the landing pad sequence into the eukaryotic cell;
c. randomly integrating the landing pad sequence into the genome of the eukaryotic cell via transposon-mediated integration;
d. selecting a clone having a landing pad sequence integrated into its genome.

2. The method of paragraph 1 comprising the further steps of:
e. screening for single-copy integration;
f. identifying the locus.

3. The method of paragraph 2 comprising the additional steps of:
g. integrating a donor DNA sequence comprising one or more transgenes encoding a binder at the landing pad sequence;
h. screening for integration of the donor DNA.

4. The method of any one of paragraphs 1-3, wherein the landing pad sequence comprises a recognition sequence for a site-specific nuclease.

5. The method of paragraph 4, wherein the nuclease recognition sequence is a meganuclease recognition sequence, a zinc finger nuclease recognition sequence, a TALE nuclease recognition sequence or a nucleic acid guided nuclease recognition sequence, preferably a meganuclease recognition sequence.

6. The method of paragraph 5, wherein the nuclease recognition sequence is a I-SceI meganuclease recognition sequence.

7. The method of any one of paragraphs 4-6, wherein step g of integrating the donor DNA into the cells comprises providing a site-specific nuclease within the cells, wherein the nuclease cleaves the recognition sequence comprised in the landing pad.

8. The method of any one of paragraphs 3-7, wherein step h of screening for integration of the donor DNA comprises screening for display of the one or more binders encoded by the donor DNA.

9. The method of any one of paragraphs 3-8, wherein the donor DNA further comprises homology arms to increase integration efficiency.

10. The method of any one of paragraphs 1-9, wherein the landing pad sequence and/or the donor DNA sequence comprise a selectable marker.

11. Use of the locus identified in the method of any one of paragraphs 1 through 10 for building a library of eukaryotic cell clones containing DNA encoding a diverse repertoire of binders.

12. An in vitro library of eukaryotic cell clones that express a diverse repertoire of at least 10\hat{ }3, 10\hat{ }4, 10\hat{ }5, 10\hat{ }6, 10\hat{ }7, 10\hat{ }8 or 10\hat{ }9 different binders, each cell containing recombinant DNA wherein donor DNA encoding a binder or subunit of a binder is integrated in a fixed locus in the cellular DNA, the locus being identified by a method according to any one of paragraphs 1-10.

13. An in vitro library of eukaryotic cell clones according to paragraph 12, wherein donor DNA encoding a binder or subunit of a binder is integrated in at least a first and/or a second fixed locus in the cellular DNA, wherein said fixed locus or loci are identified by a method according to any one of paragraphs 1-10.

14. An in vitro library of eukaryotic cell clones according to paragraph 12 or 13, wherein the locus or loci are in a gene selected from an NLN gene, a TNIK gene, a PARP11 gene, a RAB40B gene, an ABI2 gene, an RNF19B gene, a PKIA gene, or an FTCD gene.

15. An in vitro library of eukaryotic cell clones according to any of paragraphs 12-14, wherein the locus or loci are in an NLN gene, a TNIK gene or a RAB40B gene, preferably in an NLN gene.

16. An in vitro library of eukaryotic cell clones according to any of paragraphs 12-15, wherein the locus or loci are in an intron of the gene.

17. An in vitro library of eukaryotic cell clones according to any of paragraphs 12-16, wherein the locus or loci are in an open chromatin region of the intron.

18. An in vitro library of eukaryotic cell clones according to any of paragraphs 12-17, wherein the locus or loci are in an enhancer region of the intron.

19. An in vitro library of eukaryotic cell clones according to any of paragraphs 12-18, wherein the locus or loci are in NLN-207 intron 1, 2 or 6 of the NLN gene.

20. A binder identified from a library according to any of paragraphs 12-19.

21. A method for producing a library of eukaryotic cell clones containing DNA encoding a diverse repertoire of binders, comprising:
providing donor DNA molecules encoding the binders, and eukaryotic cells;
introducing the donor DNA into the cells and providing a site-specific nuclease within the cells, wherein the nuclease cleaves a recognition sequence in cellular DNA, wherein the recognition sequence is in an NLN gene, a TNIK gene, a PARP11 gene, a RAB40B gene, an AB/2 gene, an RNF19B gene, a PKIA gene, or an FTCD gene, to create an integration site at which the donor DNA becomes integrated into the cellular DNA, integration occurring through DNA repair mechanisms endogenous to the cells, thereby creating recombinant cells containing donor DNA integrated in the cellular DNA; and
culturing the recombinant cells to produce clones, thereby providing a library of eukaryotic cell clones containing donor DNA encoding the repertoire of binders.

22. A method according to paragraph 21, wherein the recognition sequence is in an NLN gene, a TNIK gene or a RAB40B gene.

23. A method according to paragraph 21 or 22, wherein the recognition sequence is in an NLN gene.

24. A method according to any one of paragraphs 21-23, wherein the recognition sequence is in an intron of the gene.

25. A method according to paragraph 24, wherein the recognition sequence is in an open chromatin region of the intron.

26. A method according to paragraph 24 or 25, wherein the recognition sequence is in an enhancer region of the intron.

27. A method according to any one of paragraphs 24-26, wherein the recognition sequence is in NLN-207 intron 1, 2 or 6 of the NLN gene.

28. A method according to any one of paragraphs 21-27, wherein the binders are antibody molecules.

29. A method according to paragraph 28, wherein the antibody molecules are full length immunoglobulins, IgG, Fab, scFv-Fc or scFv.

30. A method according to any of paragraphs 21-29, wherein the binders are multimeric, comprising at least a first and a second subunit.

31. A method of producing a library of eukaryotic cell clones containing DNA encoding a diverse repertoire of multimeric binders, each binder comprising a first and a second subunit, wherein the method comprises
providing eukaryotic cells containing DNA encoding the first subunit and providing donor DNA molecules encoding the second binder subunit,
introducing the donor DNA into the cells and providing a site-specific nuclease within the cells, wherein the nuclease cleaves a recognition sequence in cellular DNA as defined in any of paragraphs 12-18 to create an integration site at which the donor DNA becomes integrated into the cellular DNA, integration occurring through DNA repair mechanisms endogenous to the cells, thereby creating recombinant cells which contain donor DNA integrated in the cellular DNA, and
culturing the recombinant cells to produce clones containing DNA encoding the first and second subunits of the multimeric binder.

32. A method of producing a library of eukaryotic cell clones containing DNA encoding a diverse repertoire of multimeric binders, each binder comprising at least a first and a second subunit, wherein the method comprises
providing first donor DNA molecules encoding the first subunit, and providing eukaryotic cells
introducing the first donor DNA into the cells and providing a site-specific nuclease within the cells, wherein the nuclease cleaves a recognition sequence in cellular DNA as defined in any of paragraphs 12-18 to create an integration site at which the donor DNA becomes integrated into the cellular DNA, integration occurring through DNA repair mechanisms endogenous to the cells, thereby creating a first set of recombinant cells containing first donor DNA integrated in the cellular DNA,
culturing the first set of recombinant cells to produce a first set of clones containing DNA encoding the first subunit,
introducing second donor DNA molecules encoding the second subunit into cells of the first set of clones, wherein the second donor DNA is integrated into cellular DNA of the first set of clones, thereby creating a second set of recombinant cells containing first and second donor DNA integrated into the cellular DNA, and
culturing the second set of recombinant cells to produce a second set of clones, these clones containing DNA encoding the first and second subunits of the multimeric binder,
thereby providing a library of eukaryotic cell clones containing donor DNA encoding the repertoire of multimeric binders.

33. A method according to paragraph 32, wherein the second donor DNA molecules are integrated by a method comprising providing a site-specific nuclease within the cells, wherein the nuclease cleaves a recognition sequence in cellular DNA to create an integration site at which the donor DNA becomes integrated into the cellular DNA, integration occurring through DNA repair mechanisms endogenous to the cells.

34. A method according to any of paragraphs 21-33, wherein the repertoire of binders is a plurality of polypeptides which share a common structure and have one or more regions of amino acid sequence diversity.

35. A method according to any of paragraphs 21-34, wherein the repertoire of binders is a repertoire of antibody molecules differing in one or more complementarity determining regions.

36. A method according to any of paragraphs 29-35 wherein the multimeric binders are antibody molecules comprising a heavy chain variable (VH) domain and a light chain variable (VL) domain as separate subunits.

37. A method according to paragraph 36, wherein the multimeric binders are whole immunoglobulins.

38. A method according 36, wherein the multimeric binders are IgG.

39. A method according to paragraph 36, wherein the multimeric binders are Fab.

40. A method according to any of paragraphs 36 to 39, wherein the first subunit comprises the VH domain and wherein the second subunit comprises the VL domain.

41. A method according to any of paragraphs 36 to 39, wherein the first subunit comprises the VL domain and wherein the second subunit comprises the VH domain.

42. A method according to any of paragraphs 36 to 41 wherein the antibody molecule further comprises one or more additional subunits, which may be introduced on the same donor DNA as the first or second subunit or which may be integrated at separate sites in the cellular DNA.

43. A method according to any of paragraphs 21-42, wherein the cells are higher eukaryotic cells with a genome size of greater than 2×10\hat{ }7 base pairs.

44. A method according to any of paragraphs 21-43, wherein the cells are mammalian, avian, insect or plant cells.

45. A method according to paragraph 44, wherein the cells are mammalian, preferably wherein the cells are human.

46. A method according to paragraph 45, wherein the cells are HEK293 cells, Chinese hamster ovary (CHO) cells, T lymphocyte lineage cells or B lymphocyte lineage cells or any of the cell lines listed in the “Cancer Cell Line Encyclopedia” or “COSMIC catalogue of somatic mutations in cancer”.

47. A method according to paragraph 46, wherein the cells are primary T cells or a T cell line.

48. A method according to paragraph 46, wherein the cells are primary B cells, a B cell line, a pre-B cell line or a pro-B cell line.

49. A method according to paragraph 48, wherein the cells are murine pre-B cell line 1624-5, IL-3 dependent pro-B cell line Ba/F3 or chicken DT40 B cells.

50. A method according to any of paragraphs 21-49, wherein the recognition sequence for the site-specific nuclease occurs only once or twice in the cellular DNA.

51. A method according to any of paragraphs 21-50, wherein the site-specific nuclease cleaves cellular DNA to create a double strand break serving as an integration site.

52. A method according to any of paragraphs 21-51, wherein the nuclease is a meganuclease.

53. A method according to any of paragraphs 21-51, wherein the nuclease is a zinc finger nuclease (ZFN).

54. A method according to any of paragraphs 21-51, wherein the nuclease is a TALE nuclease.

55. A method according to any of paragraphs 21-51, wherein the nuclease is a nucleic acid guided nuclease.

56. A method according to paragraph 55, wherein DNA cleavage is directed by the CRISPR/Cas system.

57. A method according to any of paragraphs 21-56, wherein the donor DNA is integrated into the cellular DNA by homologous recombination.

58. A method according to any of paragraphs 21-56, wherein the donor DNA is integrated into the genomic DNA by non-homologous end joining or microhomology-directed end joining.

59. A method according to any of paragraphs 21-58, wherein the donor DNA comprises a genetic element for selection of cells into which the donor DNA is integrated.

60. A method according to any of paragraphs 21-59, wherein integration of the donor DNA into the cellular DNA places expression of the binder and/or expression of a genetic selection element under control of a promoter present within the cellular DNA.

61. A method according to any of paragraphs 21-58, wherein the donor DNA comprises a sequence encoding the binder operably linked to a promoter.

62. A method according to any of paragraphs 21-61, wherein the library contains at least 100, 10\hat{ }3, 10\hat{ }4, 10\hat{ }5 or 10\hat{ }6 clones, each clone being derived from an individual recombinant cell produced by integration of donor DNA.

63. A method according to any of paragraphs 21-62, wherein the library encodes at least 100, 10\hat{ }3, 10\hat{ }4, 10\hat{ }5 or 10\hat{ }6 different binders.

64. A method according to any of paragraphs 21-63, wherein each clone contains integrated donor DNA encoding only one or two members of the repertoire of binders.

65. A method according to any of paragraphs 21-64, wherein the eukaryotic cells are diploid and contain a recognition sequence for the site-specific nuclease at duplicate fixed loci in the cellular DNA.

66. A method according to any of paragraphs 21 to 63, wherein each clone contains integrated donor DNA encoding a single member of the repertoire of binders.

67. A method according to any of paragraphs 21-66, wherein the donor DNA molecules each encode a single binder or binder subunit.

68. A method according to any of paragraphs 21-67, wherein the binders are displayed on the cell surface.

69. A method according to any of the paragraphs 21-68, wherein the binders are secreted from the cells.

70. A method according to any of paragraphs 21-69, further comprising:
culturing the library to express the binders,
recovering one or more clones expressing a binder of interest, and
generating a derivative library from the one or more recovered clones, wherein the derivative library contains DNA encoding a second repertoire of binders.

71. A method according to paragraph 70, wherein generating the derivative library comprises isolating donor DNA from the one or more recovered clones, introducing mutation into the DNA to provide a derivative population of donor DNA molecules encoding a second repertoire of binders, and introducing the derivative population of donor DNA molecules into cells to create a derivative library of cells containing DNA encoding the second repertoire of binders.

72. A method according to paragraph 70, wherein generating the derivative library comprises introducing mutation into the donor DNA in the one or more recovered clones by inducing mutation of the DNA within the clones.

73. A method of producing a diverse repertoire of binders, comprising producing a library by a method according to any of paragraphs 21-72 and culturing the library cells to express the binders.

74. A library produced by a method according to any of paragraphs 21 to 72.

75. A method of screening for a cell of a desired phenotype, wherein the phenotype results from expression of a binder by the cell, the method comprising
providing a library via the method for producing a library according to any one of paragraphs 21 to 72, or providing a library via the use of paragraph 11, or providing a library according to any of claims 12-19 or 74,
culturing the library cells to express the binders, and
detecting whether the desired phenotype is exhibited.

76. A method according to paragraph 75, wherein the phenotype is expression of a reporter gene in a cell that expresses the binder.

77. A method according to paragraph 75 or paragraph 76, further comprising recovering cells of a clone that expresses a binder that produces the desired phenotype.

78. A method according to paragraph 77, further comprising isolating DNA encoding the binder from the recovered clone, thereby obtaining DNA encoding a binder which produces the desired phenotype.

79. A method for screening to identify a binder to a target of interest, said method comprising:
providing a library via the method for producing a library according to any one of paragraphs 21 to 72, or providing a library via the use of paragraph 11, or providing a library according to any of claims 12-19 or 74,
culturing cells of the library to express the binders,
exposing the binders to the target, allowing recognition of the target by one or more cognate binders, if present, and
detecting whether the target is recognised by a cognate binder. 80. A method according to paragraph 75 or 79, wherein the target is provided in soluble form.

81. A method according to paragraph 75 or paragraph 79, wherein the target is displayed on the surface of a population of target cells and the binders are displayed on the surface of the library cells, the method comprising exposing the binders to the target by bringing the library cells into contact with the target cells.

82. A method according to any of paragraphs 75 to 81, wherein the binders are antibody molecules and the target is an antigen.

83. A method according to any of paragraphs 75 to 81, wherein the binders are TCRs and the target is an MHC: peptide complex.

84. A method according to any of paragraphs 79 to 83, further comprising detecting target recognition by a cognate binder, and recovering cells of a clone containing DNA encoding the cognate binder.

85. A method according to paragraph 84, further comprising isolating DNA encoding the binder from the recovered clone, thereby obtaining DNA encoding a binder that recognises the target.

86. A method according to paragraph 78 or paragraph 85, comprising introducing mutation or converting the DNA to modified DNA encoding a restructured binder.

87. A method according to paragraph 86, wherein the binder is a scFv and the method comprises converting DNA encoding the scFv to DNA encoding an Ig or fragment thereof while maintaining the original variable VH and VL chain pairings.

88. A method according to paragraph 78, 85, 86 or 87, further comprising introducing the DNA into a host cell.

### General Information

Unless stated otherwise, all technical and scientific terms used herein have the same meaning as customarily and ordinarily understood by a person of ordinary skill in the art to which this invention belongs, and read in view of this disclosure.

### Sequence Identity

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively, percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Schematic representation of the plnt105 vector comprising a transposon (TR) flanked I-scel landing pad cassette. The landing pad includes a promoter (mPGK) driving expression of a short first exon (Ex1) followed by an intronic sequence containing the I-SceI meganuclease recognition sequence. Annotations: TR—inverted terminal repeats of PiggyBac transposon; mPGK—mouse phosphoglycerate kinase promoter; Ex1—exon 1 of mouse phosphoglycerate kinase; LHA—left homology arm; I-SceI-meganuclease cleavage site; RHA—right homology arm; Ubi—ubiquitin promoter; Puro—puromycin gene; LoxP—locus of cross-over (LoxP and Lox2272 are CRE recombinase sites).

FIG. 2A-2B. Dot plots showing Fc expression 6 days post transfection without blasticidin selection. HEK293F cells transfected with pINT17-bococizumab and pINT17-5A10i either in the presence or absence of AAVS TALEN nuclease are shown in FIG. 2A. HEK293F cells transfected with pINT74-bococizumab and pINT74-5A10i either in the presence or absence of or NLN CRISPR are shown in FIG. 2B.

FIG. 3A-3B. Histogram overlay plots showing Fc expression 15 days post transfection (BSD resistant population) (FIG. 3A) and 28 days post transfection (FIG. 3B) in cells transfected with pINT17-bococizumab and 5A10i in the presence of AAVS TALEN nuclease and with pINT74-bococizumab and pINT74-5A10i in the presence of NLN CRISPR. Arrows denote the histogram plot of AAVS integrations.

FIG. 4A-4B. NLN CHO-K1 gene structure is shown in FIG. 4A, with exons indicated by numbered boxes. FIG. 4B shows the GC content of NLN CHO Intron 1 first 68 kb.

FIG. 5A-5B. FIG. 5A shows the nuclease cleavage position in NLN CHO Intron 1. FIG. 5B shows the TALEN nuclease right arm DNA insert for CHO NLN intron 1 integration.

FIG. 6. Antibody expression profiles 2 days post transfection, measured using flow-cytometry based analysis by staining with an anti-human Fc antibody. Left: 884_01_G01 integrated at the AAVS locus; Middle: 5A10i integrated at NLN intron 1; Right: bococizumab integrated at NLN intron 1. PcDNA stands for transfections without nuclease.

FIG. 7A-7B. Antibody expression profiles 8 days post transfection (FIG. 7A) and 14 days post transfection (FIG. 7B) in cells resistant to BSD, measured using flow-cytometry based analysis by staining with an anti-human Fc antibody. Left: 884_01_G01 integrated at the AAVS locus; Middle: 5A10i integrated at NLN intron 1; Right: bococizumab integrated at NLN intron 1. Shown percentages refer to anti-human Fc antibody-stained cell population whereas shown numbers refer to cell counts. PcDNA stands for transfections without nuclease.

FIG. 8. Antibody expression profiles 1 day post transfection (left), 7 days post transfection (middle), and 14 days post transfection (right) in cells resistant to BSD, measured using flow-cytometry based analysis by staining with an anti-human Fc antibody. 5A10i and bococizumab were integrated in NLN intron 1 (pINT58-5A10i and pINT58-bococizumab). PcDNA stands for transfections without nuclease.

FIG. 9. Integration efficiency (%) measured using flow-cytometry based analysis by staining with an anti-human Fc antibody 7 days (left) and 14 days (middle) post transfection without blasticidin selection. 5A10i and bococizumab were integrated in NLN intron 1 (pINT58-5A10i and pINT58-bococizumab). On the right integration efficiency without the use of nuclease is shown.

### OVERVIEW OF SEQUENCES

| SEQ | |
|---|---|
| ID NO: | Description of the sequence |
| 1 | NLN gene |
| 2 | TNIK gene |
| 3 | PARP 11 gene |
| 4 | RAB40B gene |
| 5 | ABI2 gene |
| 6 | RNF19B gene |
| 7 | PKIA gene |
| 8 | FTCD gene |
| 9 | NLN-201 intron 1 |
| 10 | NLN-201 intron 2 |
| 11 | NLN-201 intron 3 |
| 12 | NLN-201 intron 4 |
| 13 | NLN-201 intron 5 |
| 14 | NLN-201 intron 6 |
| 15 | NLN-201 intron 7 |
| 16 | NLN-201 intron 8 |
| 17 | NLN-201 intron 9 |
| 18 | NLN-201 intron 10 |
| 19 | NLN-201 intron 11 |
| 20 | NLN-201 intron 12 |
| 21 | NLN-207 intron 5 |
| 22 | NLN-207 intron 6 |
| 24 | 3′SPLNK-PB-SEQ |
| 25 | 5′SPLNK-PB-SEQ |
| 26 | SPLNK-GATC-TOP |
| 27 | SPLNK-BOT |
| 28 | SPLNK#1 |
| 29 | SPLNK#2 |
| 30 | 3′SPLNK-PB#1 |
| 31 | 3′SPLNK-PB#2 |
| 32 | 5′SPLNK-PB#1 |
| 33 | 5′SPLNK-PB#2 |
| 34 | Clone A02-Chr. 7 |
| 35 | Clone A04-Chr. 5 |
| 36 | Clone A05-Chr. 3 |
| 37 | Clone A06-Chr. 12 |
| 38 | Clone A07-Chr. 17 |
| 39 | Clone A08-multiple hits |
| 40 | Clone A09-Chr. 7 |
| 41 | Clone A10-Chr. 11 |
| 42 | Clone B04-Chr. 2 |
| 43 | Clone B06-Chr. 3 |
| 44 | Clone B07-Chr. 1 |
| 45 | Clone B11-Chr. 8 |
| 46 | Clone B12- Chr. 15 |
| 47 | Clone C01-Chr. 17 |
| 48 | Clone C02-Chr. 2 |
| 49 | Clone C03-Chr. 9 |
| 50 | gRNA targeting intron 2 |
| | of the neurolysin gene |
| 51 | LHA-Seq_Forw primer |
| 52 | LHA_Seq rev primer |
| 53 | RHA_Seq_Forw primer |
| 54 | RHA_Seq_Rev primer |
| 55 | BSD-Seq-Forw primer |
| 56 | BSD-BstBI-Rev primer |
| 57 | NLN Intron 2-CRISPR-1-gBlock |
| 58 | NLN Intron 2-LHA and RHA-gBlock |
| 59 | pINT105 vector |
| 60 | LHA_AsiSI-Forw |
| 61 | NLN_LHA_Nsil-Rev |
| 62 | NLN_RHA_BstZ171_Forw |
| 63 | RHA_Sbfl-Rev |
| 64 | pINT74-5A10i |
| 65 | pINT74-bococizumab |
| 66 | CHO NLN-intron 1 TALEN |
| | recognition site |
| 67 | pINT157-884_01_G01 |
| 68 | pINT158-bococizumab |
| 69 | pINT158-5A10i |
| 70 | I-Scel recognition sequence |
| 71 | nuclease cleavage position in |
| | NLN CHO Intron 1 |
| 72 | over-lap |
| 73 | CHO NLN intron 1 integration site |
| 74 | 3356 |
| 75 | 3357 |
| 76 | meganuclease family |
| | characteristic sequence 1 |
| 77 | meganuclease family |
| | characteristic sequence 2 |

### EXAMPLES

### Example 1: Generation of Hek293 Cell Lines with Integrated I-SceI Meganuclease Recognition Site Via Transposon-Mediated Integration

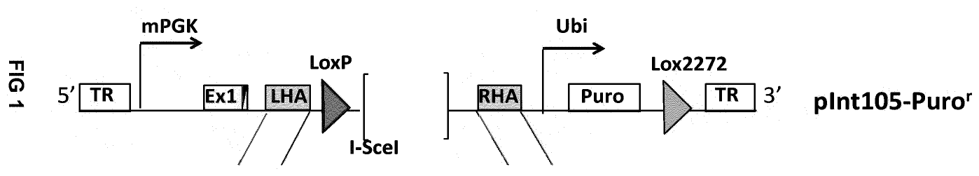

1×10⁷ Hek293 cells were co-transfected with a pINT105 vector comprising a Piggybac (PB) transposon terminal repeat (TR) flanking a landing pad comprising a recognition sequence for I-SceI meganuclease and a puromycin resistance gene driven by the ubiquitin promoter (FIG. 1; SEQ ID NO: 59), a pcDNA 3.0 vector, and a PBase vector (encoding mPB transposase) using Maxcyte (MD, USA) electroporation following the manufacturer's protocol, using an OC100 cuvette. The pcDNA 3.0 vector is an empty vector used as a carrier to normalize the DNA concentration in transfections. Control transfections without the PBase vector were also performed. The DNA amounts used are given in Table 1. After 24 h, transfected cells were plated in 10% DMEM agar plates. 24 h post plating, 2 μg/ml puromycin was added to the cells. Two weeks after puromycin selection, 48 colonies were picked and expanded. Cell pellets were frozen down for genomic DNA extraction. One vial of 10⁷ cells per clone was frozen down and stored in liquid nitrogen.

TABLE 1
DNA amounts used in Hek293 transfection

| | Piggybac vector | | | |
|---|---|---|---|---|
| Reaction | (I-Scel-recognition | | | |
| No | site-landing pad) | PB vector | pcDNA3.0 | Total DNA |
| 1 | 30 ng | 250 ng | 19.72 μg | 20 μg |
| 2 | 30 ng | — | 19.97 μg | 20 μg |
| 3 | 150 ng | 250 ng | 19.6 μg | 20 μg |
| 4 | 150 ng | — | 19.85 μg | 20 μg |

20 clones were mapped for the genomic location of landing pad integration using splinkerette PCR as described by Potter and Luo (2010) PLOS ONE 5 (4): e1016. 1 μg of genomic DNA per clone was digested with Sau3a. Then, splinkerette PCR was performed as follows:

### Step 1: Reaction Conditions for Annealing Splinkerette Oligonucleotides

### Component Volume

SPLNK-BOT (150 ng/μl) 50 μl
SPLNK-GATC-TOP (150 ng/μl) 50 μl
10×NEB Buffer 2 100 μl
H₂O 800 μl
Total 1000 μl
Heated to 95° C. for 3 minutes. Allowed to cool on bench to room temp (˜30 mins). Stored 200 μl aliquots at −20° C.

### Step 2: Ligation to Splinkerette Oligonucleotide Conditions for Ligating Digested Genomic DNA to Annealed Splinkerette Oligonucleotide

### Component Volume

Digested genomic DNA 35 μl
H₂O 2.5 μl
10×NEB Ligase Buffer 5 μl
annealed splinkerette oligonucleotide (from step 1) 6 μl
NEB T4 DNA Ligase (400 U/μl) 1.5 μl
Total 50 μl
Incubated at room temperature ≥2 hrs. Proceeded directly to Round 1 PCR.

### Step 3. Round 1 Splinkerette PCR

### Round 1 PCR Reaction.

### Components Volume

Ligated genomic DNA 10 μl
H₂O 8.25 μl
5× Phusion HF Buffer 5 μl
10 mm dNTP 0.5 μl
SPLNK #1, 10 μM 0.5 μl
Primer PB #1, 10 μM (see Table 2) 0.5 μl
Phusion Taq (Finnzymes, FL) 0.25 μl
Total 25 μl
   PCR conditions for round 1 PCR.
98° C. 75 sec
98° C. 20 sec→64° C. 15 sec (×2 cycles)
98° C. 20 sec→58 or 64° C. 15 sec (58° C. for 3′SPLNK-PB #1 and 64° C. for 5′SPLNK-PB #1, Table 2)→
72° C. 2 min (×30 cycles)
72° C. 7 min
4° C. hold

### Step 4. Round 2 Splinkerette PCR

### Round 2 PCR Reaction.

### Component Volume

Round 1 PCR product 1 μl (diluted 2×)
H₂O 31.5 μl
5× Phusion HF Buffer 10 μl
10 mm dNTP 1 μl
SPLNK #2, 10 μM 1 μl
Primer PB #2, 10 μM (see Table 2) 1 μl
Phusion Taq 0.5 μl
Total 50 μl

### PCR Conditions for Round 2 PCR.

98° C. 75 sec
98° C. 20 sec→59 or 66° C. 15 sec (59° C. for 3′SPLNK-PB #2 and 66° C. for 5′SPLNK-PB #2, Table 2)—+72° C. 90 sec (×30 cycles)
72° C. 7 min
4° C. hold

### Step 5. Antarctic Phosphatase/Exonuclease I Treatment AntPho/ExoI Reaction Conditions.

### Components Volume

Round 2 splinkerette PCR product 20 μl
10×NEB AP Buffer 3.0 μl
H₂O 3.0 μl
NEB Antarctic Phosphatase 2.0 μl (New England Biolabs, MA, USA)
NEB Exonuclease I 2.0 μl (New England Biolabs, MA, USA)
Total 30 μl

Incubated reactions at 37° C. for 2 hrs, followed by a 80° C. incubation for 15 min. Used 15 μl of the reaction for sequencing with the appropriate sequencing primer (SEQ ID NO: 17, SEQ ID NO: 18, Table 2).

TABLE 2
Primer sequences used

| | SEQ | | |
|---|---|---|---|
| | ID | | |
| Purpose | NO | Name | Sequence 5′ → 3′ |
| Sequencing | 24 | 3′SPLNK-PB- | ACGCATGATTATCTTTAAC |
| | | SEQ | |
| Sequencing | 25 | 5′SPLNK-PB- | CGACTGAGATGTCCTAAATGC |
| | | SEQ | |
| PCR | 26 | SPLNK-GATC- | GATCCCACTAGTGTCGACACC |
| | | TOP | AGTCTCTAATTTTTTTTTTCA |
| | | | AAAAAA |
| PCR | 27 | SPLNK-BOT | CGAAGAGTAACCGTTGCTAGG |
| | | | AGAGACCGTGGCTGAATGAGA |
| | | | CTGGTGTCGACACTAGTGG |
| PCR | 28 | SPLNK#1 | CGAAGAGTAACCGTTGCTAGG |
| | | | AGAGACC |
| PCR | 29 | SPLNK#2 | GTGGCTGAATGAGACTGGTGT |
| | | | CGAC |
| PCR | 30 | 3′SPLNK-PB#1 | GTTTGTTGAATTTATTATTAG |
| | | | TATGTAAG |
| PCR | 31 | 3′SPLNK-PB#2 | CGATAAAACACATGCGTC |
| PCR | 32 | 5′SPLNK-PB#1 | ACCGCATTGACAAGCACG |
| PCR | 33 | 5′SPLNK-PB#2 | CTCCAAGCGGCGACTGAG |

PCR was performed for 5′ end of the transposable element, checked on 0.8% agarose gel and sequenced. Obtained sequences were blasted using NCBI-blastn and the genomic locus was identified for each cell line (Table 3):

TABLE 3
Clones and identified loci with integrated transposons comprising the
landing pad sequence with the I-Scel recognition site

| | | | | | Additional |
|---|---|---|---|---|---|
| | | | | | features |
| | Chromosome | | | | (describing |
| | with | | | | gene regulatory |
| | integrated | | | | elements like |
| | transposon | | | | enhancers and |
| | comprising | | | | open chromatin, |
| | the landing | Locus | Ensemble | Intron | as indicated by |
| Clone | pad | (gene | Transcript | location | the Ensemble |
| name | sequence | name) | ID | (size) | genome browser) |
| A02 | 7 | No specific gene | | — | |
| | | identified | | | |
| A04 | 5 | neurolysin | NLN-207 | Intron6-7 | |
| | | | ENST00000509935.2 | (10,589 bp) | |
| A05 | 3 | TRAF2 and NCK | TNIK-204 | Intron2-3 | has open chromatin |
| | | interacting kinase | ENST00000436636.7 | (141,398 bp) | in the intron |
| | | (TNIK) | | | |
| A06 | 12 | poly-ADP- | PARP11-205 | Intron1-2 | |
| | | ribosyltransferase | ENST00000450737.2 | (9254 bp) | |
| | | 11 (PARP11) | | | |
| A07 | 17 | RAB40B | RAB40B-206 | Intron1-2 | has open chromatin |
| | | | ENST00000571995.6 | (33,898 bp) | and enhancers |
| A08 | multiple hits | — | | — | |
| A09 | 7 | No specific gene | | — | |
| | | identified | | | |
| A10 | 11 | No specific gene | In close proximity | — | |
| | | identified | to HSD17B12 | | |
| A11 | read not | — | | — | |
| | determinable | | | | |
| B01 | no hit | — | | — | |
| B04 | 2 | ABI2 | ABI2-203 | Intron1-2 | |
| | | | ENST00000261018.12 | (38,245 bp) | |
| B06 | 3 | TRAF2 and | TNIK-204 | Intron2-3 | has open chromatin |
| | | NCK interacting | ENST00000436636.7 | (141,398 bp) | in the intron |
| B07 | 1 | RNF19B | RNF19B-201 | Intron1-2 | |
| | | | ENST00000235150.5 | (14,276 bp) | |
| B09 | no hit | — | | — | |
| B10 | no hit | — | | — | |
| B11 | 8 | PKIA | PIKA-202 | Intron1-2 | Has 2 open |
| | | | ENST00000396418.7 | (56,342 bp) | chromatin sites |
| B12 | 15 | No specific gene | | — | has open chromatin |
| | | identified | | | |
| C01 | 17 | RAB40B | RAB40B-206 | Intron1-2 | has open chromatin |
| | | | ENST00000571995.6 | (33,898 bp) | and enhancers |
| C02 | 2 | FTCD | FTCDNL1-201 | Intron3-4 | has open chromatin |
| | | | ENST00000416668.5 | (85,239 bp) | and enhancers |
| C03 | 9 | No specific gene | | — | |
| | | identified | | | |

The sequencing results per clone are as follows: Clone A02-Chr. 7 (SEQ ID NO: 34), Clone A04-Chr. 5 (SEQ ID NO: 35), Clone A05-chr. 3 (SEQ ID NO: 36), Clone A06-Chr. 12 (SEQ ID NO: 37), Clone A07-Chr. 17 (SEQ ID NO: 38), Clone A08-multiple hits (SEQ ID NO: 39), Clone A09-Chr. 7 (SEQ ID NO: 40), Clone A10-Chr. 11 (SEQ ID NO: 41), Clone B04-Chr. 2 (SEQ ID NO: 42), Clone B06-Chr. 3 (SEQ ID NO: 43), Clone B07-Chr. 1 (SEQ ID NO: 44), Clone B11-Chr. 8 (SEQ ID NO: 45), Clone B12- Chr. 15 (SEQ ID NO: 46), Clone C01-Chr. 17 (SEQ ID NO: 47), Clone C02-Chr. 2 (SEQ ID NO: 48), Clone C03-Chr. 9 (SEQ ID NO: 49).

### Example 2: Validation of the Cell Lines by Transfecting with SceI Meganuclease and FGFR1 and FGFR2 as Donor DNA

The clones generated by PB transposon-mediated integration were validated for integration efficiency, single-copy integration analysis and antibody expression. For this purpose, the clones were co-transfected with equal proportion of two donor plasmids containing anti-FGFR1 scFv and anti-FGFR2 scFv in Fc format in the presence or absence of I-SceI meganuclease plasmid. Transfection with a mixture of anti-FGFR1 and a-FGFR2 antibodies affords an opportunity to examine the proportion of cells containing multiple integration events. For an individual cell with a correctly integrated cassette (e.g., anti-FGFR1) there is approximately a 50:50 chance that a second integration will be of the alternative specificity (i.e., anti-FGFR2). If there are frequent multiple integrations, then the proportion of double-positive clones will be high. The donor plasmids were described in WO2015/166272.

Transfection was performed in two batches: Batch 1 included clones A09, A10, B03, B05, B07, B09 and B12. Batch 2 included A02, A04, A05, A06, A07, A08, A11, B01, B04, B06, B10, B11, C01, C02 and C03. The day prior to transfection, the cells or HEK293-F cells were seeded at 0.5×10⁶ cells/ml. On the day of transfection, the cells were transfected using Maxcyte (MD, USA) electroporation following the manufacturer's protocol. Donor DNA (anti-FGFR1 and anti-FGFR2) was 1 μg for each in all transfections (including controls). Meganuclease DNA was 20 μg. HEK293F TALEN cells were used as controls in both batches (described in WO2015/166272). Control cells were transfected with equal proportion of two donor plasmids containing anti-FGFR1 scFv and anti-FGFR2 scFv in Fc format in the presence or absence of AAVS TALE nuclease plasmid which can integrate binders in the AAVS locus via TALEN-mediated integration. The TALEN DNA used was 10 μg (TAL L) and 10 μg (TAL R).

Transfected cells were plated in 10 cm petri dishes and were subjected to blasticidin selection after 2 days of transfection. Media in the plates were replenished with fresh media containing blasticidin every 3-4 days until day 20. Blasticidin-resistant colonies were scored to calculate the integration efficiency. Integration efficiency ranged from 0-1% and the fold-difference between the plus and minus nuclease varied among the clones. Clones A09, B12, and C03 showed higher fold difference between the plus and minus I-SceI meganuclease compared to other clones. Among these three clones, C03 showed the highest integration efficiency (1%). Looking at the integration efficiency of the remaining clones, A04 and B01 had 0.5% and 0.6% respectively.

In parallel, the transfected cells were also cultured in suspension and subjected to BSD selection from day 2 until day 20 with media change every 3-4 days. At day 20, cells were dual-stained with FGFR1-Dy633 and

FGFR2-Dy488. Flow cytometry-based analysis was performed using an Intellicyt IQUE screener (Sartorius AG, GE). The cytometer was equipped with 488 (blue) and 640 nm (red) lasers and emission filter for PE (LP: —, BP: 572/28) and To-Pro3 (LP: —, BP: 675/30). For the FGR1/2 staining, 10 nM of FGFR1-Dy633 and FGFR2-Dy488 were added to 100 μl of cells. These were incubated for 30 minutes in the dark at 4° C. 900 μl of 0.1% PBS was then added before centrifugation at 600×g for 2.5 minutes. The cells were washed with 1 ml of 0.1% BSA, and resuspended in 500 μl of 0.1% BSA. 50 μl was removed and added to wells of a 96 well plate for subsequent analysis. Dual staining of a cell with the antigens can indicates multiple-copy integration (more than one antibody gene integrated per cell). Antibody-negative populations were observed in all transfected clones. These populations were removed from the calculations of single-copy and multiple-copy integration analysis. The results are given in Table 4:

TABLE 4
% of FGR1-stained, FGR2-stained, and double FGR1-/FGR2-stained cells
(double positive), calculated with negative population removed.
HEK293F-TALEN cells integrate binders in the AAVS locus.

| Clone | FGR1-stained | FGR2-stained | Double positive |
|---|---|---|---|
| A09 | 49 | 41 | 10 |
| A10 | 44 | 48 | 9 |
| B07 | 46 | 47 | 8 |
| B09 | 46 | 47 | 6 |
| B12 | 42 | 51 | 5 |
| HEK293F TALEN | 39 | 50 | 11 |
| A02 | 55 | 41 | 4 |
| A04 | 36 | 57 | 7 |
| A05 | 45 | 46 | 9 |
| A06 | 43 | 53 | 4 |
| A07 | 42 | 50 | 7 |
| A08 | 52 | 38 | 10 |
| A11 | 48 | 48 | 4 |
| B01 | 42 | 48 | 10 |
| B04 | 46 | 49 | 5 |
| B06 | 55 | 41 | 4 |
| B10 | 44 | 47 | 9 |
| B11 | 61 | 33 | 7 |
| C01 | 41 | 51 | 8 |
| C02 | 46 | 43 | 9 |
| C03 | 39 | 53 | 8 |
| HEK293F TALEN | 48 | 39 | 13 |

### Example 3: Evaluation of the Neurolysin (NLN) Gene for Expression of Binders

To evaluate the capacity of the NLN locus for binder expression, a design for genomic integration of a cassette comprising a promoterless blasticidin gene and a gene expressing the anti-PCSK9 antibodies 5A10i or bococizumab (Boco) in NLN was made. Vectors pINT17-5A10i and pINT17-bococizumab originate from the pINT17-BSD vector (described in Parthiban et al. 2019 mAbs, 11:5, 884-898) which is a second-generation display vector which is a derivative of the pD2 vector described in WO2015/166272, which directs integration of comprised genes in the AAVS locus, in which cassettes comprising 5A10i and bococizumab expressing genes were inserted, respectively.

Integration of the cassette was achieved via CRISPR-mediated integration via the inclusion of a gRNA targeting intron 2 of the neurolysin gene (NLN-207) (TCACTCGTATTACGTTTACA, SEQ ID NO: 50) in the cassette. Homology arms of 800 bp were included in the cassette at either end to facilitate homologous recombination. The left homology arm (LHA) was flanked by an AsiSI recognition site on the 5′end and an NsiI recognition site on the 3′ end. The right homology arm (RHA) was flanked by a BstZ171 recognition site on the 5′end and a SbfI recognition site on the 3′ end. The left homology arm was amplified by PCR using primers LHA_AsiSI-Forw (SEQ ID NO: 60) and NLN_LHA-NsiI-Rev (SEQ ID NO: 61) and right homology arm was amplified using primers NLN-RHA-BstZ171_Forw (SEQ ID NO: 62) and RHA_SbfI-Rev (SEQ ID NO: 63) (primers are shown in Table 5) and ligated to the cassettes followed by restriction-digestion of the PCR products and vectors pINT17-5A10i and pINT17-bococizumab using the abovementioned enzymes and ligation. This resulted in pINT74-5A10i (SEQ ID NO: 64) and pINT74-bococizumab (SEQ ID NO: 65) vectors, which direct integration of the comprised genes in intron 2 of NLN (NLN-207).

TABLE 5
Primers

| SEQ | | |
|---|---|---|
| ID | | |
| NO | Primer | Sequence |
| 51 | LHA-Seq_Forw | TGCCAGATTCAGCAACGGAT |
| 52 | LHA_Seq_rev | GTCTTCGGAGATGGGGATGC |
| 53 | RHA_Seq_Forw | AGTTTTTCCTGCACGGGTAG |
| | | T |
| 54 | RHA_Seq_Rev | GCTTAATGCGCCGCTACAG |
| 55 | BSD-Seq-Forw | ACCTGTATCGTGGCCATTGG |
| 56 | BSD-BstBI-Rev | CGCTTGGTCGGTCATTTCG |
| 60 | LHA_AsiSI-Forw | CTTTGTTTTTTCGCGATCGC |
| 61 | NLN_LHA_Nsil-Rev | AAAACCTTGATGCATAACTC |
| | | ACGTC |
| 62 | NLN_RHA_BstZ171_Forw | ATTGGGTTTAAGTATACGGG |
| | | TAGGTA |
| 63 | RHA_Sbfl-Rev | TTCAGGAAAAACACCTGCAG |
| | | G |

Template sequences used: NLN Intron 2-CRISPR-1-gBlock (SEQ ID NO: 57), NLN Intron 2-LHA and RHA-gBlock (SEQ ID NO: 58).

Correct integration of the cassette in the targeted locus results in the expression of the blasticidin resistance gene and allows for correctly integrated clones to grow in the presence of the antibiotic, as described in WO2015/166272. The integration efficiency of the cassettes designed for integration in NLN intron 2 (NLN-207) was compared with the integration efficiency of cassettes designed for integration in the AAVS locus via TALEN-mediated integration, as described in WO2015/166272. HEK293F cells were transfected using Maxcyte electroporation (MD, USA), following the manufacturer's protocol. Control transfections without the TALEN- or CRISPR vectors were also performed. Table 6 shows the transfection conditions:

TABLE 6
Maxcyte transfection of HEK293F cells.
Total amount of cells used was 1 × 10⁷.
Total amount of DNA used was 22 μg.
Total reaction volume was 0.1 ml.
Cell concentration and DNA concentration
values were 1 × 10⁸ cells/ml and 220 μg/ml, respectively.
Amount of pINT17-Boco, pINT74-Boco,
pINT17-5A10i, pINT74-5A10i used was 2 μg. pcDNA3.0 (20 μg)
was used as a control for the samples without the TALE nucleases
or CRISPR/CAS9.

| | | AAVS TALEN | | |
|---|---|---|---|---|
| Reaction | Donor DNA | (left and right) | NLN-CRISPR | pcDNA 3.0 |
| 1 | pINT17-Boco | 10 + 10 μg | — | — |
| 2 | pINT17-Boco | — | — | 20 μg |
| 3 | pINT17-5A10i | 10 + 10 μg | — | |
| 4 | pINT17-5A10i | — | — | 20 μg |
| 5 | pINT74-Boco | — | 20 μg | — |
| 6 | pINT74-Boco | — | — | 20 μg |
| 7 | pINT74-5A10i | — | 20 μg | |
| 8 | pINT74-5A10i | — | — | 20 μg |

Following transfection, cells were plated in the presence or absence of blasticidin. Blasticidin resistant colonies were stained with methylene blue 12 days post-transfection, and counted to measure integration efficiency. Integration efficiency for cells transfected with AAVS TALEN was 0.26% for pINT17-Boco and 0.35% for pINT17-5A10i and with NLN CRISPR was 0.23% for pINT74-bococizumab and 0.53% for pINT74-5A10i (Table 7).

TABLE 7
BSD resistant colonies stained with methylene blue.
10⁴ cells were originally plated per plate.

| | | BSD resistant | Integration | |
|---|---|---|---|---|
| Reaction | Donor DNA | colonies | efficiency | Condition |
| 1 | pINT17-Boco | 26 | 0.26 | plus AAVS TALEN |
| 2 | pINT7-Boco | 1 | 0.01 | minus nuclease |
| 3 | pINT17-5A10i | 35 | 0.35 | plus AAVS TALEN |
| 4 | pINT17-5A10i | 0 | 0 | minus nuclease |
| 5 | pINT74-Boco | 23 | 0.23 | plus CRISPR |
| 6 | pINT74-Boco | 3 | 0.03 | minus CRISPR |
| 7 | pINT74-5A10i | 53 | 0.53 | plus CRISPR |
| 8 | pINT74-5A10i | 9 | 0.09 | minus CRISPR |

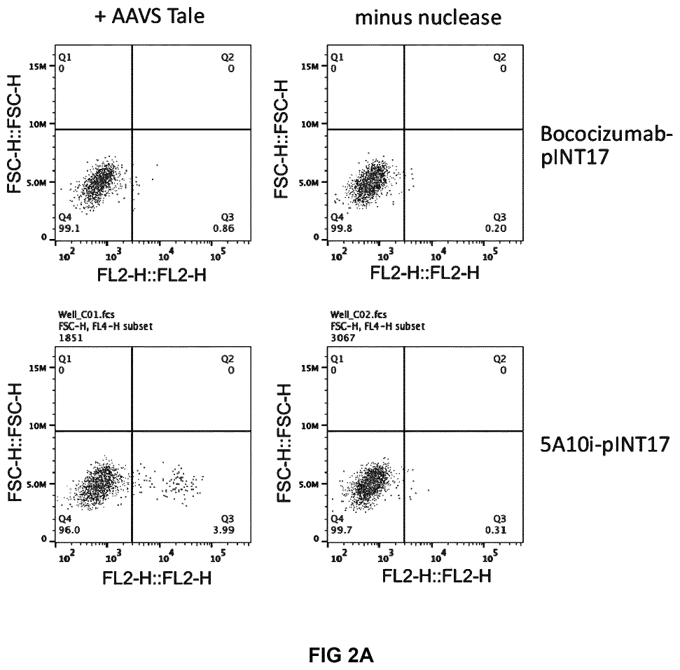

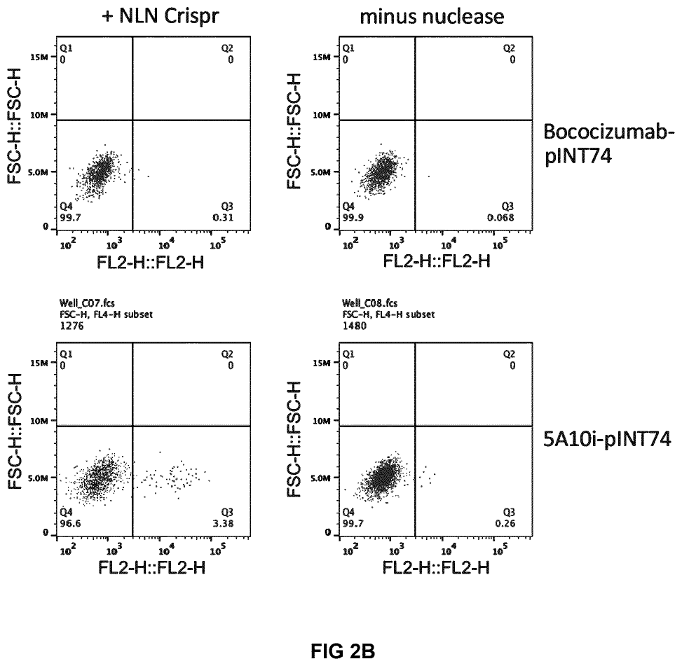

Integration efficiency was also quantitated by measuring the Fc expression at 6 days post transfection in the cells without BSD selection using flow cytometry-based analysis by staining with an anti-human Fc antibody (BioLegend cat #409304). Flow cytometry-based analysis was performed using an Intellicyt IQUE screener (Sartorius AG, GE). The cytometer was equipped with 488 (blue) and 640 nm (red) lasers and emission filter for PE (LP: —, BP: 572/28) and To-Pro3 (LP: —, BP: 675/30). For anti-human Fc antibody staining, 1×10⁶ cells were washed 2× with 0.1% BSA PBS, incubated with 1 μl of the labelling antibody in 100 μl of 1% BSA PBS (7.5% BSA Fraction V-Gibco (Cat. No: 15260037) for 30 minutes at 4° C., protected from light, washed again with PBS and analysed. The difference in the number of Fc positive cells between the presence and absence of nuclease is a way to measure the integration efficiency. Cells transfected with 5A10i in the presence of nuclease show 3.99% and 3.38% for AAVS TALEN- and NLN CRISPR-respectively (FIGS. 2A and 2B).

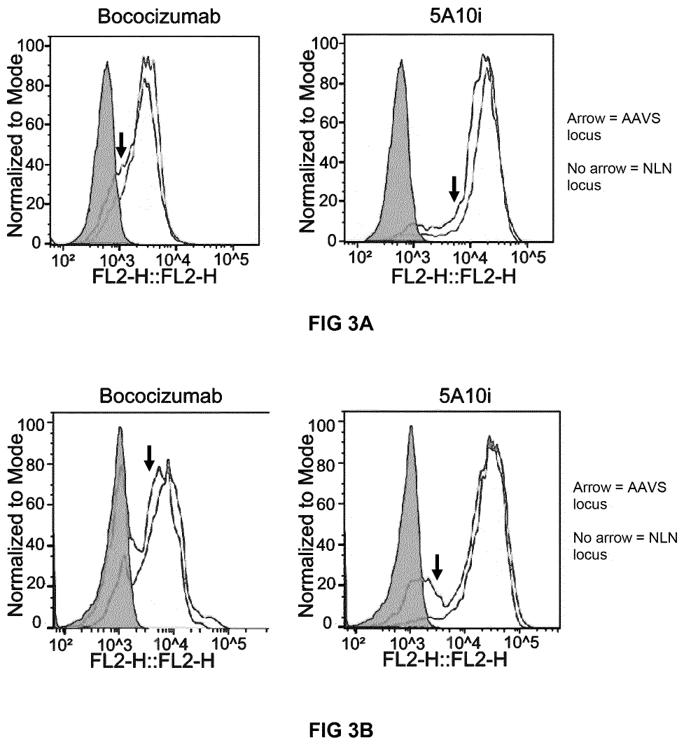

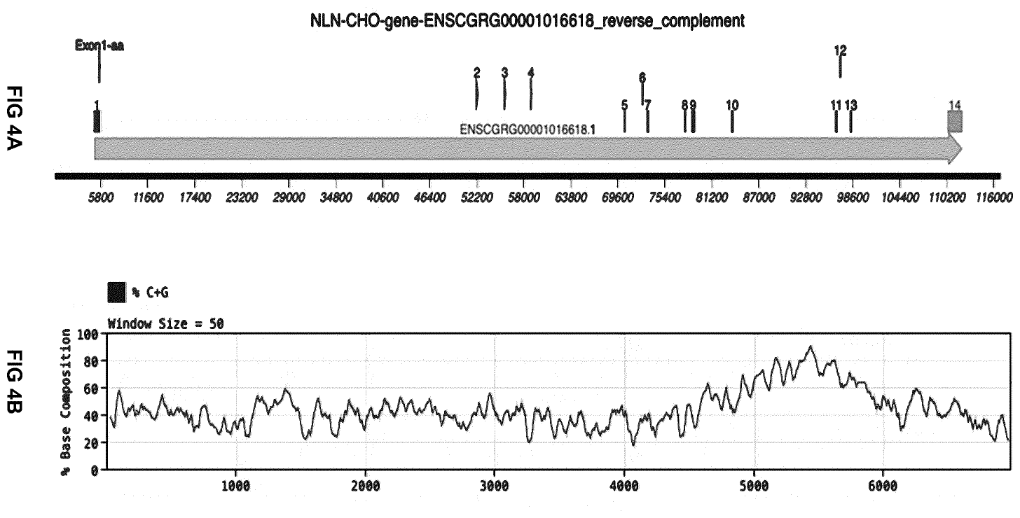

In parallel the transfected cells were also cultured in suspension and subjected to BSD selection. 15 days post-transfection, cells were stained with the anti-human Fc antibody and subjected to flow-cytometry based analysis. The BSD-resistant population showed Fc expression on the cell surface and the expression level looks similar between the cells integrated in AAVS and NLN loci (FIG. 3A):

Further, 28 days post-transfection, the stability of Fc expression was tested by staining of the BSD-resistant population with anti-human Fc antibody. The antibodies integrated in the NLN locus show homogenous expression (FIG. 3B).

### Example 4: Validation of the NLN Locus in CHO Cells

The aim of this example was to test stable surface antibody presentation from neurolysin (NLN) intron 1 loci in CHO-s cells.

### Experimental Procedures

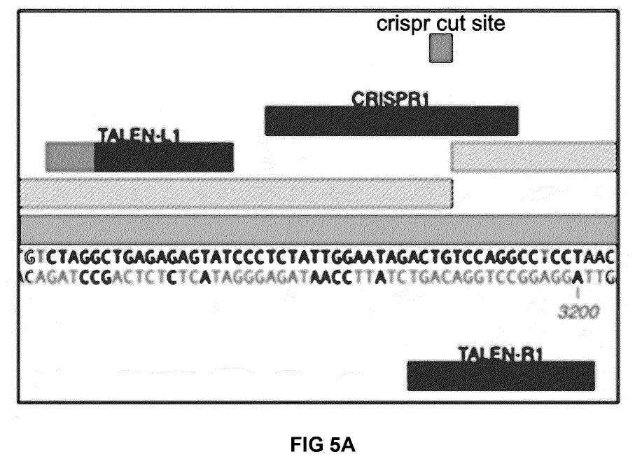

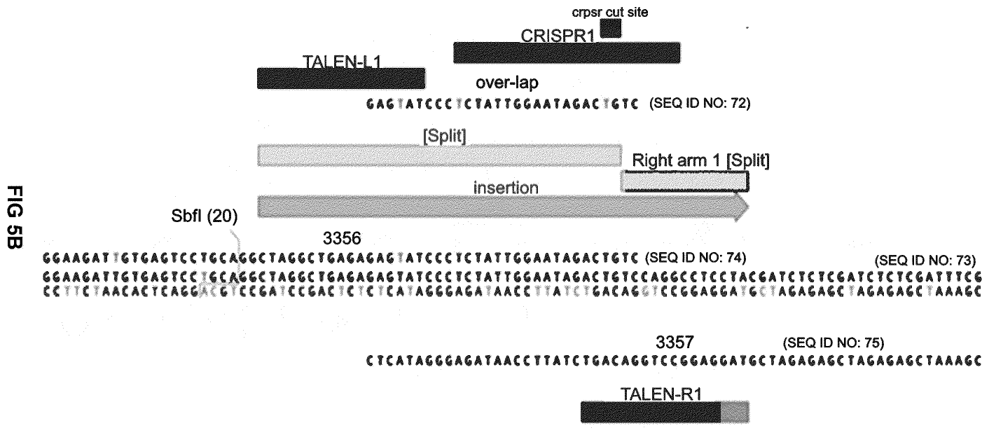

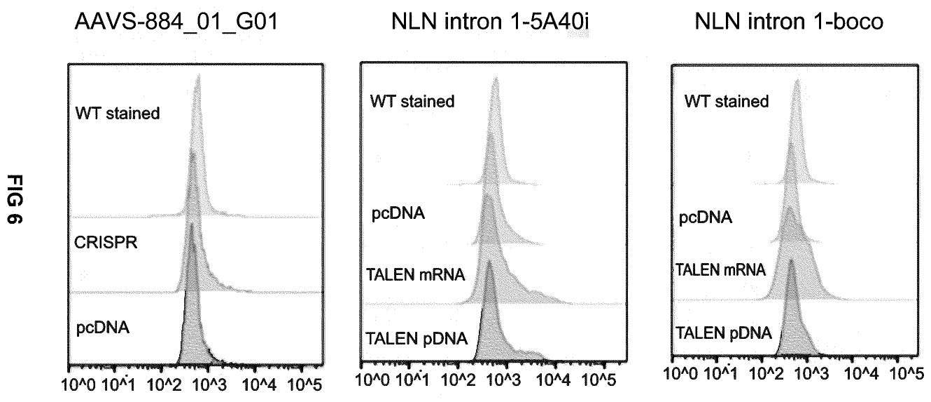

Integration of antibody genes was performed in the AAVS locus using CRISPR/Cas9 (as described in WO2019/110691) or in NLN intron 1 using TALEN (SEQ ID NO: 66). CHO-s cells were co-transfected with a targeting plasmid harboring a variant bococizumab antibody gene (pINT157-884_01_G01 (SEQ ID NO: 67), described in Dyson et al. 2020 mAbs, 12:1, 1829335, for AAVS-targeting. CHO-s cells were co-transfected with targeting plasmids harboring bococizumab and 5A10i (pINT158-bococizumab (SEQ ID NO: 68) and PINT158-5A10i (SEQ ID NO: 69) for NLN intron 1 targeting. 5A10i and Bococizumab represent well- and poorly-behaved antibodies respectively, therefore differential display of the antibodies was expected. This can be assessed by changes in the magnitude of signal (e.g., MFI) detected by a secondary-fluorescent antibody directed at Fc regions of the displayed antibody (anti-human Fc antibody, described in Example 3). The experiment was done in duplicate in the case of transfections with TALEN pDNA (see Tables 8 and 10). FIGS. 4A and 4B show the NLN gene structure in CHO cells and FIGS. 5A and 5B show the NLN intron 1 TALEN targeting design.

### Cell Culture, Flow Cytometry and Staining

All CHO-S cell lines were cultivated in CD Opticho Media (for CHO-F cells; catalogue no. 12681-011, Life Technologies, California, USA) supplemented with 8 mM L-Glut (catalogue no. 25030-024, Life Technologies, California, USA) and typically maintained in 25 ml culture passaged every 72/96 hours. BSD (blasticidin) concentration for selection was 3 μg/ml. To estimate transgene integration efficiency, a duplicate culture without BSD selection was also maintained.

Anti-human Fc antibody cell staining and flow-cytometric analysis was performed as described in Example 3.

### Transfections

Transfections were performed using Maxcyte (MD, USA) transfection following the manufacturer's protocol (see also Table 6). TALEN-mediated integration was performed as described in WO2015/166272. The transfection plan is summarized in Table 8 below:

TABLE 8
Transfection plan summary. pcDNA 3.0 was used
in negative control transfections
(no nuclease). TALEN mRNA refers to
donor DNA + TALEN mRNA and
TALEN pDNA to donor DNA + TALEN plasmid.

| Reaction | Donor DNA | Locus | Integration method |
|---|---|---|---|
| 1 | pINT157-884_01_G01 | AAVS | CRISPR |
| 2 | pINT157-884_01_G01 | AAVS | pcDNA 3.0 |
| 3 | pINT158-5A10i | NLN intron 1 | TALEN pDNA |
| 4 | pINT158-5A10i | NLN intron 1 | TALEN mRNA |
| 5 | pINT158-5A10i | NLN intron 1 | pcDNA 3.0 |
| 6 | pINT158-Bococizumab | NLN intron 1 | TALEN pDNA |
| 7 | pINT158-Bococizumab | NLN intron 1 | TALEN mRNA |
| 8 | pINT158-Bococizumab | NLN intron 1 | pcDNA 3.0 |

### Results

### Anti-Human Fc Antibody Staining 2 Days Post Transfection

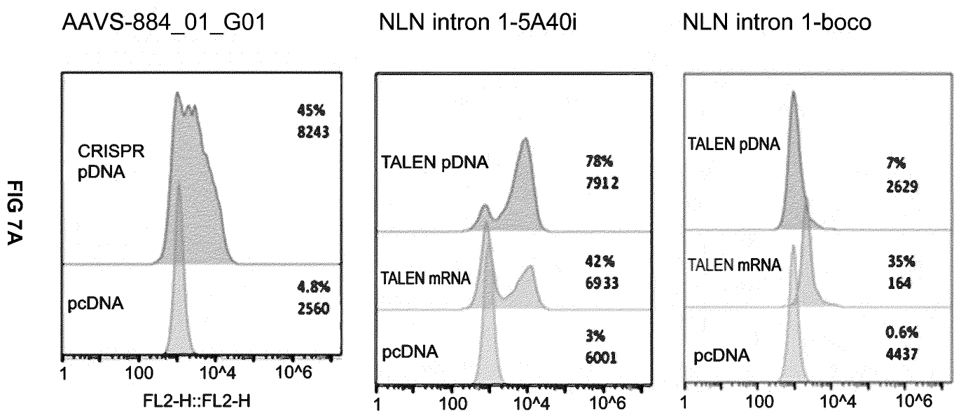

2 days post transfection samples were stained with an anti-human Fc antibody for detection of transient antibody expression. Samples transfected with TALEN mRNA showed increased expression over both pcDNA and TALEN pDNA, suggesting early integration/expression of antibody-gene cassettes. A difference in expression profiles between 5A10i and bococizumab was also observed as expected due to the ‘good’ and ‘bad’ presentation profiles of the antibodies, respectively (FIG. 6).

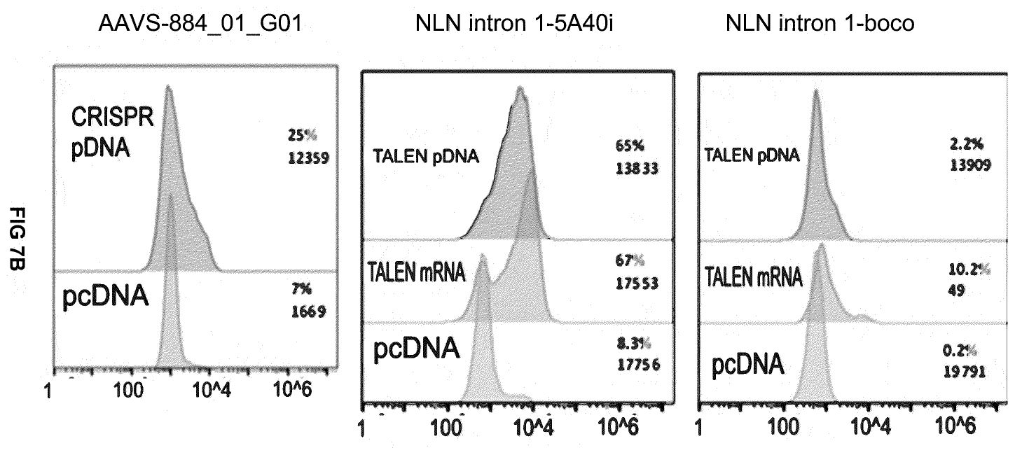

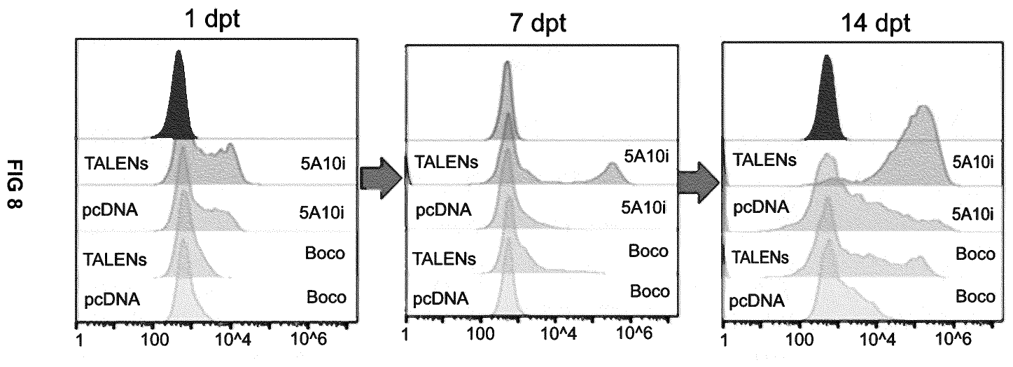

Anti-human Fc antibody staining 8 days post transfection (BSD-resistant cells) 8 days post transfection, samples taken from cells cultured in the presence of BSD were stained with an anti-human Fc antibody for detection of antibody cell surface expression. 8 days post transfection is still an early time-point for generating a clean, stably expressing cell line as cells are still under antibiotic selection. Despite this, a fairly clean population of >78% anti-human Fc antibody positive cells was observed in cells wherein 5A10i was integrated in NLN intron 1, in contrast to bococizumab which only displayed >7%, likely due to the inherent bad display properties of the antibody (FIG. 7A). As expected, samples taken from transfections using pcDNA showed little to no expression in the absence of nuclease-mediated gene integration.

### Anti-Human Fc Antibody Staining 14 Days Post Transfection (BSD-Resistant Cells)

14 days post transfection samples taken from cells cultured in the presence of BSD were stained with an anti-human Fc antibody for detection of antibody cell surface expression. A clear shift of the whole population was observed for in cells wherein 5A10i was integrated in NLN intron 1 (FIG. 7B). This confirmed the utility of targeting antibody expression to NLN intron 1. There was a clear differential expression between 5A10i and bococizumab confirming developability of cell-surface display based on antibody biophysical properties.

Integration Efficiency 8 Days and 14 Days Post Transfection (without BSD Selection)

Samples taken from cells cultured without the presence of BSD were stained with an anti-human Fc antibody for integration efficiency measurements at 8- and 14-days post transfection. The results are given in Table 9 below:

TABLE 9
Integration efficiency measurements in samples taken from
cells cultured without the presence of
BSD. Numbers refer to % of positive cells within the gate.

| | | | % positive | % positive |
|---|---|---|---|---|
| | | | cells 8 days | cells 14 days |
| | | Integration | post | post |
| Donor DNA | Locus | method | transfection | transfection |
| pINT157- | AAVS | CRISPR | 0.29 | 0.24 |
| 884_01_G01 | | | | |
| pINT157- | AAVS | pcDNA 3.0 | 0.20 | 0.12 |
| 884_01_G01 | | | | |
| pINT158-5A10i | NLN | TALEN | 2.41 | 1.05 |
| | intron 1 | pDNA | | |
| pINT158-5A10i | NLN | TALEN | 2.64 | 1.16 |
| | intron 1 | mRNA | | |
| pINT158-5A10i | NLN | pcDNA 3.0 | 0.12 | 0.15 |
| | intron 1 | | | |
| pINT158- | NLN | TALEN | 0.46 | 0.06 |
| Bococizumab | intron 1 | pDNA | | |
| pINT158- | NLN | TALEN | 0.25 | 0.04 |
| Bococizumab | intron 1 | mRNA | | |
| pINT158- | NLN | pcDNA 3.0 | 0.07 | 0.06 |
| Bococizumab | intron 1 | | | |

At 8 days post transfection, the measurements from samples taken from cells cultured without BSD stained with anti-human Fc antibody represent an approximate number for antibody-gene integration efficiency. Based on the 5A10i antibody, a ˜2.5% integration efficiency with both TALEN plasmid DNA and TALEN mRNA was achieved in the case of the design targeting intron 1 of NLN.

### Repeat Transfection Experiment:

The following transfections were repeated to check the reproducibility of targeting intron 1 of NLN (Table 10):

TABLE 10
Repeat transfection summary. pcDNA 3.0 was used in negative
control transfections (no nuclease). TALEN pDNA refers
to donor DNA + TALEN plasmid.

| Reaction | Donor DNA | Locus | Integration method |
|---|---|---|---|
| 3 | pINT158-5A10i | NLN-Design 1 | TALENs pDNA |
| 5 | pINT158-5A10i | NLN-Design 1 | pcDNA |
| 6 | pINT158-Bococizumab | NLN-Design 1 | TALENs pDNA |
| 8 | pINT158-Bococizumab | NLN-Design 1 | pcDNA |

### BSD-Resistant Cells

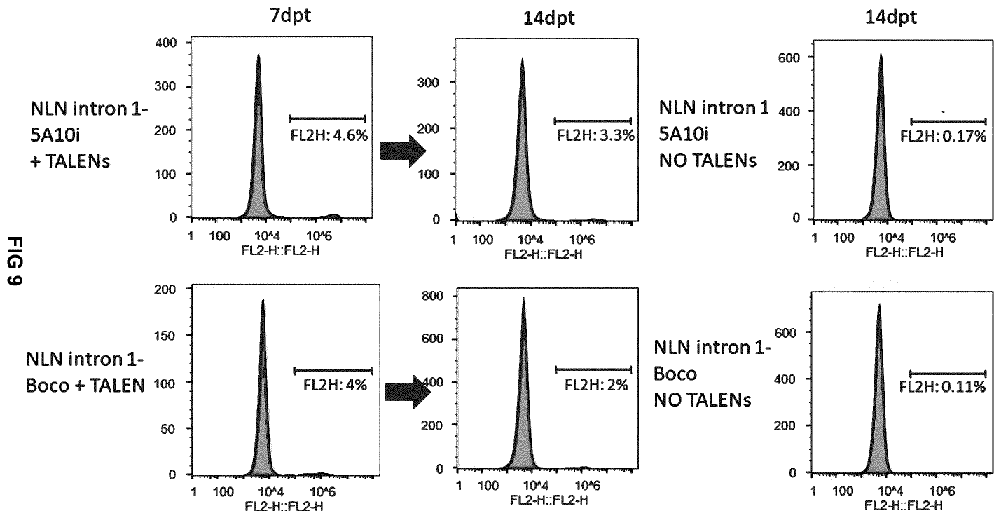

Samples taken from cell cultures in the presence of BSD were stained using an anti-human Fc antibody at 1-, 7- and 14-days post transfection. Expression profiles of bococizumab and 5A10i show differential expression based on the inherent biophysical properties of the two antibodies. The results confirm the reproducibility and utility of targeting antibody expression for mammalian display (FIG. 8).

Integration Efficiency Measured at 7 Days Post Transfection and 14 Days Post Transfection (without BSD Selection)

Samples taken from cell cultures without the presence of BSD were stained for integration efficiency measurements at 7- and 14-days post transfection with an anti-human Fc antibody. Numbers refer to % of positive cells within the gate. Integration efficiency of up to 4.6% were achieved at 7 days post transfection which was still detectable at 3.3% at 14 days post transfection (FIG. 9). These numbers confirm the integration efficiency previously observed in the design targeting intron 1 of NLN.

### REFERENCES

All references listed below and others cited anywhere in this disclosure are incorporated herein by reference in their entirety.
1 Russell, S. et al. (1993). Retroviral vectors displaying functional antibody fragments. Nucleic Acids Res, 21 (5), 1081-1085.
2 Boublik, Y et al. (1995). Eukaryotic virus display: Engineering the major surface glycoprotein of the Autographa californica nuclear polyhedrosis virus (AcNPV) for the presentation of foreign proteins on the virus surface. Nature Biotechnology, 13 (10), 1079-1084.
3 Mottershead, D. G. et al (2000). Baculoviral display of functional scFv and synthetic IgG-binding domains. Biochemical and Biophysical Research Communications, 275 (1), 84-90.
4 Oker-Blom, C. et al. (2003). Baculovirus display strategies: Emerging tools for eukaryotic libraries and gene delivery. Briefings in Functional Genomics and Proteomics, 2 (3), 244-253.
5 Edwards, B. M. et al. (2003). The Remarkable Flexibility of the Human Antibody Repertoire; Isolation of Over One Thousand Different Antibodies to a Single Protein, BLyS. Journal of Molecular Biology, 334 (1), 103-118. doi: 10.1016/j.jmb. 2003.09.054
6 Pershad, K. et al. (2010). Generating a panel of highly specific antibodies to 20 human SH2 domains by phage display. Protein Engineering, Design and Selection, 23 (4), 279-288. doi: 10.1093/proteinlgzq003
7 Schofield, D. J. et al. (2007). Application of phage display to high throughput antibody generation and characterization. Genome Biol, 8 (11), R254.
8 Salema, V. et al. (2013). Selection of Single Domain Antibodies from Immune Libraries Displayed on the Surface of E. coli Cells with Two P-Domains of Opposite Topologies. PLOS ONE, 8 (9), e75126. doi: 10.1371/journal. pone. 0075126.s007
9 Chao, G. et al. (2006). Isolating and engineering human antibodies using yeast surface display. Nat Protoc, 1 (2), 755-768.
10 Higuchi, K. et al. (1997). Cell display library for gene cloning of variable regions of human antibodies to hepatitis B surface antigen. J Immunol Methods, 202 (2), 193-204.
11 Ho, M. et al. (2006). Isolation of anti-CD22 Fv with high affinity by Fv display on human cells. Proc Natl Acad Sci USA, 103 (25), 9637-9642. doi: 10.1073/pnas. 0603653103
12 Ho, M. et al. (2009). Display and selection of scFv antibodies on HEK-293T cells. Methods Mol Biol, 562, 99-113. doi: 10.1007/978-1-60327-302-2 8
13 Akamatsu, Y., Pakabunto, K., Xu, Z., Zhang, Y., & Tsurushita, N. (2007). Whole IgG surface display on mammalian cells: Application to isolation of neutralizing chicken monoclonal anti-IL-12 antibodies. Journal of Immunological Methods, 327 (1-2), 40-52. doi: 10.1016/j.jim. 2007.07.007
14 Beerli, R. R., Bauer, M., Buser, R. B., Gwerder, M., Muntwiler, S., Maurer, P., et al. Isolation of human monoclonal antibodies by mammalian cell display. Proc Natl Acad Sci USA. 2008 Sep. 23; 105 (38): 14336-41. doi: 10.1073/pnas. 0805942105
15 Breous-Nystrom, E., Schultze, K., Meier, M., Flueck, L., Holzer, C., Boll, M., et al. (2013). Retrocyte Display@ technology: Generation and screening of a high diversity cellular antibody library. Methods, 1-11. doi: 10.1016/j.ymeth. 2013.09.003
16 Grindley, Whiteson & Rice. Mechanisms of Site-Specific Recombination. Annu Rev Biochem 2006 75:567-605
17 Zhou, C., Jacobsen, F. W., Cai, L., Chen, Q., Shen, W. D. (2010). Development of a novel mammalian cell surface antibody display platform. mAbs, 2 (5), 508-518
18 Li, C. Z., Liang, Z. K., Chen, Z. R., Lou, H. B., Zhou, Y., Zhang, Z. H., et al. (2012). Identification of HBsAg-specific antibodies from a mammalian cell displayed full-length human antibody library of healthy immunized donor. Cellular and Molecular Immunology, 9 (2), 184-190.
19 Buchholz, F., Ringrose, L., Angrand, P. O., Rossi, F., & Stewart, A. F. (1996). Different thermostabilities of FLP and Cre recombinases: Implications for applied site-specific recombination. Nucleic Acids Res, 24 (21), 4256-4262.
20 Schaft, J., Ashery-Padan, R., Van Hoeven, F. D., Gruss, P., & Francis Stewart, A. (2001). Efficient FLP recombination in mouse ES cells and oocytes. Genesis, 37 (1), 6-10.
21 Moehle, E. A., Moehle, E. A., Rock, J. M., Rock, J. M., Lee, Y.-L., Lee, Y. L., et al. (2007). Targeted gene addition into a specified location in the human genome using designed zinc finger nucleases. Proc Natl Acad Sci USA, 104 (9), 3055-3060. doi: 10.1073/pnas. 0611478104
22 Cristea, S., Freyvert, Y., Santiago, Y., Holmes, M. C., Urnov, F. D., Gregory, P. D., Cost, G. J. (2013). In vivo cleavage of transgene donors promotes nuclease-mediated targeted integration. Biotechnology and Bioengineering/10 (3), 871-880. doi: 10.1002/bit. 24733
23 Letourneur, F., Malissen, B. (1989). Derivation of a T cell hybridoma variant deprived of functional T cell receptor alpha and beta chain transcripts reveals a nonfunctional alpha-mRNA of BW5147 origin. European Journal of Immunology, 19 (12), 2269-2274. doi: 10.1002/eji. 1830191214
24 Kanayama, N., Todo, K., Reth, M., Ohmori, H. (2005). Reversible switching of immunoglobulin hypermutation machinery in a chicken B cell line. Biochem Biophys Res Commun, 327 (1), 70-75. doi: 10.1016/j.bbrc. 2004.11.143
25 Lin, W., Kurosawa, K., Murayama, A., Kagaya, E., 8 Ohta, K. (2011). B-cell display-based one-step method to generate chimeric human IgG monoclonal antibodies. Nucleic Acids Research, 39 (3), e14-e14. doi: 10.1093/nar/gkq1122
26 Adachi, N., So, S., liizumi, S., Nomura, Y., Murai, K., Yamakawa, C., et al. (2006). The human pre-B cell line Nalm-6 is highly proficient in gene targeting by homologous recombination. DNA and Cell Biology, 25 (1), 19-24. doi: 10.1089/dna. 2006.25.19
27 Palacios, R., 8 Steinmetz, M. (1985). IL3-Dependent mouse clones that express B-220 surface antigen, contain Ig genes in germ-line configuration, and generate B lymphocytes in vivo. Cell, 41 (3), 727-734
28 Barretina, J., Caponigro, G., Stransky, N., Venkatesan, K., Margolin, A. A., Kim, S., et al. (2012). The Cancer Cell Line Encyclopedia enables predictive modelling of anticancer drug sensitivity. Nature, 483 (7391), 603-607. doi: 10.1038/nature11003
29 Forbes, S. A., Bindal, N., Bamford, S., Cole, C., Kok, C. Y., Beare, D., et al. (2011). COSMIC: mining complete cancer genomes in the Catalogue of Somatic Mutations in Cancer. Nucleic Acids Research, 39 (Database issue), D945-50. doi: 10.1093/nar/gkq929
30 Silva, G., Poirot, L., Galetto, R., Smith, J., Montoya, G., Duchateau, P., Paques, F. (2011). Meganucleases and other tools for targeted genome engineering: Perspectives and challenges for gene therapy. Current Gene Therapy, 1′/(1), 11-27
31 Epinat, J. C., Silva, G. H., Paques, F., Smith, J., Duchateau, P. (2013). Engineered meganucleases for genome engineering purposes. Topics in Current Genetics (Vol. 23, pp. 147-185).
32 Szczepek, M., Brondani, V., Buchel, J., Serrano, L., Segal, D. J., Cathomen, T. (2007). Structure-based redesign of the dimerization interface reduces the toxicity of zinc-finger nucleases. Nature Biotechnology, 25 (7), 786-793. doi: 10.1038/nbt1317
33 Doyon, Y., Vo, T. D., Mendel, M. C., Greenberg, S. G., Wang, J., Xia, D. F., et al. (2011). Enhancing zinc-finger-nuclease activity with improved obligate heterodimeric architectures. Nat Methods, 8 (1), 74-79. doi: 10.1038/nmeth. 1539
34 Perez-Pinera, P., Ousterout, D. G., Brown, M. T., Gersbach, C. A. (2012). Gene targeting to the ROSA26 locus directed by engineered zinc finger nucleases. Nucleic Acids Research, 40 (8), 3741-3752. doi: 10.1093/nar/gkr1 214
35 Urnov, F. D., Miller, J. C., Lee, Y.-L., Beausejour, C. M., Rock, J. M., Augustus, S., et al. (2005). Highly efficient endogenous human gene correction using designed zinc-finger nucleases. Nature, 435 (7042), 646-651. doi: 10.1038/nature03556
36 Maresca, M., Lin, V. G., Guo, N., & Yang, Y. (2013). Obligate ligation-gated recombination (ObLiGaRe): custom-designed nuclease-mediated targeted integration through nonhomologous end joining. Genome Research, 23 (3), 539-546. doi: 10.1101/gr. 145441.112
37 Bogdanove, A. J., & Voytas, D. F. (2011). TAL effectors: customizable proteins for DNA targeting. Science, 333 (6051), 1843-1846. doi: 10.1126/science. 1204094
38 Reyon, D., Tsai, S. Q., Khgayter, C., Foden, J. A., Sander, J. D., 8 Joung, J. K. (2012). FLASH assembly of TALENs for high-throughput genome editing. Nature Biotechnology, 30 (5), 460-465. doi: 10.1038/nbt. 2170
39 Boissel, S., Jarjour, J., Astrakhan, A., Adey, A., Gouble, A., Duchateau, P., et al. (2013). megaTALs: a rare-cleaving nuclease architecture for therapeutic genome engineering. Nucleic Acids Research. doi: 10.1093/nar/gkt1224
40 Beurdeley, M., Bietz, F., Li, J., Thomas, S., Stoddard, T., Juillerat, A., et al. (2013). Compact designer TALENs for efficient genome engineering. Nature Communications, 4, 1762. doi: 10.1038/ncomms2782
41 Sampson, T. R., Weiss, D. S. (2014). Exploiting CRISPR/Cas systems for biotechnology. BioEssays: News and Reviews in Molecular, Cellular and Developmenta/Biology, 36 (1), 34-38. doi: 10.1002/bies. 201300135
42 Shalem, O., Sanjana, N. E., Hartenian, E., Shi, X., Scott, D. A., Mikkelsen, T. S., et al. (2014). Genome-scale CRISPR-Cas9 knockout screening in human cells. Science, 343 (6166), 84-87. doi: 10.1126/science. 1247005
43 Wang, T., Wei, J. J., Sabatini, D. M., Lander, E. S. (2014). Genetic screens in human cells using the CRISPR-Cas9 system. Science, 343 (6166), 80-84. doi: 10.1126/science.246981
44 Beard, C., Hochedlinger, K., Plath, K., Wutz, A., & Jaenisch, R. (2006). Efficient method to generate single-copy transgenic mice by site-specific integration in embryonic stem cells. Genesis, 44 (1), 23-28.
45 Orlando, S. J., Santiago, Y., DeKelver, R. C., Freyvert, Y., Boydston, E. A., Moehle, E. A., et al. (2010). Zinc-finger nuclease-driven targeted integration into mammalian genomes using donors with limited chromosomal homology. Nucleic Acids Research, 38 (15), e152. doi: 10.1093/nar/gkq 512
46 Cadinanos 2007
47 Zhang, H., Yea, K., Xie, J., Ruiz, D., Wilson, I. A., Lerner, R. A. (2013). Selecting agonists from single cells infected with combinatorial antibody libraries. Chemistry 8 Biology, 20 (5), 734-741. doi: 10.1016/j.chembiol. 2013.04.012
48 Porteus, M. H., Baltimore, D. (2003). Chimeric nucleases stimulate gene targeting in human cells. Science, 300 (5620), 763. doi: 10.1126/science. 1078395
49 Rouet, P., Smih, F., & Jasin, M. (1994). Introduction of double-strand breaks into the genome of mouse cells by expression of a rare-cutting endonuclease. Molecular and Cellular Biology, 14 (12), 8096-8106.
50 Jasin, M. (1996). Genetic manipulation of genomes with rare-cutting endonucleases. Trends in Genetics, 12 (6), 224-228
51 Davis, L., & Maizels, N. (2011). DNA nicks promote efficient and safe targeted gene correction. PLOS ONE, 6 (9), e23981. doi: 10.1371/journal. pone. 0023981
52 Fujioka, K., Aratani, Y., Kusano, K., Koyama, H. (1993). Targeted recombination with single-stranded DNA vectors in mammalian cells. Nucleic Acids Res, 21 (3), 407-412
53 Khan, I. F., Hirata, R. K., & Russell, D. W. (2011). AAV-mediated gene targeting methods for human cells. Nat Protoc, 6 (4), 482-501. doi: 10.1038/nprot. 2011.301
54 Deyle, D. R., 8 Russell, D. W. (2009). Adeno-associated virus vector integration. Current Opinion in Molecular Therapeutics, 11 (4), 442-447
55 Benatuil, L., Perez, J. M., Belk, J., Hsieh, C. M. (2010). An improved yeast transformation method for the generation of very large human antibody libraries. Protein Engineering, Design and Selection, 23 (4), 155-159.
56 Feldhaus, M. J., Siegel, R. W., Opresko, L. K., Coleman, J. R., Feldhaus, J. M., Yeung, Y. A., et al. (2003). Flow-cytometric isolation of human antibodies from a nonimmune Saccharomyces cerevisiae surface display library. Nat Biotechnol.
57 Zhao, A., Nunez-Cruz, S., Li, C., Coukos, G., Siegel, D. L., 8 Scholler, N. (2011). Rapid isolation of high-affinity human antibodies against the tumor vascular marker Endosialin/TEM1, using a paired yeast-display/secretory scFv library platform. Journal of Immunological Methods, 363 (2), 221-232. doi: 10.1016/j.jim. 2010.09.001
58 Skerra, A. (2007). Alternative non-antibody scaffolds for molecular recognition. Curr Opin Biotechnol, 18 (4), 295-304. doi: 10.1016/j.copbio. 2007.04.010
59 Gebauer, M., & Skerra, A. (2009). Engineered protein scaffolds as next-generation antibody therapeutics. Current Opinion in Chemical Biology, 13 (3), 245-255. doi: 10.1016/j.cbpa. 2009.04.627
60 Haan & Maggos (2004) BioCentury, 12 (5): A1-A6
61 Koide et al. (1998) Journal of Molecular Biology, 284:1141-1151.
62 Nygren et al. (1997) Current Opinion in Structural Biology, 7:463-469
63 Wess, L. In: BioCentury, The Bernstein Report on BioBusiness, 12 (42), A1-A7, 2004
64 Chang, H.-J., Hsu, H.-J., Chang, C.-F., Peng, H.-P., Sun, Y.-K., Yu, H.-M., et al. (2009). Molecular Evolution of Cystine-Stabilized Miniproteins as Stable Proteinaceous Binders. Structure, 17 (4), 620-631. doi: 10.1016/j.str. 2009.01.011
65 Ward, E. S. et al., Nature 341, 544-546 (1989)
66 McCafferty et al Nature, 348, 552-554 (1990)
67 Holt et al Trends in Biotechnology 21, 484-490 (2003)
68 Bird et al, Science, 242, 423-426, (1988)
69 Huston et al, PNAS USA, 85, 5879-5883, (1988)
70 Holliger, P. et al, PNAS USA 90 6444-6448, (1993)
71 Reiter, Y. et al, Nature Biotech, 14, 1239-1245, (1996)
72 Holliger & Hudson, Nature Biotechnology 23 (9): 1126-1136 (2005)
73 Knappik et al. J. Mol. Biol. 296, 57-86 (2000)
74 Krebs et al. Journal of Immunological Methods 254, 67-84 (2001)
75 Holliger and Bohlen Cancer and metastasis rev. 18:411-419 (1999)
76 Holliger, P. and Winter G. Current Opinion Biotechnol 4, 446-449 (1993)
77 Glennie M J et al., J. Immunol. 139, 2367-2375 (1987)
78 Repp R. et al., J. Hemat. 377-382 (1995)
79 Staerz U. D. and Bevan M. J. PNAS 83 (1986)
80 Suresh M. R. et al., Method Enzymol. 121:210-228 (1986)
81 Merchand et al., Nature Biotech. 16:677-681 (1998)
82 Ridgeway, J. B. B. et al, Protein Eng., 9, 616-621, (1996)
83 Marks, J. D., Hoogenboom, H. R., Bonnert, T. P., McCafferty, J., Griffiths, A. D., & Winter, G. (1991). By-passing immunization. Human antibodies from V-gene libraries displayed on phage. J Mol Biol, 222 (3), 581-597.
84 Gronwald, R. G. K., Grant, F. J., Haldeman, B. A., Hart, C. E., O'Hara, P. J., Hagen, F. S., et al. (1988). Cloning and expression of a cDNA coding for the human platelet-derived growth factor receptor: Evidence for more than one receptor class (Vol. 85, pp. 3435-3439). Presented at the Proceedings of the National Academy of Sciences of the United States of America.
85 Kumar, N., & Borth, N. (2012). Flow-cytometry and cell sorting: an efficient approach to investigate productivity and cell physiology in mammalian cell factories. Methods, 56 (3), 366-374. doi: 10.1016/j.ymeth. 2012.03.004
86 Brezinsky, S. C. G., Chiang, G. G., Szilvasi, A., Mohan, S., Shapiro, R. I., MacLean, A., et al. (2003). A simple method for enriching populations of transfected CHO cells for cells of higher specific productivity. J Immuno/Methods, 277 (1-2), 141-155.
87 Pichler, J., Hesse, F., Wieser, M., Kunert, R., Galosy, S. S., Mott, J. E., & Borth, N. (2009). A study on the temperature dependency and time course of the cold capture antibody secretion assay. Journal of Biotechnology, 141 (1-2), 80-83. doi: 10.1016/j.jbiotec. 2009.03.001
88 Anastassiadis, K., Fu, J., Patsch, C., Hu, S., Weidlich, S., Duerschke, K., et al. (2009). Dre recombinase, like Cre, is a highly efficient site-specific recombinase in E. coli, mammalian cells and mice. Disease Models & Mechanisms, 2 (9-10), 508-515. doi: 10.1242/dmm. 003087
89 Horlick, R. A., Macomber, J. L., Bowers, P. M., Neben, T. Y., Tomlinson, G. L., Krapf, I. P., et al. (2013). Simultaneous surface display and secretion of proteins from mammalian cells facilitate efficient in vitro selection and maturation of antibodies. J Biol Chem, 288 (27), 19861-19869. doi: 10.1074/jbc. M113.452482
90 Biffi, G., Tannahill, D., McCafferty, J., & Balasubramanian, S. (2013). Quantitative visualization of DNA G-quadruplex structures in human cells. Nature Chemistry, 5 (3), 182-186. doi: 10.1038/nchem. 1548
91 Gao, J., Sidhu, S. S., & Wells, J. A. (2009). Two-state selection of conformation-specific antibodies. Proc Natl Acad Sci USA, 106 (9), 3071-3076. doi: 10.1073/pnas. 0812952106
92 Gu, G. J., Friedman, M., Jost, C., Johnsson, K., Kamali-Moghaddam, M., Pluckthun, A., et al. (2013). Protein tag-mediated conjugation of oligonucleotides to recombinant affinity binders for proximity ligation. N Biotechno/, 30 (2), 144-152. doi: 10.1016/j.nbt. 2012.05.005
93 Cho, Y. K., & Shusta, E. V. (2010). Antibody library screens using detergent-solubilized mammalian cell lysates as antigen sources. Profein Eng Des Sei, 23 (7), 567-577. doi: 10.1093/protein/gzq 029
94 Tillotson, B. J., Cho, Y. K., & Shusta, E. V. (2013). Cells and cell lysates: a direct approach for engineering antibodies against membrane proteins using yeast surface display. Methods, 60 (1), 27-37. doi: 10.1016/j.ymeth. 2012.03.010
95 Kunert, A., Straetemans, T., Govers, C., Lamers, C., Mathijssen, R., Sleijfer, S., Debets, R. (2013). TCR-Engineered T Cells Meet New Challenges to Treat Solid Tumors: Choice of Antigen, T Cell Fitness, and Sensitization of Tumor Milieu. Frontiersin Immunology, 4, 363. doi: 10.3389/fimmu. 2013.00363
96 Liddy, N., Bossi, G., Adams, K. J., Lissina, A., Mahon, T. M., Hassan, N. J., et al. (2012). Monoclonal TCR-redirected tumor cell killing. Nature Medicine, 18 (6), 980-987. doi: 10.1038/nm. 2764
97 Holler, P. D., Holman, P. O., Shusta, E. V., O'Herrin, S., Wittrup, K. D., 8 Kranz, D. M. (2000). In vitro evolution of a T cell receptor with high affinity for peptide/MHC. Proc Natl Acad Sci USA, 97 (10), 5387-5392. doi: 10.1073/pnas. 080078297
98 Weber, K. S., Donermeyer, D. L., Allen, P. M., 8 Kranz, D. M. (2005). Class II-restricted T cell receptor engineered in vitro for higher affinity retains peptide specificity and function. Proc Natl Acad Sci USA, 102 (52), 19033-19038. doi: 10.1073/pnas. 0507554102
99 Kessels, H. W., van Den Boom, M. D., Spits, H., Hooijberg, E., 8 Schumacher, T. N. (2000). Changing T cell specificity by retroviral T cell receptor display. Proc Natl Acad Sci USA, 97 (26), 14578-14583. doi: 10.1073/pnas. 97.26.14578
100 Chervin, A. S., Aggen, D. H., Raseman, J. M., 8 Kranz, D. M. (2008). Engineering higher affinity T cell receptors using a T cell display system. Journal of Immunological Methods, 339 (2), 175-184.
101 Crawford, F., Jordan, K. R., Stadinski, B., Wang, Y., Huseby, E., Marrack, P., et al. (2006). Use of baculovirus MHC/peptide display libraries to characterize T-cell receptor ligands. Immunological Reviews, 210, 156-170. doi: 10.1111/j.0105-2896.2006.00365.x
102 Hinrichs, C. S., K Restifo, N. P. (2013). Reassessing target antigens for adoptive T-cell therapy. Nat Biotechnol, 31 (11), 999-1008. doi: 10.1038/nbt. 2725
103 Sadelain, M., Brentjens, R., 8 Riviere, I. (2013). The basic principles of chimeric antigen receptor design. Cancer Discovery, 3 (4), 388-398. doi: 10.1158/2159-8290.CD-12-0548
104 Alonso-Camino, V., Sanchez-Martin, D., Compte, M., Sanz, L., 8 Alvarez-Vallina, L. (2009). Lymphocyte display: a novel antibody selection platform based on T cell activation. PLOS ONE, 4 (9), e7174. doi: 10.1371/journal. pone. 0007174
105 Melidoni, A. N., Dyson, M. R., Wormald, S., 8 McCafferty, J. (2013). Selecting antagonistic antibodies that control differentiation through inducible expression in embryonic stem cells. Proceedings of the National Academy of Sciences, 110 (44), 17802-17807. doi: 10.1073/pnas. 1312062110
106 Zhang, H., Wilson, I. A., & Lerner, R. A. (2012). Selection of antibodies that regulate phenotype from intracellular combinatorial antibody libraries. Proceedings of the National Academy of Sciences, 109 (39), 15728-1 5733. doi: 10.1073/pnas. 1214275109
107 Xie, J., Yea, K., Zhang, H., Moldt, B., He, L., Zhu, J., 8 Lerner, R. A. (2014). Prevention of cell death by antibodies selected from intracellular combinatorial libraries. Chemistry & Biology, 2/(2), 274-283.
108 Yea, K., Zhang, H., Xie, J., Jones, T. M., Yang, G., Song, B. D., 8 Lerner, R. A. (2013). Converting stem cells to dendritic cells by agonist antibodies from unbiased morphogenic selections (Vol. 110, pp. 14966-14971). Presented at the Proceedings of the National Academy of Sciences of the United States of America. doi: 10.1073/pnas. 1313671110
109 Kawahara, M., Kimura, H., Ueda, H., 8 Nagamune, T. (2004). Selection of genetically modified cell population using hapten-specific antibody/receptor chimera. Biochem Biophys Res Commun, 3′/5 (1), 132-138. doi: 10.1016/j.bbrc. 2004.01.030
110 Kawahara, M., Shimo, Y., Sogo, T., Hitomi, A., Ueda, H., 8 Nagamune, T. (2008). Antigenmediated migration of murine pro-B Ba/F3 cells via an antibody/receptor chimera. Journal of Biotechnology, 133 (1), 154-161. doi: 10.1016/j.jbiotec. 2007.09.009
111 Sogo, T., Kawahara, M., Ueda, H., Otsu, M., Onodera, M., Nakauchi, H., 8 Nagamune, T. (2009). T cell growth control using hapten-specific antibody/interleukin-2 receptor chimera. Cytokine, 46 (1), 127-1 36. doi: 10.1016/j.cyto. 2008.12.020
112 Kawahara, M., Chen, J., Sogo, T., Teng, J., Otsu, M., Onodera, M., et al. (2011). Growth promotion of genetically modified hematopoietic progenitors using an antibody/c-Mpl chimera. Cytokine, 55 (3), 402-408. doi: 10.1016/j.cyto. 2011.05.024
113 Ueda, H., Kawahara, M., Aburatani, T., Tsumoto, K., Todokoro, K., Suzuki, E., et al. (2000). Cell-growth control by monomeric antigen: the cell surface expression of lysozyme-specific Ig Vdomains fused to truncated Epo receptor. J Immuno/Methods, 241 (1-2), 159-170.
114 Kerppola, T. K. (2009). Visualization of molecular interactions using bimolecular fluorescence complementation analysis: Characteristics of protein fragment complementation. Chemical Society Reviews, 38 (10), 2876-2886.
115 Michnick, S. W., Ear, P. H., Manderson, E. N., Remy, I., 8 Stefan, E. (2007). Universal strategies in research and drug discovery based on protein-fragment complementation assays. Nature Reviews Drug Discovery, 6 (7), 569-582. doi: 10.1038/nrd231
116 Petschnigg, J., Groisman, B., Kotlyar, M., Taipale, M., Zheng, Y., Kurat, C. F., et al. (2014). The mammalian-membrane two-hybrid assay (MaMTH) for probing membrane-protein interactions in human cells. Nat Methods. doi: 10.1038/nmeth. 2895
117 Renaut, L., Monnet, C., Dubreuil, O., Zaki, O., Crozet, F., Bouayadi, K., et al. (2012). Affinity maturation of antibodies: Optimized methods to generate high-quality scfv libraries and isolate igg candidates by high-throughput screening. Methods in Molecular Biology (Vol. 907, pp. 451-461)
118 Dyson, M. R., Zheng, Y., Zhang, C., Colwill, K., Pershad, K., Kay, B. K., et al. (2011). Mapping protein interactions by combining antibody affinity maturation and mass spectrometry. Anal Biochem, 417 (1), 25-35. doi: 10.1016/j.ab.2011.05.005
119 de Felipe P (2002) Polycistronic viral vectors. Curr Gene Ther 2:355-378. doi: 10.2174/1566523023347742.
120 Foote, J., Winter, G. (1992). Antibody framework residues affecting the conformation of the hypervariable loops. J Mol Biol, 224 (2), 487-499.
121 Massie, B., Dionne, J., Lamarche, N., Fleurent, J., Langelier, Y. (1995). Improved adenovirus vector provides herpes simplex virus ribonucleotide reductase R1 and R2 subunits very efficiently. Nature Biotechnology, 13 (6), 602-608.
122 Kim, D. W., Uetsuki, T., Kaziro, Y., Yamaguchi, N., 8 Sugano, S. (1990). Use of the human elongation factor 1 alpha promoter as a versatile and efficient expression system. Gene, 91 (2), 217-223.
123 Holden, P., Keene, D. R., Lunstrum, G. P., Bachinger, H. P., 8 Horton, W. A. (2005). Secretion of cartilage oligomeric matrix protein is affected by the signal peptide. J Biol Chem, 280 (17), 17172-17179.
124 Sadelain, M., Papapetrou, E. P., 8 Bushman, F. D. (2012). Safe harbours for the integration of new DNA in the human genome. Nature Reviews Cancer, 12 (1), 51-58.
125 Sanjana, N. E., Cong, L., Zhou, Y., Cunniff, M. M., Feng, G., 8 Zhang, F. (2012). A transcription activator-like effector toolbox for genome engineering. Nat Protoc, 7 (1), 171-192. doi: 10.1038/n prot. 2011.431
126 Falk, R., Falk, A., Dyson, M. R., Melidoni, A. N., Parthiban, K., Young, J. L., et al. (2012). Generation of anti-Notch antibodies and their application in blocking Notch signalling in neural stem cells. Methods, 58 (1), 69-78. doi: 10.1016/j.ymeth. 2012.07.008
127 Martin, C. D., Rojas, G., Mitchell, J. N., Vincent, K. J., Wu, J., McCafferty, J., Schofield, D. J. (2006). A simple vector system to improve performance and utilisation of recombinant antibodies. BMC Biotechnology, 6, 46.
128 Reyon, D., Tsai, S. Q., Khgayter, C., Foden, J. A., Sander, J. D., Joung, J. K. (2012). FLASH assembly of TALENs for high-throughput genome editing. Nature Biotechnology, 30 (5), 460-465. doi: 10.1038/nbt. 2170
129 Van Der Weyden, L., Adams, D. J., Harris, L. W., Tannahill, D., Arends, M. J., Bradley, A. (2005). Null and conditional Semaphorin 3B alleles using a flexible purohtk LoxP/FRT vector. Genesis, 44 (4), 171-178
130 de Felipe, P., 8 Ryan, M. D. (2004). Targeting of proteins derived from self-processing polyproteins containing multiple signal sequences. Traffic, 5 (8), 616-626.
131 Raymond, C. S., & Soriano, P. (2007). High-efficiency FLP and PhiC31 site-specific recombination in mammalian cells. PLOS ONE, 2 (1), e162. doi: 10.1371/journal. pone. 0000162
132 Kranz, A., Fu, J., Duerschke, K., Weidlich, S., Naumann, R., Stewart, A. F., Anastassiadis, K. (2010). An improved Flp deleter mouse in C57Bl/6 based on Flpo recombinase. Genesis, 48 (8), 512-520. doi: 10.1002/dvg. 20641
133 Szymczak A L, Vignali D A (2005) Development of 2A peptide-based strategies in the design of multicistronic vectors. Expert Opin Biol Ther 5:627-638. doi: 10.1517/14712598.5.5.627
134 Chapple, S. D., Crafts, A. M., Shadbolt, S. P., McCafferty, J., and Dyson, M. R. (2006). Multiplexed expression and screening for recombinant protein production in mammalian cells. BMC biotechnology 6, 49.
135. Zhao, Y. et al. Multiple injections of electroporated autologous T cells expressing a chimeric antigen receptor mediate regression of human disseminated tumor. Cancer Research 70, 9053-9061 (2010).
136 Szymczak, A. L. et al. Correction of multi-gene deficiency in vivo using a single ‘selfcleaving’ 2A peptide-based retroviral vector. Nature biotechnology 22, 589-594 (2004).
137. Li, Y. et al. Directed evolution of human T-cell receptors with picomolar affinities by phage display. Nat. Biotechnol. 23, 349-354 (2005).
138. Zhao, Y. et al. High-affinity TCRs generated by phage display provide CD4+ T cells with the ability to recognize and kill tumor cell lines. J. Immunol. 179, 5845-5854 (2007).
139. Madura, F. et al. T-cell receptor specificity maintained by altered thermodynamics. The Journal of biological chemistry 288, 18766-18775 (2013).
140. Pierce, B. G. et al. Computational design of the affinity and specificity of a therapeutic T cell receptor. PLOS Comput Biol 10, e1003478 (2014).
141. Sebestyen, Z. et al. Human TCR that incorporate CD3zeta induce highly preferred pairing between TCRalpha and beta chains following gene transfer. J. Immunol. 180, 7736-7746 (2008).
142. Roszik, J. et al. T-cell synapse formation depends on antigen recognition but not CD3 interaction: studies with TCR: (a candidate transgene for TCR gene therapy. Eur. J. Immunol. 41, 1288-1297 (2011).
143. Cohen, C. J., Zhao, Y., Zheng, Z., Rosenberg, S. A. Morgan, R. A. Enhanced antitumor activity of murine-human hybrid T-cell receptor (TCR) in human lymphocytes is associated with improved pairing and TCRICD3 stability. Cancer Research 66, 8878-8886 (2006).
144. Huovinen, T. et al. Primer extension mutagenesis powered by selective rolling circle amplification. PLOS ONE 7, e31817 (2012).
145. Cribbs, A. P., Kennedy, A., Gregory, B. & Brennan, F. M. Simplified production and concentration of lentiviral vectors to achieve high transduction in primary human T cells. BMC biotechnology 13, 98 (2013).
146. Oelke, M. et al. Ex vivo induction and expansion of antigen-specific cytotoxic T cells by HLA-Ig-coated artificial antigen-presenting cells. Nat Med 9, 619-624 (2003).
147. Wolf I, M. Greenberg, P. D. Antigen-specific activation and cytokine-facilitated expansion of naive, human CD8+ T cells. Nature Protocols 9, 950-966 (2014).
148. Lipowska-Bhalla, G., Gilham, D. E., Hawkins, R. E. & Rothwell, D. G. Isolation of tumor antigen-specific single-chain variable fragments using a chimeric antigen receptor bicistronic retroviral vector in a Mammalian screening protocol. Hum Gene Ther Methods 24, 381-391 (2013).
149. Kelly, R. J., Sharon, E., Pastan, I. Hassan, R. Mesothelin-targeted agents in clinical trials and in preclinical development. Mol. Cancer Ther. 11, 517-525 (2012).
150. Atanackovic, D. et al. Surface molecule CD229 as a novel target for the diagnosis and treatment of multiple myeloma. Haematologica 96, 1512-1520 (2011).
151. Bund, D., Mayr, C., Kofler, D. M., Hallek, M. & Wendtner, C.-M. Human Ly9 (CD229) as novel tumor-associated antigen (TAA) in chronic lymphocytic leukemia (B-CLL) recognized by autologous CD8+ T cells. Exp. Hematol. 34, 860-869 (2006).
152. Tiede, C. et al. Adhiron: a stable and versatile peptide display scaffold for molecular recognition applications. Protein Eng. Des. Sel. 27, 145-155 (2014).
153. Maresca, M., Lin, V. G., Guo, N. & Yang, Y. Obligate ligation-gated recombination (ObLiGaRe): custom-designed nuclease-mediated targeted integration through nonhomologous end joining. Genome Res. 23, 539-546 (2013).
154. McVey, M. Lee, S. E. MMEJ repair of double-strand breaks (director's cut): deleted sequences and alternative endings. Trends Genet. 24, 529-538 (2008).
155. Nakade, S. et al. Microhomology-mediated end-joining-dependent integration of donor DNA in cells and animals using TALENs and CRISPRICas9. Nat Commun 5, 5560 (2014).
156. Chiche, L. et al. Squash inhibitors: From structural motifs to macrocyclic knottins. Current Protein 8 Peptide Science 5, 341-349.

1. A method for identifying a locus in a genome of a eukaryotic cell, said locus being a candidate for insertion of binder sequences, said method comprising:
a. providing a landing pad sequence;
b. introducing the landing pad sequence into the eukaryotic cell;
c. randomly integrating the landing pad sequence into the genome of the eukaryotic cell via transposon-mediated integration;
d. selecting a clone having a landing pad sequence integrated into its genome.

2. The method of claim 1 comprising the further steps of:
e. screening for single-copy integration;
f. identifying the locus.

3. The method of claim 2 comprising the additional steps of:
g. integrating a donor DNA sequence comprising one or more transgenes encoding a binder at the landing pad sequence;
h. screening for integration of the donor DNA.

4. The method of any one of claims 1-3, wherein the landing pad sequence comprises a recognition sequence for a site-specific nuclease, preferably wherein the nuclease recognition sequence is a meganuclease recognition sequence, a zinc finger nuclease recognition sequence, a TALE nuclease recognition sequence or a nucleic acid guided nuclease recognition sequence, preferably a meganuclease recognition sequence, preferably a I-SceI meganuclease recognition sequence.

5. The method of claim 4, wherein step g of integrating the donor DNA into the cells comprises providing a site-specific nuclease within the cells, wherein the nuclease cleaves the recognition sequence comprised in the landing pad.

6. The method of any one of claims 3-5, wherein step h of screening for integration of the donor DNA comprises screening for display of the one or more binders encoded by the donor DNA.

7. The method of any one of claims 3-6, wherein the donor DNA further comprises homology arms to increase integration efficiency.

8. The method of any one of claims 1-7, wherein the landing pad sequence and/or the donor DNA sequence comprise a selectable marker.

9. Use of the locus identified in the method of any one of claims 1 through 8 for building a library of eukaryotic cell clones containing DNA encoding a diverse repertoire of binders.

10. An in vitro library of eukaryotic cell clones that express a diverse repertoire of at least 10\hat{ }3, 10\hat{ }4, 10\hat{ }5, 10\hat{ }6, 10\hat{ }7, 10\hat{ }8 or 10\hat{ }9 different binders, each cell containing recombinant DNA wherein donor DNA encoding a binder or subunit of a binder is integrated in at least a first and/or a second fixed locus in the cellular DNA, said locus or loci being identified by a method according to any one of claims 1-8, preferably wherein said locus or loci are in a gene selected from an NLN gene, a TNIK gene, a PARP11 gene, a RAB40B gene, an ABI2 gene, an RNF19B gene, a PKIA gene, or an FTCD gene, more preferably wherein the locus or loci are in an NLN gene, a TNIK gene or a RAB40B gene, most preferably in an NLN gene.

11. An in vitro library of eukaryotic cell clones according to claim 10, wherein the locus or loci are in an intron of the gene, preferably wherein the locus or loci are in an open chromatin region of the intron and/or wherein the locus or loci are in an enhancer region of the intron.

12. An in vitro library of eukaryotic cell clones according to claim 10 or 11, wherein the locus or loci are in NLN-207 intron 1, 2 or 6 of the NLN gene.

13. A binder identified from a library according to any of claims 10-12.

14. A method for producing a library of eukaryotic cell clones containing DNA encoding a diverse repertoire of binders, comprising:
providing donor DNA molecules encoding the binders, and eukaryotic cells;
introducing the donor DNA into the cells and providing a site-specific nuclease within the cells, wherein the nuclease cleaves a recognition sequence in cellular DNA, wherein the recognition sequence is in an NLN gene, a TNIK gene, a PARP11 gene, a RAB40B gene, an ABI2 gene, an RNF19B gene, a PKIA gene, or an FTCD gene, preferably in an NLN gene, a TNIK gene or a RAB40B gene, more preferably in an NLN gene, to create an integration site at which the donor DNA becomes integrated into the cellular DNA, integration occurring through DNA repair mechanisms endogenous to the cells, thereby creating recombinant cells containing donor DNA integrated in the cellular DNA; and
culturing the recombinant cells to produce clones, thereby providing a library of eukaryotic cell clones containing donor DNA encoding the repertoire of binders.

15. A method according to claim 14, wherein the recognition sequence is in an intron of the gene, preferably wherein the recognition sequence is in an open chromatin region of the intron and/or wherein the recognition sequence is in an enhancer region of the intron.

16. A method according to claim 14 or 15, wherein the recognition sequence is in NLN-207 intron 1, 2 or 6 of the NLN gene.
